(19) United States
(12) Patent Application Publication
(10) Pub. No.: US 2016/0120693 A1
(43) Pub. Date: May 5, 2016

(54) Eye Care Devices and Methods

(71) Applicant: OPTOMETRIC TECHNOLOGY GROUP LTD., London (GB)

(72) Inventors: Michel Lucien GUILLON, St. Laurence (JE); Cecile Adrienne MAISSA, London (GB)

(73) Assignee: Optometric Technology Group, London (GB)

(21) Appl. No.: 14/889,677

(22) PCT Filed: May 8, 2014
(86) PCT No.: PCT/IB2014/001705

§ 371 Date: Nov. 6, 2015

Related U.S. Application Data
(60) Provisional application No. 61/821,208, filed on May 8, 2013.

(51) Int. Cl. A61F 7/03; A61F 9/00

(52) U.S. Cl. CPC A61F 7/03; A61F 9/0008; A61F 2007/0004

(57) ABSTRACT

Wipes and their use in methods of treatment of eye disorders such as dry eye or meibomian gland dysfunction.

### CROSS-REFERENCE

This application claims the benefit of U.S. Provisional Application No. 61/821,208, filed on May 8, 2013, which is incorporated herein by reference in its entirety.

### BACKGROUND

Meibomian glands, which are positioned throughout the eyelid margins, provide lipid like secretions (known as meibum) to the surface of the eye. When blinking occurs, the upper eyelid moves downwardly over the eye and compresses the lipids between the margins of the eyelids. Upon eye opening, the upper lid moves upwardly and pulls a sheet of lipid upwardly to form a film over the eye. This lipid sheet coats the aqueous part of the tear layer which in turn coats the surface of the eye. The presence of this lipid sheet restricts evaporation of the tear layer such that the surface of the eye is maintained in a moist environment. Failure of the meibomian glands can mean that the required lipid layer is not properly formed and evaporation of the tear layer can occur rapidly which can lead to sensations of dryness, irritation and burning.

### BRIEF SUMMARY

In one aspect, the present disclosure relates to wipes, for example eyelid wipes.

In a further aspect, the present disclosure relates to a method of treatment of disorders of the eyelid margin such as dry eye or those caused by meibomian gland dysfunction.

In one of many aspects, provided herein is a method of treating an eyelid disorder in a subject, comprising applying a wipe to an eye region of the subject, wherein the wipe generates heat and is adapted for massage, and wherein the application of the wipe increases stability of a tear film of the subject, modulates a lipid concentration in a tear film of the subject, and/or modulates a lipid proportion in a total lipid composition of a tear film of the subject.

In one aspect, provided herein is a method of increasing stability of a tear film of a subject, modulating a lipid concentration in a tear film of a subject, and/or modulating a lipid proportion in a total lipid composition of the tear film of a subject, comprising applying a wipe to an eye region of the subject, wherein the wipe generates heat and is adapted for massage, and wherein the subject suffers from an eyelid disorder.

In some instances, the eyelid disorder is blepharitis, dryness, irritation, redness, inflammation, meibomian gland dysfunction, hay fever, a condition associated with use of eye cosmetics, or a condition associated with contact-lens wear. In one instance, the eyelid disorder is meibomian gland dysfunction.

In one aspect, a non-invasive break-up time (NIBUT) of a tear film of the subject is increased after the application of the wipe in comparison to a NIBUT of a tear film of the subject before the application of the wipe.

In some instances, the NIBUT of the tear film of the subject is increased after the application of the wipe by about 30%-100% in comparison to the NIBUT of the tear film of the subject before the application of the wipe. In some instances, the NIBUT of the tear film of the subject is increased after the application of the wipe by about: 30%-40%, 40%-50%, 50%-60%, 60%-70%, 70%-80%, 80%-90%, or 90%-100% in comparison to the NIBUT of the tear film of the subject before the application of the wipe. In some instances, the NIBUT of the tear film of the subject is increased after the application of the wipe by about: 30%-90%, 40%-80%, 50%-70%, or 55%-65% in comparison to the NIBUT of the tear film of the subject before the application of the wipe. In some instances, the NIBUT of the tear film of the subject is increased after the application of the wipe by about: 30%, 35%, 40%, 45%, 50%, 55%, 60%, 65%, 70%, 75%, 80%, 85%, 90%, 95%, or 100% in comparison to the NIBUT of the tear film of the subject before the application of the wipe. In some instances, the NIBUT of the tear film of the subject is increased by about 55%-65% in about 1-14 days after beginning the application of the wipe in comparison to the NIBUT of the tear film of the subject before the application of the wipe. In one instance, the NIBUT of the tear film of the subject is increased by about 58% in about 7 days after beginning the application of the wipe in comparison to the NIBUT of the tear film of the subject before the application of the wipe. In some instances, the NIBUT of the tear film of the subject is increased by about 60%-70% in about 1-42 days after beginning the application of the wipe in comparison to the NIBUT of the tear film of the subject before the application of the wipe. In one instance, the NIBUT of the tear film of the subject is increased by about 65% in about 21 days after beginning the application of the wipe in comparison to the NIBUT of the tear film of the subject before the application of the wipe.

In some instances, the NIBUT of the tear film of the subject has an increase of about 1-10 seconds after the application of the wipe in comparison to the NIBUT of the tear film of the subject before the application of the wipe. In some instances, the NIBUT of the tear film of the subject has an increase of about: 1-2, 2-3, 3-4, 4-5, 5-6, 6-7, 7-8, 8-9, or 9-10 seconds after the application of the wipe in comparison to the NIBUT of the tear film of the subject before the application of the wipe. In some instances, the NIBUT of the tear film of the subject has an increase of about: 1-9, 2-8, 3-7, 4-6, or 4.5-5.5 seconds after the application of the wipe in comparison to the NIBUT of the tear film of the subject before the application of the wipe. In some instances, the NIBUT of the tear film of the subject has an increase of about: 1, 1.5, 2, 2.5, 3, 3.5, 4, 4.5, 5, 5.5, 6, 6.5, 7, 7.5, 8, 8.5, 9, 9.5, or 10 seconds after the application of the wipe in comparison to the NIBUT of the tear film of the subject before the application of the wipe. In some instances, the NIBUT of the tear film of the subject has an increase of about 4-5 seconds in about 1-14 days after beginning the application of the wipe in comparison to the NIBUT of the tear film of the subject before the application of the wipe. In one instance, the NIBUT of the tear film of the subject has an increase of about 4.5 seconds in about 7 days after beginning the application of the wipe in comparison to the NIBUT of the tear film of the subject before the application of the wipe. In some instances, the NIBUT of the tear film of the subject has an increase of about 4-6 seconds in about 1-42 days after beginning the application of the wipe in comparison to the NIBUT of the tear film of the subject before the application of the wipe. In one instance, the NIBUT of the tear film of the subject has an increase of about 5 seconds in about 21 days after beginning the application of the wipe in comparison to the NIBUT of the tear film of the subject before the application of the wipe.

In one aspect, a total lipid concentration in a tear film of the subject is increased after the application of the wipe in comparison to a total lipid concentration of a tear film of the subject before the application of the wipe.

In some instances, the total lipid concentration in the tear film of the subject is increased after the application of the wipe by about 50%-200% in comparison to the total lipid concentration of the tear film of the subject before the application of the wipe. In some instances, the total lipid concentration in the tear film of the subject is increased after the application of the wipe by about: 50%-60%, 60%-70%, 70%-80%, 80%-90%, 90%-100%, 100%-110%, 110%-120%, 120%-130%, 130%-140%, 140%-150%, 150%-160%, 160-170%, 170%-180%, 180%-190%, or 190%-200% in comparison to the total lipid concentration of the tear film of the subject before the application of the wipe. In some instances, the total lipid concentration in the tear film of the subject is increased after the application of the wipe by about: 50%-100%, 60%-110%, 70%-90%, 100%-160%, 110%-150%, or 120%-140% in comparison to the total lipid concentration of the tear film of the subject before the application of the wipe. In some instances, the total lipid concentration in the tear film of the subject is increased after the application of the wipe by about: 50%, 60%, 70%, 80%, 90%, 100%, 110%, 120%, 130%, 140%, 150%, 160%, 170%, 180%, 190%, or 200% in comparison to the total lipid concentration of the tear film of the subject before the application of the wipe. In some instances, the total lipid concentration in the tear film of the subject is increased by about 75-85% in about 1-42 days after beginning the application of the wipe in comparison to the total lipid concentration of the tear film of the subject before the application of the wipe. In one instance, the total lipid concentration in the tear film of the subject is increased by about 81% in about 21 days after beginning the application of the wipe in comparison to the total lipid concentration of the tear film of the subject before the application of the wipe. In some instances, the total lipid concentration in the tear film of the subject is increased by about 120-140% in about 60-120 days after beginning the application of the wipe in comparison to the total lipid concentration of the tear film of the subject before the application of the wipe. In one instance, the total lipid concentration in the tear film of the subject is increased by about 131% in about 90 days after beginning the application of the wipe in comparison to the total lipid concentration of the tear film of the subject before the application of the wipe.

In some instances, the total lipid concentration in the tear film of the subject has an increase of about 1-10 μg/μl after the application of the wipe in comparison to the total lipid concentration of the tear film of the subject before the application of the wipe. In some instances, the total lipid concentration in the tear film of the subject has an increase of about: 1-2 μg/μl, 2-3 μg/μl, 3-4 μg/μl, 4-5 μg/μl, 5-6 μg/μl, 6-7 μg/μl, 7-8 μg/μl, 8-9 μg/μl, or 9-10 μg/μl after the application of the wipe in comparison to the total lipid concentration of the tear film of the subject before the application of the wipe. In some instances, the total lipid concentration in the tear film of the subject has an increase of about: 1-6 μg/μl, 2-5 μg/μl, 2-3.5 μg/μl, or 3.5-5 μg/μl after the application of the wipe in comparison to the total lipid concentration of the tear film of the subject before the application of the wipe. In some instances, the total lipid concentration in the tear film of the subject has an increase of about: 1, 1.5, 2, 2.5, 3, 3.5, 4, 4.5, 5, 5.5, 6, 6.5, 7, 7.5, 8, 8.5, 9, 9.5, or 10 μg/μl after the application of the wipe in comparison to the total lipid concentration of the tear film of the subject before the application of the wipe. In some instances, the total lipid concentration in the tear film of the subject has an increase of about 2-3 μg/μl in about 1-42 days after beginning the application of the wipe in comparison to the total lipid concentration of the tear film of the subject before the application of the wipe. In one instance, the total lipid concentration in the tear film of the subject has an increase of about 2.6 μg/μl in about 21 days after beginning the application of the wipe in comparison to the total lipid concentration of the tear film of the subject before the application of the wipe. In some instances, the total lipid concentration in the tear film of the subject has an increase of about 4-5 μg/μl in about 60-120 days after beginning the application of the wipe in comparison to the total lipid concentration of the tear film of the subject before the application of the wipe. In one instance, the total lipid concentration in the tear film of the subject has an increase of about 4.2 μg/μl in about 90 days after beginning the application of the wipe in comparison to the total lipid concentration of the tear film of the subject before the application of the wipe.

In one aspect, a proportion of wax ester or cholesterol ester in a total lipid composition of a tear film of the subject is increased after the application of the wipe in comparison to a proportion of wax ester or cholesterol ester in a total lipid composition of a tear film of the subject before the application of the wipe.

In some instances, the proportion of wax ester or cholesterol ester in the total lipid composition of the tear film of the subject is increased after the application of the wipe by about 1%-20% in comparison to the proportion of wax ester or cholesterol ester in the total lipid composition of the tear film of the subject before the application of the wipe. In some instances, the proportion of wax ester or cholesterol ester in the total lipid composition of the tear film of the subject is increased after the application of the wipe by about: 1%-2%, 2%-4%, 4%-6%, 6%-8%, 8%-10%, 10%-12%, 12%-14%, 14%-16%, 16%-18%, or 18%-20% in comparison to the proportion of wax ester or cholesterol ester in the total lipid composition of the tear film of the subject before the application of the wipe. In some instances, the proportion of wax ester or cholesterol ester in the total lipid composition of the tear film of the subject is increased after the application of the wipe by about: 1%-8%, 2%-7%, 3%-6%, 2%-4%, 5%-7%, 4%-8%, or 1%-5% in comparison to the proportion of wax ester or cholesterol ester in the total lipid composition of the tear film of the subject before the application of the wipe. In some instances, the proportion of wax ester or cholesterol ester in the total lipid composition of the tear film of the subject is increased after the application of the wipe by about: 1%, 2%, 3%, 4%, 5%, 6%, 7%, 8%, 9%, 10%, 11%, 12%, 13%, 14%, 15%, 16%, 17%, 18%, 19%, or 20% in comparison to the proportion of wax ester or cholesterol ester in the total lipid composition of the tear film of the subject before the application of the wipe. In some instances, the proportion of wax ester or cholesterol ester in the total lipid composition of the tear film of the subject is increased by about 2-4% in about 1-42 days after beginning the application of the wipe in comparison to the proportion of wax ester or cholesterol ester in the total lipid composition of the tear film of the subject before the application of the wipe. In one instance, the proportion of wax ester or cholesterol ester in the total lipid composition of the tear film of the subject is increased by about 3% in about 21 days after beginning the application of the wipe in comparison to the proportion of wax ester or cholesterol ester in the total lipid composition of the tear film of the subject before the application of the wipe. In some instances, the proportion of wax ester or cholesterol ester in the total lipid composition of the tear film of the subject is increased by about 5-7% in about 60-120 days after beginning the application of the wipe in comparison to the proportion of wax ester or cholesterol ester in the total lipid composition of the tear film of the subject before the application of the wipe. In one instance, the proportion of wax ester or cholesterol ester in the total lipid composition of the tear film of the subject is increased by about 6% in about 90 days after beginning the application of the wipe in comparison to the proportion of wax ester or cholesterol ester in the total lipid composition of the tear film of the subject before the application of the wipe.

In one aspect, a proportion of wax ester and cholesterol ester in a total lipid composition of a tear film of the subject has an increase of about 1%-10% after the application of the wipe in comparison to a proportion of wax ester and cholesterol ester in a total lipid composition of a tear film of the subject before the application of the wipe. In some instances, the proportion of wax ester and cholesterol ester in the total lipid composition of the tear film of the subject has an increase of about: 1%-2%, 2%-3%, 3%-4%, 4%-5%, 5%-6%, 6%-7%, 7%-8%, 8%-9%, or 9%-10% after the application of the wipe in comparison to the proportion of wax ester and cholesterol ester in the total lipid composition of the tear film of the subject before the application of the wipe. In some instances, the proportion of wax ester and cholesterol ester in the total lipid composition of the tear film of the subject has an increase of about: 1%-7%, 2-6%, 1.5%-3.5%, or 4.5%-6.5% after the application of the wipe in comparison to the proportion of wax ester and cholesterol ester in the total lipid composition of the tear film of the subject before the application of the wipe. In some instances, the proportion of wax ester and cholesterol ester in the total lipid composition of the tear film of the subject has an increase of about: 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10% after the application of the wipe in comparison to the proportion of wax ester and cholesterol ester in the total lipid composition of the tear film of the subject before the application of the wipe. In some instances, the proportion of wax ester and cholesterol ester in the total lipid composition of the tear film of the subject has an increase of about 2-3% in about 1-42 days after beginning the application of the wipe in comparison to the proportion of wax ester and cholesterol ester in the total lipid composition of the tear film of the subject before the application of the wipe. In one instance, the proportion of wax ester and cholesterol ester in the total lipid composition of the tear film of the subject has an increase of about 2.4% in about 21 days after beginning the application of the wipe in comparison to the proportion of wax ester and cholesterol ester in the total lipid composition of the tear film of the subject before the application of the wipe. In some instances, the proportion of wax ester and cholesterol ester in the total lipid composition of the tear film of the subject has an increase of about 4-6% in about 60-120 days after beginning the application of the wipe in comparison to the proportion of wax ester and cholesterol ester in the total lipid composition of the tear film of the subject before the application of the wipe. In one instance, the proportion of wax ester and cholesterol ester in the total lipid composition of the tear film of the subject has an increase of about 5.2% in about 90 days after beginning the application of the wipe in comparison to the proportion of wax ester and cholesterol ester in the total lipid composition of the tear film of the subject before the application of the wipe.

In one aspect, a proportion of fatty acids in a total lipid composition of a tear film of the subject is decreased after the application of the wipe in comparison to a proportion of fatty acids in the total lipid composition of a tear film of the subject before the application of the wipe.

In some instances, the proportion of fatty acids in the total lipid composition of the tear film of the subject is decreased after the application of the wipe by about 5%-80% in comparison to the proportion of fatty acids in the total lipid composition of the tear film of the subject before the application of the wipe. In some instances, the proportion of fatty acids in the total lipid composition of the tear film of the subject is decreased after the application of the wipe by about 5%-10%, 10%-15%, 15%-20%, 20%-25%, 25%-30%, 30%-35%, 35%-40%, 40%-45%, 45%-50%, 50%-55%, 55%-60%, 65%-70%, 70%-75%, or 75%-80% in comparison to the proportion of fatty acids in the total lipid composition of the tear film of the subject before the application of the wipe. In some instances, the proportion of fatty acids in the total lipid composition of the tear film of the subject is decreased after the application of the wipe by about 5%-60%, 10%-50%, 5%-25%, 5%-30%, 10%-20%, 40%-45%, 30%-50%, 30%-60%, 20%-60%, or 10%-70% in comparison to the proportion of fatty acids in the total lipid composition of the tear film of the subject before the application of the wipe. In some instances, the proportion of fatty acids in the total lipid composition of the tear film of the subject is decreased after the application of the wipe by about: 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, or 80% in comparison to the proportion of fatty acids in the total lipid composition of the tear film of the subject before the application of the wipe. In some instances, the proportion of fatty acids in the total lipid composition of the tear film of the subject is decreased by about 5-30% in about 1-42 days after beginning the application of the wipe in comparison to the proportion of fatty acids in the total lipid composition of the tear film of the subject before the application of the wipe. In one instance, the proportion of fatty acids in the total lipid composition of the tear film of the subject is decreased by about 16% in about 21 days after beginning the application of the wipe in comparison to the proportion of fatty acids in the total lipid composition of the tear film of the subject before the application of the wipe. In some instances, the proportion of fatty acids in the total lipid composition of the tear film of the subject is decreased by about 30-60% in about 60-120 days after beginning the application of the wipe in comparison to the proportion of fatty acids in the total lipid composition of the tear film of the subject before the application of the wipe. In one instance, the proportion of fatty acids in the total lipid composition of the tear film of the subject is decreased by about 43% in about 90 days after beginning the application of the wipe in comparison to the proportion of fatty acids in the total lipid composition of the tear film of the subject before the application of the wipe.

In some instances, the proportion of fatty acids in the total lipid composition of the tear film of the subject has a decrease of about 1%-10% after the application of the wipe in comparison to the proportion of fatty acids in the total lipid composition of the tear film of the subject before the application of the wipe. In some instances, the proportion of fatty acids in the total lipid composition of the tear film of the subject has a decrease of about: 1%-2%, 2%-3%, 3%-4%, 4%-5%, 5%-6%, 6%-7%, 7%-8%, 8%-9%, or 9%-10% after the application of the wipe in comparison to the proportion of fatty acids in the total lipid composition of the tear film of the subject before the application of the wipe. In some instances, the proportion of fatty acids in the total lipid composition of the tear film of the subject has a decrease of about: 1%-7%, 1%-6%, 1%-3%, 4%-6%, or 4%-7% after the application of the wipe in comparison to the proportion of fatty acids in the total lipid composition of the tear film of the subject before the application of the wipe. In some instances, the proportion of fatty acids in the total lipid composition of the tear film of the subject has a decrease of about: 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10% after the application of the wipe in comparison to the proportion of fatty acids in the total lipid composition of the tear film of the subject before the application of the wipe. In some instances, the proportion of fatty acids in the total lipid composition of the tear film of the subject has a decrease of about 1-3% in about 1-42 days after beginning the application of the wipe in comparison to the proportion of fatty acids in the total lipid composition of the tear film of the subject before the application of the wipe. In one instance, the proportion of fatty acids in the total lipid composition of the tear film of the subject has a decrease of about 2% in about 21 days after beginning the application of the wipe in comparison to the proportion of fatty acids in the total lipid composition of the tear film of the subject before the application of the wipe. In some instances, the proportion of fatty acids in the total lipid composition of the tear film of the subject has a decrease of about 4-7% in about 60-120 days after beginning the application of the wipe in comparison to the proportion of fatty acids in the total lipid composition of the tear film of the subject before the application of the wipe. In one instance, the proportion of fatty acids in the total lipid composition of the tear film of the subject has a decrease of about 5.4% in about 90 days after beginning the application of the wipe in comparison to the proportion of fatty acids in the total lipid composition of the tear film of the subject before the application of the wipe.

In one aspect, a proportion of triglycerides in a total lipid composition of a tear film of the subject is increased after the application of the wipe in comparison to a proportion of triglycerides in a total lipid composition of a tear film of the subject before the application of the wipe.

In some instances, the proportion of triglycerides in the total lipid composition of the tear film of the subject is increased after the application of the wipe by about 30%-200% in comparison to the proportion of triglycerides in the total lipid composition of the tear film of the subject before the application of the wipe. In some instances, the proportion of triglycerides in the total lipid composition of the tear film of the subject is increased after the application of the wipe by about: 30%-40%, 40%-50%, 50%-60%, 60%-70%, 70%-80%, 80%-90%, 90%-100%, 100%-110%, 110%-120%, 120%-130%, 130%-140%, 140%-150%, 150%-160%, 160-170%, 170%-180%, 180%-190%, or 190%-200% in comparison to the proportion of triglycerides in the total lipid composition of the tear film of the subject before the application of the wipe. In some instances, the proportion of triglycerides in the total lipid composition of the tear film of the subject is increased after the application of the wipe by about: 30%-70%, 40%-60%, 130%-190%, 140%-180%, 30%-180%, 40%-170%, or 50%-160% in comparison to the proportion of triglycerides in the total lipid composition of the tear film of the subject before the application of the wipe. In some instances, the proportion of triglycerides in the total lipid composition of the tear film of the subject is increased after the application of the wipe by about: 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, or 200% in comparison to the proportion of triglycerides in the total lipid composition of the tear film of the subject before the application of the wipe. In some instances, the proportion of triglycerides in the total lipid composition of the tear film of the subject is increased by about 40-60% in about 1-42 days after beginning the application of the wipe in comparison to the proportion of triglycerides in the total lipid composition of the tear film of the subject before the application of the wipe. In one instance, the proportion of triglycerides in the total lipid composition of the tear film of the subject is increased by about 51% in about 21 days after beginning the application of the wipe in comparison to the proportion of triglycerides in the total lipid composition of the tear film of the subject before the application of the wipe. In some instances, the proportion of triglycerides in the total lipid composition of the tear film of the subject is increased by about 120%-180% in about 60-120 days after beginning the application of the wipe in comparison to the proportion of triglycerides in the total lipid composition of the tear film of the subject before the application of the wipe. In one instance, the proportion of triglycerides in the total lipid composition of the tear film of the subject is increased by about 151% in about 90 days after beginning the application of the wipe in comparison to the proportion of triglycerides in the total lipid composition of the tear film of the subject before the application of the wipe.

In some instances, the proportion of triglycerides in the total lipid composition of the tear film of the subject has an increase of about 0.1%-3% after the application of the wipe in comparison to the proportion of triglycerides in the total lipid composition of the tear film of the subject before the application of the wipe. In some instances, the proportion of triglycerides in the total lipid composition of the tear film of the subject has an increase of about: 0.1%-1%, 0.2%-0.9%, 0.2%-0.4%, 0.25%-0.35%, 0.75%-0.85%, 0.7%-0.9%, 0.6%-1.0%, or 0.1%-1.5% after beginning the application of the wipe in comparison to the proportion of triglycerides in the total lipid composition of the tear film of the subject before the application of the wipe. In some instances, the proportion of triglycerides in the total lipid composition of the tear film of the subject has an increase of about: 0.1%-0.2%, 0.2%-0.4%, 0.4%-0.6%, 0.6%-0.8%, 0.8%-1.0%, 1%-1.2%, 1.2%-1.4%, 1.4%-1.6%, 1.6%-1.8%, 1.8%-2%, 2%-2.2%, 2.2%-2.4%, 2.4%-2.6%, 2.6%-2.8%, or 2.8%-3% after beginning the application of the wipe in comparison to the proportion of triglycerides in the total lipid composition of the tear film of the subject before the application of the wipe. In some instances, the proportion of triglycerides in the total lipid composition of the tear film of the subject has an increase of about: 0.1%, 0.15%, 0.2%, 0.25%, 0.3%, 0.35%, 0.4%, 0.45%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.5%, or 3% after the application of the wipe in comparison to the proportion of triglycerides in the total lipid composition of the tear film of the subject before the application of the wipe. In some instances, the proportion of triglycerides in the total lipid composition of the tear film of the subject has an increase of about 0.2-0.4% in about 1-42 days after beginning the application of the wipe in comparison to the proportion of triglycerides in the total lipid composition of the tear film of the subject before the application of the wipe. In one instance, the proportion of triglycerides in the total lipid composition of the tear film of the subject has an increase of about 0.3% in about 21 days after beginning the application of the wipe in comparison to the proportion of triglycerides in the total lipid composition of the tear film of the subject before the application of the wipe. In some instances, the proportion of triglycerides in the total lipid composition of the tear film of the subject has an increase of about 0.6-1.0% in about 60-120 days after beginning the application of the wipe in comparison to the proportion of triglycerides in the total lipid composition of the tear film of the subject before the application of the wipe. In one instance, the proportion of triglycerides in the total lipid composition of the tear film of the subject has an increase of about 0.8% in about 90 days after beginning the application of the wipe in comparison to the proportion of triglycerides in the total lipid composition of the tear film of the subject before the application of the wipe.

In one aspect, a proportion of cholesterol in a total lipid composition of a tear film of the subject is decreased after the application of the wipe in comparison to a proportion of cholesterol in a total lipid composition of a tear film of the subject before the application of the wipe.

In some instances, the proportion of cholesterol in the total lipid composition of the tear film of the subject is decreased after the application of the wipe by about 10%-80% in comparison to the proportion of cholesterol in the total lipid composition of the tear film of the subject before the application of the wipe. In some instances, the proportion of cholesterol in the total lipid composition of the tear film of the subject is decreased after the application of the wipe by about: 10%-15%, 15%-20%, 20%-25%, 25%-30%, 30%-35%, 35%-40%, 40%-45%, 45%-50%, 50%-55%, 55%-60%, 65%-70%, 70%-75%, or 75%-80% in comparison to the proportion of cholesterol in the total lipid composition of the tear film of the subject before the application of the wipe. In some instances, the proportion of cholesterol in the total lipid composition of the tear film of the subject is decreased after the application of the wipe by about: 10%-70%, 20%-60%, or 30%-50% in comparison to the proportion of cholesterol in the total lipid composition of the tear film of the subject before the application of the wipe. In some instances, proportion of cholesterol in the total lipid composition of the tear film of the subject is decreased after the application of the wipe by about: 10%, 15%, 20%, 25%, 30%, 35%, 40%, 45%, 50%, 55%, 60%, 65%, 70%, 75%, or 80% in comparison to the proportion of cholesterol in the total lipid composition of the tear film of the subject before the application of the wipe. In some instances, the proportion of cholesterol in the total lipid composition of the tear film of the subject is decreased by about 35-55% in about 1-42 days after beginning the application of the wipe in comparison to the proportion of cholesterol in the total lipid composition of the tear film of the subject before the application of the wipe. In one instance, the proportion of cholesterol in the total lipid composition of the tear film of the subject is decreased by about 44% in about 21 days after beginning the application of the wipe in comparison to the proportion of cholesterol in the total lipid composition of the tear film of the subject before the application of the wipe. In some instances, the proportion of cholesterol in the total lipid composition of the tear film of the subject is decreased by about 30-50% in about 60-120 days after beginning the application of the wipe in comparison to the proportion of cholesterol in the total lipid composition of the tear film of the subject before the application of the wipe. In one instance, the proportion of cholesterol in the total lipid composition of the tear film of the subject is decreased by about 37% in about 90 days after beginning the application of the wipe in comparison to the proportion of cholesterol in the total lipid composition of the tear film of the subject before the application of the wipe.

In some instances, the proportion of cholesterol in the total lipid composition of the tear film of the subject has a decrease of about 0.1-3% after the application of the wipe in comparison to the proportion of cholesterol in the total lipid composition of the tear film of the subject before the application of the wipe. In some instances, the proportion of cholesterol in the total lipid composition of the tear film of the subject has a decrease of about: 0.1%-0.2%, 0.2%-0.4%, 0.4%-0.6%, 0.6%-0.8%, 0.8%-1.0%, 1%-1.2%, 1.2%-1.4%, 1.4%-1.6%, 1.6%-1.8%, 1.8%-2%, 2%-2.2%, 2.2%-2.4%, 2.4%-2.6%, 2.6%-2.8%, or 2.8%-3% after the application of the wipe in comparison to the proportion of cholesterol in the total lipid composition of the tear film of the subject before the application of the wipe. In some instances, the proportion of cholesterol in the total lipid composition of the tear film of the subject has a decrease of about: 0.4%-0.6%, 0.5%-0.6%, 0.4%-0.7%, 0.5%-0.7%, 0.5%-0.8%, 0.3%-0.8%, or 0.3%-0.7% after the application of the wipe in comparison to the proportion of cholesterol in the total lipid composition of the tear film of the subject before the application of the wipe. In some instances, the proportion of cholesterol in the total lipid composition of the tear film of the subject has a decrease of about: 0.1%, 0.15%, 0.2%, 0.25%, 0.3%, 0.35%, 0.4%, 0.45%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.5%, or 3% after the application of the wipe in comparison to the proportion of cholesterol in the total lipid composition of the tear film of the subject before the application of the wipe. In some instances, the proportion of cholesterol in the total lipid composition of the tear film of the subject has a decrease of about 0.4-0.8% in about 1-42 days after beginning the application of the wipe in comparison to the proportion of cholesterol in the total lipid composition of the tear film of the subject before the application of the wipe. In one instance, the proportion of cholesterol in the total lipid composition of the tear film of the subject has a decrease of about 0.63% in about 21 days after beginning the application of the wipe in comparison to the proportion of cholesterol in the total lipid composition of the tear film of the subject before the application of the wipe. In some instances, the proportion of cholesterol in the total lipid composition of the tear film of the subject has a decrease of about 0.4-0.7% in about 60-120 days after beginning the application of the wipe in comparison to the proportion of cholesterol in the total lipid composition of the tear film of the subject before the application of the wipe. In one instance, the proportion of cholesterol in the total lipid composition of the tear film of the subject has a decrease of about 0.53% in about 90 days after beginning the application of the wipe in comparison to the proportion of cholesterol in the total lipid composition of the tear film of the subject before the application of the wipe.

In one aspect, the ratio of wax ester or cholesterol ester; fatty acids; triglycerides; and cholesterol in the tear film of the subject is about (86 to 91):(13 to 7) : (0.6 to 1.3):(1.5 to 0.8) after the application of the wipe. In some instances, the ratio of wax ester or cholesterol ester; fatty acids; triglycerides; and cholesterol in the tear film of the subject is about (87.8):(10.6):(0.8):(0.8) in about 7-35 days after beginning the application of the wipe. In one instance, the ratio of wax ester or cholesterol ester; fatty acids; triglycerides; and cholesterol in the tear film of the subject is about (87.8):(10.6):(0.8):(0.8) in about 21 days after beginning the application of the wipe. In some instances, the ratio of wax ester or cholesterol ester; fatty acids; triglycerides; and cholesterol in the tear film of the subject is about (90.6):(7.2):(1.3):(0.9) in about 35-120 days after beginning the application of the wipe. In one instance, the ratio of wax ester or cholesterol ester; fatty acids; triglycerides; and cholesterol in the tear film of the subject is about (90.6):(7.2):(1.3):(0.9) in about 90 days after beginning the application of the wipe.

In some instances, the massage is mechanical.

In some instances, the massage is operated by a hand, e.g., a finger, palm, or back of the hand.

In some instances, the massage is conducted to the eye region of the subject with the wipe.

In some instances, the massage is conducted after the heat is generated.

In one aspect, the method further comprises holding the wipe on the eye region of the subject for a period of time. In some instances, the period of time is from about 1 minute to about 15 minutes. In some instances, the period of time is about: 1-2, 2-3, 3-4, 4-5, 5-6, 6-7, 7-8, 8-9, 9-10, 10-11, 11-12, 12-13, 13-14, or 14-15 minutes. In some instances, the period of time is about: 1-5, 2-5, 3-5, 1-10, 2-8, or 4-6 minutes. In some instances, the period of time is about: 1, 2, 3, 4, 5, 6, 7, 8, 9, 10, 11, 12, 13, 14, or 15 minute(s).

In one aspect, the method is repeated one or more times every day or every 2 days. In some instances, the method is repeated at least 2 times per day. In some instances, the method is repeated 1, 2, 3, 4, or 5 times per day. In some instances, the method is repeated once or twice a day. In some instances, method is repeated for about 1-120 days. In some instances, the method is repeated for about: 1-3, 3-7, 7-14, 14-21, 21-28, 28-35, 35-42, 42-49, 49-56, 56-63, 63-70, 70-80, 80-90, 90-100, 100-110, or 110-120 days. In some instances, the method is repeated for about: 1-7, 1-14, 1-21, 1-28, 1-35, 1-42, 1-49, 1-56, 1-63, 1-70, 1-80, 1-90, 1-100, or 1-110 days. In some instances, the method is repeated for about: 3, 7, 14, 21, 28, 35, 42, 49, 56, 63, 70, 80, 90, 100, 110, or 120 days. In some instances, the method is repeated 1-4 times per day for 1-5 weeks and then 1-3 times per day for subsequent 1-10 weeks. In some instances, the method is repeated twice a day for 1-5 weeks and once a day for subsequent 1-10 weeks. In one instance, the method is repeated twice a day for 3 weeks and once a day for the remainder of the three months.

In one aspect, the wipe comprises: a) at least two components adapted to increase a temperature of the wipe from an ambient temperature to a desired temperature upon a reaction of the at least two components, b) a water-impermeable container for preventing the at least two components from coming into contact with an eye when in use, c) a seal for keeping the at least two components separate until a temperature change is initiated, and d) at least one agent selected from the group consisting of soothing agents, cleansing agents, and surfactant agents.

In some instances, the method comprises massaging the eye region of the subject with the wipe after the heat is generated.

In some instances, the at least two components comprise an oxidising agent and a reducing agent.

In some instances, one of the at least two components is a hydratable inorganic salt, e.g., a hydratable sulfate. In some instances, the hydratable sulfate salt is K₂SO₄, Na₂SO₄, Li₂SO₄, MgSO₄, FeSO₄, MnSO₄, CuSO₄, Al₂(SO₄)₃, or ZnSO₄. In one instance, one of the at least two components is MgSO₄. In one instance, the at least two components comprise MgSO₄ and water.

In some instances, the desired temperature is in the range of about 25° C.-56° C. In some instances, the desired temperature is in the range of about 25° C. to about 35° C., about 35° C. to about 40° C., about 40° C. to about 46° C., or about 46° C. to about 53° C.

In some instances, the desired temperature is reached within 15 seconds to 3 minutes after the temperature change is initiated. In some instances, the desired temperature is reached within about 15 seconds to about 30 seconds, about 30 seconds to about 60 seconds, about 1 minute to about 2 minutes, or about 2 minutes to about 3 minutes after the temperature change is initiated. In some instances, the desired temperature is reached in about: 15 seconds, 30 seconds, 45 seconds, about 1 minute, about 2 minutes, or about 3 minutes after the temperature change is initiated.

In some instances, the desired temperature is maintained for from about 5 minutes to about 30 minutes. In some instances, the desired temperature is maintained for at least about: 5, 8, 10, 15, 20, 25, or 30 minutes.

In some instances, the wipe is sterile.

In some instances, the wipe is square, rectangular, circular, ovoid, eye-shaped, curved to correspond to a contour of an eye, sized to be used on one eye region, or sized to be used on two eyes simultaneously.

In some instances, the wipe further comprises a band, a pocket, or a layer for holding the wipe for a period of time. In one instance, the band is elastic.

In some instances, the wipe further comprises an adhesive portion on a part or the whole of one side of the wipe. In one instance, the adhesive portion comprises one or more adhesive materials selected from the group consisting of acrylic adhesives, hydrocolloidal or hydrogel adhesives, natural rubber, synthetic rubber, and any combination thereof.

In some instances, the wipe further comprises a removal strip to protect an adhesive portion. In one instance, the removal strip is a polymer or paper strip treated with silicone.

In some instances, the wipe is coated with a polymeric material capable of moulding to a surface of an eye. In some instances, the wipe is coated with a polymeric material that softens at a temperature above the ambient temperature.

In some instances, the seal is a frangible seal.

In some instances, the at least one agent is at least one cleansing agent or at least one surfactant agent. In some instances, the at least one agent is PEG-80 sorbitan laurate, sodium trideceth sulfate, PEG-150 distearate, cocamidopropyl hydroxysultaine, sodium laureth-13 carboxylate, disodium lauroamphodiacetate, polysorbate 80, polysorbate 20, poloxamer 184, ammonium laureth sulfate, ceteareth 20, ceteareth 25, cocamidopropyl betaine, disodium laureth sulfosuccinate, disodium lauriminodipropionate, disodium lauroamphodipropionate, glycol stearate, hydrogenated castor oil, laureth-23, magnesium laureth, oleth sulfate, PEG-20 stearate, PEG-35 castor oil, PEG-40 hydrogenated castor oil, PEG-60 hydrogenated castor oil, PEG-7 hydrogenated castor oil, PEG-75 lanolin, poloxamer, sodium laureth sulfate, sodium trideceth sulfate, sodium C12-15 pareth 15 sulfonate, sodium C14-16 olefin sulfonate, or any combination thereof.

In some instances, the wipe further comprises a drug delivery system.

In some instances, the wipe further comprises at least one additional agent selected from the group consisting of anti-inflammatory agents, anti-bacterial agents, decongestants, antistatic agents, preservatives, antioxidants, antimicrobial agents, chelating agents, emollients, emulsifying agents, buffering/neutralising agents, humectants, thickeners, viscosity controlling agents, antistatic agents, conditioning agents, and any combination thereof. In one instance, the wipe comprises a viscosity controlling agent that is polyethylene glycol (PEG).

In some instances, the at least one additional agent is incorporated in a thermo-responsive polymer layer coated on a surface of the wipe.

In some instances, the wipe further comprises a reflective layer or a conductive layer to direct heating towards a surface of the wipe.

In some instances, the wipe further comprises an indicator which confirms to a user that the desired temperature is reached. In some instances, the indicator is a temperature sensitive color indicator configured to change from a first color to a second color when the desired temperature is reached. In some instances, the indicator is configured to undergo a reversible color change, such that when the wipe is no longer at the desired temperature or falls outside of a desired temperature range, the indicator reverts to the first color. In some instances, at least a part of the wipe is coated with a temperature reactive ink or a temperature reactive dye.

In one aspect, disclosed herein is a wipe for use in increasing stability of a tear film of a subject, wherein the wipe generates heat and is adapted for massage, and wherein the subject suffers from an eyelid disorder.

In another aspect, disclosed herein is a wipe for use in treating an eyelid disorder in a subject in need thereof, wherein the wipe generates heat and is adapted for massage, and wherein the treating increases stability of a tear film of the subject.

In one aspect, disclosed herein is a wipe for use in modulating a lipid concentration in a tear film of a subject, wherein the wipe generates heat and is adapted for massage, and wherein the subject suffers from an eyelid disorder.

In another aspect, disclosed herein is a wipe for use in treating an eyelid disorder in a subject in need thereof, wherein the wipe generates heat and is adapted for massage, and wherein the treating modulates a lipid concentration in a tear film of the subject.

In one aspect, disclosed herein is a wipe for use in modulating a lipid proportion in a total lipid composition of a tear film of a subject, wherein the wipe generates heat and is adapted for massage, and wherein the subject suffers from an eyelid disorder.

In another aspect, disclosed herein is a wipe for use in treating an eyelid disorder in a subject in need thereof, wherein the wipe generates heat and is adapted for massage, and wherein the treating modulates a lipid proportion in a total lipid composition of the tear film of the subject.

In some instances, the subject herein is a mammal, for example a human.

In some instances, a NIBUT of a tear film is measured by a Tearscope (e.g., a Keeler Tearscope), a slit-lamp (e.g., a Topcon slit-lamp), and/or a digital video capture (e.g., DV3 digital video capture).

In some instances, a lipid concentration is measured by liquid chromatography, e.g., High Performance Liquid Chromatograph (HPLC).

### BRIEF DESCRIPTION OF THE DRAWINGS

FIG. 1 illustrates a circular wipe with heat delivery system.

FIG. 2 illustrates a tubular wipe with heat delivery system.

FIG. 3 illustrates an alternative tubular wipe with heat delivery system;

FIG. 3a illustrates the sides of the tubular wipe of FIG. 3.

FIG. 3b illustrates a modification to the tubular wipe of FIG. 3.

FIG. 4a illustrates an ovoid shaped wipe with an alternative heat delivery system.

FIG. 4b illustrates an alternative arrangement for the ovoid shaped wipe.

FIG. 5 illustrates a folded cloth-like wipe.

FIG. 6 is a graph representing the results of Comparative Example 2.

FIG. 7 is a graph representing the temperature changes noted for the formulation of Example 1.

FIG. 8 is a graph representing the temperature changes noted for the formulation of Example 2.

FIG. 9 is a graph representing the temperature changes noted for the formulation of Example 3.

FIG. 10 is a graph representing the temperature changes noted for the formulation of Example 4.

FIG. 11 is a graph representing the temperature changes noted for the formulation of Example 5.

FIG. 12 is a graph representing the temperature changes noted for the formulation of Example 6.

FIG. 13 is a graph representing the temperature changes noted for the formulation of Example 7.

FIG. 14 is a graph representing the temperature changes noted for the formulation of Examples 8 to 10.

FIG. 15 is a graph representing the temperature changes noted for the formulation of Examples 11 to 13.

FIG. 16 is a graph representing the temperature changes noted for the formulation of Examples 14 to 17.

FIG. 17 is a graph representing the temperature changes noted for the formulation of Examples 18 to 20.

FIG. 18 is a graph representing the temperature changes noted for the formulation of Examples 21 to 30.

FIG. 19 is a graph representing the temperature changes noted for the formulation of Examples 31 to 34.

FIG. 20 is a schematic representation of the wipe of Example 35.

FIG. 21 is a further schematic representation of the wipe of Example 35 at activation.

FIG. 22 is a picture of the water bubbles used in Example 35.

FIG. 23 is a picture of part of a prototype wipe of Example 35.

FIG. 24 is a graph of the temperature profile achieved for the wipe of Example 35.

FIG. 25 is a graph representing the temperature changes noted for the formulations of Examples 36 to 46.

FIG. 26 is a graph representing the temperature changes noted for the formulations of Examples 47 to 49.

FIG. 27 is a picture of uncoated cooling prototype.

FIG. 28 is a graph representing the temperature changes noted for the formulations of Example 50.

FIG. 29 is a schematic representation of the wipe of Example 51.

FIG. 30 is a further schematic representation of the wipe of Example 51 at activation.

FIG. 31 is a picture of an alternative cooling prototype.

FIG. 32 is a graph representing the temperature changes noted for the formulations of Example 51.

FIG. 33 is a graph representing the temperature changes noted for the finger shaped arrangement of Example 52.

FIG. 34 is a line chart showing mean scores of severity of dry eye symptoms in patients in Example 54.

FIG. 35 is a bar chart showing percentage changes in normal, mild, moderate and severe dry eye incidences in Example 54. For the first score group, from left to right are percentages at 7 days, 21 days, and 90 days. For the rest of each score group, from left to right are percentages at enrollment, 7 days, 21 days, and 90 days.

FIG. 36 is a chart showing comfort scores from patients in Example 54.

FIG. 37 is a line chart showing mean increases in tear film stability in patients in Example 54.

FIG. 38 is a line chart showing the total tear film lipid concentrations (μg/μl) in patients' tears in Example 54.

FIG. 39 is a bar chart showing tear concentration percentages (%) of wax esters/cholesterol esters and fatty acids in patients' tear in Example 54. For each group, from left to right are percentages at enrollment, 21 days, and 90 days.

FIG. 40 is a bar chart showing tear concentration percentages (%) of triglycerides and cholesterol in patents' tears in Example 54. For each group, from left to right are percentages at enrollment, 21 days, and 90 days.

### DETAILED DESCRIPTION

The term “tear film”, also known as “lacrimal layer”, “pre-ocular tear film”, “tear layer” or “precorneal film”, generally refers to a composition covering the anterior surface of the cornea which consists of lacrimal fluid and of the secretion of the meibomian and conjunctival glands.

The term “tear film break up time” generally refers to a period from a last blink to the first observance of some instability in tear characteristics. It can be measured by a number of methods including fluorescein staining, or in a non-invasive manner by high-speed videokeratoscopy or with the Tearscope instrument.

The term “fatty acid” refers to a free (unconjugated) carboxylic acid with a long carbon chain (aliphatic tail) of C4 to C30, which is either saturated or unsaturated. Exemplary fatty acids include gadoleic, palmitic, palmitoleic, stearic, oleic, linoleic, arachidic, linolenic, eicosenoic, behenic, erucic, lignoceric, lactic, decate, acetic and myristic fatty acids. Fatty acid chains can be categorized as short to very long by chain length. Short-chain fatty acids refer to fatty acids with aliphatic tails of fewer than six carbons (i.e. butyric acid). Medium-chain fatty acids refer to fatty acids with aliphatic tails of 6-12 carbons, which can form medium-chain triglycerides. Long-chain fatty acids refer to fatty acids with aliphatic tails 13 to 21 carbons. Very long chain fatty acids refer to fatty acids with aliphatic tails longer than 22 carbons.

The term “wax ester” refers to an ester comprised of one molecule of a fatty alcohol esterified with one molecule of fatty acid. Examples of the alcohol component include eicos-11-enol, docos-13-enol, tetracos-15-enol, myristyl alcohol, octyldodecyl stearoyl alcohol, and cetyl alcohol.

The term “cholesterol” refers to a free (unconjugated) sterol having a chemical structure of

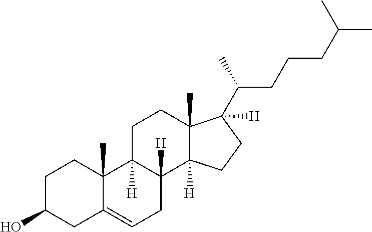

or a stereosiomer thereof.

The term “cholesterol ester”, also known as “cholesteryl ester”, refers to an ester composed of a fatty acid and a cholesterol. Examples of cholesterol esters include cholesteryl oleate, cholesteryl hexanate, cholesteryl palmitate, cholesteryl arachidate, and cholesteryl montanate.

The term “triglyceride” refers to an ester comprised of one molecule of glycerol esterified with three molecules of fatty acids. The three fatty acid components of a triglyceride can be identical or different.

The term “total lipid composition” refers to a total composition of the lipids consisting of wax ester, cholesterol ester, fatty acids (free), triglycerides, and cholesterol (free) in a tear film.

The terms “a” and “an” refers to one or more, unless indicated otherwise.

The term “about” means plus or minus 10% of a referenced numeric indication.

In one aspect, the present disclosure relates to wipes, for example eyelid wipes.

In a further aspect, the present disclosure relates to a method of treatment of disorders of the eyelid margin such as dry eye or those caused by meibomian gland dysfunction.

In one of many aspects, provided herein is a method of treating an eyelid disorder in a subject, comprising applying a wipe to an eye region of the subject, wherein the wipe generates heat and is adapted for massage, and wherein the application of the wipe increases stability of a tear film of the subject, modulates a lipid concentration in a tear film of the subject, and/or modulates a lipid proportion in a total lipid composition of a tear film of the subject. In some instances, the application of the wipe increases stability of a tear film of the subject. In some instances, the application of the wipe modulates a lipid concentration in a tear film of the subject. In some instances, the application of the wipe modulates a lipid proportion in a total lipid composition of a tear film of the subject. In some instances, the application of the wipe increases stability of a tear film of the subject and modulates a lipid concentration in a tear film of the subject. In some instances, the application of the wipe increases stability of a tear film of the subject and modulates a lipid proportion in a total lipid composition of a tear film of the subject. In some instances, the application of the wipe modulates a lipid concentration in a tear film of the subject and modulates a lipid proportion in a total lipid composition of a tear film of the subject. In some instances, the application of the wipe increases stability of a tear film of the subject, modulates a lipid concentration in a tear film of the subject, and modulates a lipid proportion in a total lipid composition of a tear film of the subject.

In one aspect, provided herein is a method of increasing stability of a tear film of a subject, modulating a lipid concentration in a tear film of a subject, and/or modulating a lipid proportion in a total lipid composition of the tear film of a subject, comprising applying a wipe to an eye region of the subject, wherein the wipe generates heat and is adapted for massage, and wherein the subject suffers from an eyelid disorder. In some instances, the method increases stability of a tear film of the subject. In some instances, the method modulates a lipid concentration in a tear film of the subject. In some instances, the method modulates a lipid proportion in a total lipid composition of a tear film of the subject. In some instances, the method increases stability of a tear film of the subject and modulates a lipid concentration in a tear film of the subject. In some instances, the method increases stability of a tear film of the subject and modulates a lipid proportion in a total lipid composition of a tear film of the subject. In some instances, the method modulates a lipid concentration in a tear film of the subject and modulates a lipid proportion in a total lipid composition of a tear film of the subject. In some instances, the method increases stability of a tear film of the subject, modulates a lipid concentration in a tear film of the subject, and modulates a lipid proportion in a total lipid composition of a tear film of the subject.

In some instances, the eyelid disorder is blepharitis, dryness, irritation, redness, inflammation, meibomian gland dysfunction, hay fever, a condition associated with use of eye cosmetics, or a condition associated with contact-lens wear. In one instance, the eyelid disorder is meibomian gland dysfunction.

In one aspect, a non-invasive break-up time (NIBUT) of a tear film of the subject is increased after the application of the wipe in comparison to a NIBUT of a tear film of the subject before the application of the wipe.

In some instances, a NIBUT of a tear film of the subject is increased after the application of the wipe by about: 30-100%, 20-100%, 10-300%, 10-250%, 10-200%, 10-150%, 10-100%, 100-300%, 100-200%, 100-150%, or 100-125% in comparison to a NIBUT of a tear film of the subject before the application of the wipe. In some instances, the NIBUT of the tear film of the subject is increased after the application of the wipe by about 30%-100% in comparison to the NIBUT of the tear film of the subject before the application of the wipe. In some instances, the NIBUT of the tear film of the subject is increased after the application of the wipe by about: 30%-40%, 40%-50%, 50%-60%, 60%-70%, 70%-80%, 80%-90%, or 90%-100% in comparison to the NIBUT of the tear film of the subject before the application of the wipe. In some instances, the NIBUT of the tear film of the subject is increased after the application of the wipe by about: 30%-90%, 40%-80%, 50%-70%, or 55%-65% in comparison to the NIBUT of the tear film of the subject before the application of the wipe. In some instances, the NIBUT of the tear film of the subject is increased after the application of the wipe by about: 30%, 35%, 40%, 45%, 50%, 55%, 60%, 65%, 70%, 75%, 80%, 85%, 90%, 95%, or 100% in comparison to the NIBUT of the tear film of the subject before the application of the wipe. In some instances, the NIBUT of the tear film of the subject is increased by about 55%-65% in about 1-14 days after beginning the application of the wipe in comparison to the NIBUT of the tear film of the subject before the application of the wipe. In one instance, the NIBUT of the tear film of the subject is increased by about 58% in about 7 days after beginning the application of the wipe in comparison to the NIBUT of the tear film of the subject before the application of the wipe. In some instances, the NIBUT of the tear film of the subject is increased by about 60%-70% in about 1-42 days after beginning the application of the wipe in comparison to the NIBUT of the tear film of the subject before the application of the wipe. In one instance, the NIBUT of the tear film of the subject is increased by about 65% in about 21 days after beginning the application of the wipe in comparison to the NIBUT of the tear film of the subject before the application of the wipe.

In some instances, a NIBUT of a tear film of the subject has an increase of about: 1-10, 0.5-10, 0.5-15, 0.5-20, or 0.5-25 seconds after the application of the wipe in comparison to a NIBUT of a tear film of the subject before the application of the wipe. In some instances, the NIBUT of the tear film of the subject has an increase of about 1-10 seconds after the application of the wipe in comparison to the NIBUT of the tear film of the subject before the application of the wipe. In some instances, the NIBUT of the tear film of the subject has an increase of about: 1-2, 2-3, 3-4, 4-5, 5-6, 6-7, 7-8, 8-9, or 9-10 seconds after the application of the wipe in comparison to the NIBUT of the tear film of the subject before the application of the wipe. In some instances, the NIBUT of the tear film of the subject has an increase of about: 1-9, 2-8, 3-7, 4-6, or 4.5-5.5 seconds after the application of the wipe in comparison to the NIBUT of the tear film of the subject before the application of the wipe. In some instances, the NIBUT of the tear film of the subject has an increase of about: 1, 1.5, 2, 2.5, 3, 3.5, 4, 4.5, 5, 5.5, 6, 6.5, 7, 7.5, 8, 8.5, 9, 9.5, or 10 seconds after the application of the wipe in comparison to the NIBUT of the tear film of the subject before the application of the wipe. In some instances, the NIBUT of the tear film of the subject has an increase of about 4-5 seconds in about 1-14 days after beginning the application of the wipe in comparison to the NIBUT of the tear film of the subject before the application of the wipe. In one instance, the NIBUT of the tear film of the subject has an increase of about 4.5 seconds in about 7 days after beginning the application of the wipe in comparison to the NIBUT of the tear film of the subject before the application of the wipe. In some instances, the NIBUT of the tear film of the subject has an increase of about 4-6 seconds in about 1-42 days after beginning the application of the wipe in comparison to the NIBUT of the tear film of the subject before the application of the wipe. In one instance, the NIBUT of the tear film of the subject has an increase of about 5 seconds in about 21 days after beginning the application of the wipe in comparison to the NIBUT of the tear film of the subject before the application of the wipe.

In one aspect, a total lipid concentration in a tear film of the subject is increased after the application of the wipe in comparison to a total lipid concentration of a tear film of the subject before the application of the wipe.

In some instances, a total lipid concentration in a tear film of the subject is increased after the application of the wipe by about: 50-200%, 50-300%, 50-250%, 50-150%, 10-300%, 10-250%, 10-200%, 25-300%, 25-250%, 25-200%, 200-300%, 200-250%, 100-300%, 100-250%, or 100-200% in comparison to a total lipid concentration of a tear film of the subject before the application of the wipe. In some instances, the total lipid concentration in the tear film of the subject is increased after the application of the wipe by about 50%-200% in comparison to the total lipid concentration of the tear film of the subject before the application of the wipe. In some instances, the total lipid concentration in the tear film of the subject is increased after the application of the wipe by about: 50%-60%, 60%-70%, 70%-80%, 80%-90%, 90%-100%, 100%-110%, 110%-120%, 120%-130%, 130%-140%, 140%-150%, 150%-160%, 160-170%, 170%-180%, 180%-190%, or 190%-200% in comparison to the total lipid concentration of the tear film of the subject before the application of the wipe. In some instances, the total lipid concentration in the tear film of the subject is increased after the application of the wipe by about: 50%-100%, 60%-110%, 70%-90%, 100%-160%, 110%-150%, or 120%-140% in comparison to the total lipid concentration of the tear film of the subject before the application of the wipe. In some instances, the total lipid concentration in the tear film of the subject is increased after the application of the wipe by about: 50%, 60%, 70%, 80%, 90%, 100%, 110%, 120%, 130%, 140%, 150%, 160%, 170%, 180%, 190%, or 200% in comparison to the total lipid concentration of the tear film of the subject before the application of the wipe. In some instances, the total lipid concentration in the tear film of the subject is increased by about 75-85% in about 1-42 days after beginning the application of the wipe in comparison to the total lipid concentration of the tear film of the subject before the application of the wipe. In one instance, the total lipid concentration in the tear film of the subject is increased by about 81% in about 21 days after beginning the application of the wipe in comparison to the total lipid concentration of the tear film of the subject before the application of the wipe. In some instances, the total lipid concentration in the tear film of the subject is increased by about 120-140% in about 60-120 days after beginning the application of the wipe in comparison to the total lipid concentration of the tear film of the subject before the application of the wipe. In one instance, the total lipid concentration in the tear film of the subject is increased by about 131% in about 90 days after beginning the application of the wipe in comparison to the total lipid concentration of the tear film of the subject before the application of the wipe.

In some instances, a total lipid concentration in a tear film of the subject has an increase of about: 1-10, 0.5-10, 0.5-15, 0.5-20, or 0.5-25 μg/μl after the application of the wipe in comparison to a total lipid concentration of a tear film of the subject before the application of the wipe. In some instances, the total lipid concentration in the tear film of the subject has an increase of about 1-10 μg/μl after the application of the wipe in comparison to the total lipid concentration of the tear film of the subject before the application of the wipe. In some instances, the total lipid concentration in the tear film of the subject has an increase of about: 1-2 μg/μl, 2-3 μg/μl, 3-4 μg/μl, 4-5 μg/μl, 5-6 μg/μl, 6-7 μg/μl, 7-8 μg/μl, 8-9 μg/μl, or 9-10 μg/μl after the application of the wipe in comparison to the total lipid concentration of the tear film of the subject before the application of the wipe. In some instances, the total lipid concentration in the tear film of the subject has an increase of about: 1-6 μg/μl, 2-5 μg/μl, 2-3.5 μg/μl, or 3.5-5 μg/μl after the application of the wipe in comparison to the total lipid concentration of the tear film of the subject before the application of the wipe. In some instances, the total lipid concentration in the tear film of the subject has an increase of about: 1, 1.5, 2, 2.5, 3, 3.5, 4, 4.5, 5, 5.5, 6, 6.5, 7, 7.5, 8, 8.5, 9, 9.5, or 10 μg/μl after the application of the wipe in comparison to the total lipid concentration of the tear film of the subject before the application of the wipe. In some instances, the total lipid concentration in the tear film of the subject has an increase of about 2-3 μg/μl in about 1-42 days after beginning the application of the wipe in comparison to the total lipid concentration of the tear film of the subject before the application of the wipe. In one instance, the total lipid concentration in the tear film of the subject has an increase of about 2.6 μg/μl in about 21 days after beginning the application of the wipe in comparison to the total lipid concentration of the tear film of the subject before the application of the wipe. In some instances, the total lipid concentration in the tear film of the subject has an increase of about 4-5 μg/μl in about 60-120 days after beginning the application of the wipe in comparison to the total lipid concentration of the tear film of the subject before the application of the wipe. In one instance, the total lipid concentration in the tear film of the subject has an increase of about 4.2 μg/μl in about 90 days after beginning the application of the wipe in comparison to the total lipid concentration of the tear film of the subject before the application of the wipe.

In one aspect, a proportion of wax ester or cholesterol ester in a total lipid composition of a tear film of the subject is increased after the application of the wipe in comparison to a proportion of wax ester or cholesterol ester in a total lipid composition of a tear film of the subject before the application of the wipe.

In some instances, a proportion of wax ester or cholesterol ester in a total lipid composition of a tear film of the subject is increased after the application of the wipe by about: 1-20%, 0.1-20%, 0.5-20%, 0.5-25%, or 0.5-30% in comparison to a proportion of wax ester or cholesterol ester in a total lipid composition of a tear film of the subject before the application of the wipe. In some instances, the proportion of wax ester or cholesterol ester in the total lipid composition of the tear film of the subject is increased after the application of the wipe by about 1%-20% in comparison to the proportion of wax ester or cholesterol ester in the total lipid composition of the tear film of the subject before the application of the wipe. In some instances, the proportion of wax ester or cholesterol ester in the total lipid composition of the tear film of the subject is increased after the application of the wipe by about: 1%-2%, 2%-4%, 4%-6%, 6%-8%, 8%-10%, 10%-12%, 12%-14%, 14%-16%, 16%-18%, or 18%-20% in comparison to the proportion of wax ester or cholesterol ester in the total lipid composition of the tear film of the subject before the application of the wipe. In some instances, the proportion of wax ester or cholesterol ester in the total lipid composition of the tear film of the subject is increased after the application of the wipe by about: 1%-8%, 2%-7%, 3%-6%, 2%-4%, 5%-7%, 4%-8%, or 1%-5% in comparison to the proportion of wax ester or cholesterol ester in the total lipid composition of the tear film of the subject before the application of the wipe. In some instances, the proportion of wax ester or cholesterol ester in the total lipid composition of the tear film of the subject is increased after the application of the wipe by about: 1%, 2%, 3%, 4%, 5%, 6%, 7%, 8%, 9%, 10%, 11%, 12%, 13%, 14%, 15%, 16%, 17%, 18%, 19%, or 20% in comparison to the proportion of wax ester or cholesterol ester in the total lipid composition of the tear film of the subject before the application of the wipe. In some instances, the proportion of wax ester or cholesterol ester in the total lipid composition of the tear film of the subject is increased by about 2-4% in about 1-42 days after beginning the application of the wipe in comparison to the proportion of wax ester or cholesterol ester in the total lipid composition of the tear film of the subject before the application of the wipe. In one instance, the proportion of wax ester or cholesterol ester in the total lipid composition of the tear film of the subject is increased by about 3% in about 21 days after beginning the application of the wipe in comparison to the proportion of wax ester or cholesterol ester in the total lipid composition of the tear film of the subject before the application of the wipe. In some instances, the proportion of wax ester or cholesterol ester in the total lipid composition of the tear film of the subject is increased by about 5-7% in about 60-120 days after beginning the application of the wipe in comparison to the proportion of wax ester or cholesterol ester in the total lipid composition of the tear film of the subject before the application of the wipe. In one instance, the proportion of wax ester or cholesterol ester in the total lipid composition of the tear film of the subject is increased by about 6% in about 90 days after beginning the application of the wipe in comparison to the proportion of wax ester or cholesterol ester in the total lipid composition of the tear film of the subject before the application of the wipe.

In one aspect, a proportion of wax ester and cholesterol ester in a total lipid composition of the tear film of the subject has an increase of about: 1-10%, 0.1-10%, 0.5-10%, 0.5-15%, or 0.5-20% after the application of the wipe in comparison to a proportion of wax ester and cholesterol ester in a total lipid composition of the tear film of the subject before the application of the wipe. In some instances, a proportion of wax ester and cholesterol ester in a total lipid composition of the tear film of the subject has an increase of about 1%-10% after the application of the wipe in comparison to a proportion of wax ester and cholesterol ester in a total lipid composition of the tear film of the subject before the application of the wipe. In some instances, the proportion of wax ester and cholesterol ester in the total lipid composition of the tear film of the subject has an increase of about: 1%-2%, 2%-3%, 3%-4%, 4%-5%, 5%-6%, 6%-7%, 7%-8%, 8%-9%, or 9%-10% after the application of the wipe in comparison to the proportion of wax ester and cholesterol ester in the total lipid composition of the tear film of the subject before the application of the wipe. In some instances, the proportion of wax ester and cholesterol ester in the total lipid composition of the tear film of the subject has an increase of about: 1%-7%, 2-6%, 1.5%-3.5%, or 4.5%-6.5% after the application of the wipe in comparison to the proportion of wax ester and cholesterol ester in the total lipid composition of the tear film of the subject before the application of the wipe. In some instances, the proportion of wax ester and cholesterol ester in the total lipid composition of the tear film of the subject has an increase of about: 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10% after the application of the wipe in comparison to the proportion of wax ester and cholesterol ester in the total lipid composition of the tear film of the subject before the application of the wipe. In some instances, the proportion of wax ester and cholesterol ester in the total lipid composition of the tear film of the subject has an increase of about 2-3% in about 1-42 days after beginning the application of the wipe in comparison to the proportion of wax ester and cholesterol ester in the total lipid composition of the tear film of the subject before the application of the wipe. In one instance, the proportion of wax ester and cholesterol ester in the total lipid composition of the tear film of the subject has an increase of about 2.4% in about 21 days after beginning the application of the wipe in comparison to the proportion of wax ester and cholesterol ester in the total lipid composition of the tear film of the subject before the application of the wipe. In some instances, the proportion of wax ester and cholesterol ester in the total lipid composition of the tear film of the subject has an increase of about 4-6% in about 60-120 days after beginning the application of the wipe in comparison to the proportion of wax ester and cholesterol ester in the total lipid composition of the tear film of the subject before the application of the wipe. In one instance, the proportion of wax ester and cholesterol ester in the total lipid composition of the tear film of the subject has an increase of about 5.2% in about 90 days after beginning the application of the wipe in comparison to the proportion of wax ester and cholesterol ester in the total lipid composition of the tear film of the subject before the application of the wipe.

In one aspect, a proportion of fatty acids in a total lipid composition of a tear film of the subject is decreased after the application of the wipe in comparison to a proportion of fatty acids in the total lipid composition of a tear film of the subject before the application of the wipe.

In some instances, a proportion of fatty acids in a total lipid composition of a tear film of the subject is increased after the application of the wipe by about: 5-80%, 5-90%, 5-100%, 5-150%, 5-200%, 10-90%, 10-100%, 10-150%, or 10-200% in comparison to a proportion of fatty acids in a total lipid composition of a tear film of the subject before the application of the wipe. In some instances, the proportion of fatty acids in the total lipid composition of the tear film of the subject is decreased after the application of the wipe by about 5%-80% in comparison to the proportion of fatty acids in the total lipid composition of the tear film of the subject before the application of the wipe. In some instances, the proportion of fatty acids in the total lipid composition of the tear film of the subject is decreased after the application of the wipe by about 5%-10%, 10%-15%, 15%-20%, 20%-25%, 25%-30%, 30%-35%, 35%-40%, 40%-45%, 45%-50%, 50%-55%, 55%-60%, 65%-70%, 70%-75%, or 75%-80% in comparison to the proportion of fatty acids in the total lipid composition of the tear film of the subject before the application of the wipe. In some instances, the proportion of fatty acids in the total lipid composition of the tear film of the subject is decreased after the application of the wipe by about 5%-60%, 10%-50%, 5%-25%, 5%-30%, 10%-20%, 40%-45%, 30%-50%, 30%-60%, 20%-60%, or 10%-70% in comparison to the proportion of fatty acids in the total lipid composition of the tear film of the subject before the application of the wipe. In some instances, the proportion of fatty acids in the total lipid composition of the tear film of the subject is decreased after the application of the wipe by about: 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, or 80% in comparison to the proportion of fatty acids in the total lipid composition of the tear film of the subject before the application of the wipe. In some instances, the proportion of fatty acids in the total lipid composition of the tear film of the subject is decreased by about 5-30% in about 1-42 days after beginning the application of the wipe in comparison to the proportion of fatty acids in the total lipid composition of the tear film of the subject before the application of the wipe. In one instance, the proportion of fatty acids in the total lipid composition of the tear film of the subject is decreased by about 16% in about 21 days after beginning the application of the wipe in comparison to the proportion of fatty acids in the total lipid composition of the tear film of the subject before the application of the wipe. In some instances, the proportion of fatty acids in the total lipid composition of the tear film of the subject is decreased by about 30-60% in about 60-120 days after beginning the application of the wipe in comparison to the proportion of fatty acids in the total lipid composition of the tear film of the subject before the application of the wipe. In one instance, the proportion of fatty acids in the total lipid composition of the tear film of the subject is decreased by about 43% in about 90 days after beginning the application of the wipe in comparison to the proportion of fatty acids in the total lipid composition of the tear film of the subject before the application of the wipe.

In some instances, a proportion of fatty acids in a total lipid composition of the tear film of the subject has a decrease of about: 1-10%, 0.1-10%, 0.5-10%, 0.5-15%, or 0.5-20% after the application of the wipe in comparison to a proportion of fatty acids in a total lipid composition of the tear film of the subject before the application of the wipe. In some instances, the proportion of fatty acids in the total lipid composition of the tear film of the subject has a decrease of about 1%-10% after the application of the wipe in comparison to the proportion of fatty acids in the total lipid composition of the tear film of the subject before the application of the wipe. In some instances, the proportion of fatty acids in the total lipid composition of the tear film of the subject has a decrease of about: 1%-2%, 2%-3%, 3%-4%, 4%-5%, 5%-6%, 6%-7%, 7%-8%, 8%-9%, or 9%-10% after the application of the wipe in comparison to the proportion of fatty acids in the total lipid composition of the tear film of the subject before the application of the wipe. In some instances, the proportion of fatty acids in the total lipid composition of the tear film of the subject has a decrease of about: 1%-7%, 1%-6%, 1%-3%, 4%-6%, or 4%-7% after the application of the wipe in comparison to the proportion of fatty acids in the total lipid composition of the tear film of the subject before the application of the wipe. In some instances, the proportion of fatty acids in the total lipid composition of the tear film of the subject has a decrease of about: 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10% after the application of the wipe in comparison to the proportion of fatty acids in the total lipid composition of the tear film of the subject before the application of the wipe. In some instances, the proportion of fatty acids in the total lipid composition of the tear film of the subject has a decrease of about 1-3% in about 1-42 days after beginning the application of the wipe in comparison to the proportion of fatty acids in the total lipid composition of the tear film of the subject before the application of the wipe. In one instance, the proportion of fatty acids in the total lipid composition of the tear film of the subject has a decrease of about 2% in about 21 days after beginning the application of the wipe in comparison to the proportion of fatty acids in the total lipid composition of the tear film of the subject before the application of the wipe. In some instances, the proportion of fatty acids in the total lipid composition of the tear film of the subject has a decrease of about 4-7% in about 60-120 days after beginning the application of the wipe in comparison to the proportion of fatty acids in the total lipid composition of the tear film of the subject before the application of the wipe. In one instance, the proportion of fatty acids in the total lipid composition of the tear film of the subject has a decrease of about 5.4% in about 90 days after beginning the application of the wipe in comparison to the proportion of fatty acids in the total lipid composition of the tear film of the subject before the application of the wipe.

In one aspect, a proportion of triglycerides in a total lipid composition of a tear film of the subject is increased after the application of the wipe in comparison to a proportion of triglycerides in a total lipid composition of a tear film of the subject before the application of the wipe.

In some instances, a proportion of triglycerides in a total lipid composition of a tear film of the subject is increased after the application of the wipe by about: 30-200%, 30-250%, 30-300%, 30-150%, 30-100%, 10-300%, 10-250%, 10-200%, 10-150%, 10-100%, 100-300%, 150-300%, 200-300%, or 200-250% in comparison to a proportion of triglycerides in a total lipid composition of a tear film of the subject before the application of the wipe. In some instances, the proportion of triglycerides in the total lipid composition of the tear film of the subject is increased after the application of the wipe by about 30%-200% in comparison to the proportion of triglycerides in the total lipid composition of the tear film of the subject before the application of the wipe. In some instances, the proportion of triglycerides in the total lipid composition of the tear film of the subject is increased after the application of the wipe by about: 30%-40%, 40%-50%, 50%-60%, 60%-70%, 70%-80%, 80%-90%, 90%-100%, 100%-110%, 110%-120%, 120%-130%, 130%-140%, 140%-150%, 150%-160%, 160-170%, 170%-180%, 180%-190%, or 190%-200% in comparison to the proportion of triglycerides in the total lipid composition of the tear film of the subject before the application of the wipe. In some instances, the proportion of triglycerides in the total lipid composition of the tear film of the subject is increased after the application of the wipe by about: 30%-70%, 40%-60%, 130%-190%, 140%-180%, 30%-180%, 40%-170%, or 50%-160% in comparison to the proportion of triglycerides in the total lipid composition of the tear film of the subject before the application of the wipe. In some instances, the proportion of triglycerides in the total lipid composition of the tear film of the subject is increased after the application of the wipe by about: 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, or 200% in comparison to the proportion of triglycerides in the total lipid composition of the tear film of the subject before the application of the wipe. In some instances, the proportion of triglycerides in the total lipid composition of the tear film of the subject is increased by about 40-60% in about 1-42 days after beginning the application of the wipe in comparison to the proportion of triglycerides in the total lipid composition of the tear film of the subject before the application of the wipe. In one instance, the proportion of triglycerides in the total lipid composition of the tear film of the subject is increased by about 51% in about 21 days after beginning the application of the wipe in comparison to the proportion of triglycerides in the total lipid composition of the tear film of the subject before the application of the wipe. In some instances, the proportion of triglycerides in the total lipid composition of the tear film of the subject is increased by about 120%-180% in about 60-120 days after beginning the application of the wipe in comparison to the proportion of triglycerides in the total lipid composition of the tear film of the subject before the application of the wipe. In one instance, the proportion of triglycerides in the total lipid composition of the tear film of the subject is increased by about 151% in about 90 days after beginning the application of the wipe in comparison to the proportion of triglycerides in the total lipid composition of the tear film of the subject before the application of the wipe.

In some instances, the proportion of triglycerides in the total lipid composition of the tear film of the subject has an increase of about: 0.1-3%, 0.01-3%, 0.05-3%, 0.1-4%, 0.01-4%, 0.05-4%, 0.1-5%, 0.01-5%, or 0.05-5% after the application of the wipe in comparison to the proportion of triglycerides in the total lipid composition of the tear film of the subject before the application of the wipe. In some instances, the proportion of triglycerides in the total lipid composition of the tear film of the subject has an increase of about 0.1%-3% after the application of the wipe in comparison to the proportion of triglycerides in the total lipid composition of the tear film of the subject before the application of the wipe. In some instances, the proportion of triglycerides in the total lipid composition of the tear film of the subject has an increase of about: 0.1%-1%, 0.2%-0.9%, 0.2%-0.4%, 0.25%-0.35%, 0.75%-0.85%, 0.7%-0.9%, 0.6%-1.0%, or 0.1%-1.5% after beginning the application of the wipe in comparison to the proportion of triglycerides in the total lipid composition of the tear film of the subject before the application of the wipe. In some instances, the proportion of triglycerides in the total lipid composition of the tear film of the subject has an increase of about: 0.1%-0.2%, 0.2%-0.4%, 0.4%-0.6%, 0.6%-0.8%, 0.8%-1.0%, 1%-1.2%, 1.2%-1.4%, 1.4%-1.6%, 1.6%-1.8%, 1.8%-2%, 2%-2.2%, 2.2%-2.4%, 2.4%-2.6%, 2.6%-2.8%, or 2.8%-3% after beginning the application of the wipe in comparison to the proportion of triglycerides in the total lipid composition of the tear film of the subject before the application of the wipe. In some instances, the proportion of triglycerides in the total lipid composition of the tear film of the subject has an increase of about: 0.1%, 0.15%, 0.2%, 0.25%, 0.3%, 0.35%, 0.4%, 0.45%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.5%, or 3% after the application of the wipe in comparison to the proportion of triglycerides in the total lipid composition of the tear film of the subject before the application of the wipe. In some instances, the proportion of triglycerides in the total lipid composition of the tear film of the subject has an increase of about 0.2-0.4% in about 1-42 days after beginning the application of the wipe in comparison to the proportion of triglycerides in the total lipid composition of the tear film of the subject before the application of the wipe. In one instance, the proportion of triglycerides in the total lipid composition of the tear film of the subject has an increase of about 0.3% in about 21 days after beginning the application of the wipe in comparison to the proportion of triglycerides in the total lipid composition of the tear film of the subject before the application of the wipe. In some instances, the proportion of triglycerides in the total lipid composition of the tear film of the subject has an increase of about 0.6-1.0% in about 60-120 days after beginning the application of the wipe in comparison to the proportion of triglycerides in the total lipid composition of the tear film of the subject before the application of the wipe. In one instance, the proportion of triglycerides in the total lipid composition of the tear film of the subject has an increase of about 0.8% in about 90 days after beginning the application of the wipe in comparison to the proportion of triglycerides in the total lipid composition of the tear film of the subject before the application of the wipe.

In one aspect, a proportion of cholesterol in a total lipid composition of a tear film of the subject is decreased after the application of the wipe in comparison to a proportion of cholesterol in a total lipid composition of a tear film of the subject before the application of the wipe.

In some instances, a proportion of cholesterol in a total lipid composition of a tear film of the subject is increased after the application of the wipe by about: 10-80%, 10-90%, 10-100%, 10-120%, 10-150%, 10-200%, 10-250%, 10-300%, 20-100%, 20-150%, 30-100%, 30-150%, 100-300%, 100-200%, 100-150%, or 100-125% in comparison to a proportion of cholesterol in a total lipid composition of a tear film of the subject before the application of the wipe. In some instances, the proportion of cholesterol in the total lipid composition of the tear film of the subject is decreased after the application of the wipe by about 10%-80% in comparison to the proportion of cholesterol in the total lipid composition of the tear film of the subject before the application of the wipe. In some instances, the proportion of cholesterol in the total lipid composition of the tear film of the subject is decreased after the application of the wipe by about: 10%-15%, 15%-20%, 20%-25%, 25%-30%, 30%-35%, 35%-40%, 40%-45%, 45%-50%, 50%-55%, 55%-60%, 65%-70%, 70%-75%, or 75%-80% in comparison to the proportion of cholesterol in the total lipid composition of the tear film of the subject before the application of the wipe. In some instances, the proportion of cholesterol in the total lipid composition of the tear film of the subject is decreased after the application of the wipe by about: 10%-70%, 20%-60%, or 30%-50% in comparison to the proportion of cholesterol in the total lipid composition of the tear film of the subject before the application of the wipe. In some instances, proportion of cholesterol in the total lipid composition of the tear film of the subject is decreased after the application of the wipe by about: 10%, 15%, 20%, 25%, 30%, 35%, 40%, 45%, 50%, 55%, 60%, 65%, 70%, 75%, or 80% in comparison to the proportion of cholesterol in the total lipid composition of the tear film of the subject before the application of the wipe. In some instances, the proportion of cholesterol in the total lipid composition of the tear film of the subject is decreased by about 35-55% in about 1-42 days after beginning the application of the wipe in comparison to the proportion of cholesterol in the total lipid composition of the tear film of the subject before the application of the wipe. In one instance, the proportion of cholesterol in the total lipid composition of the tear film of the subject is decreased by about 44% in about 21 days after beginning the application of the wipe in comparison to the proportion of cholesterol in the total lipid composition of the tear film of the subject before the application of the wipe. In some instances, the proportion of cholesterol in the total lipid composition of the tear film of the subject is decreased by about 30-50% in about 60-120 days after beginning the application of the wipe in comparison to the proportion of cholesterol in the total lipid composition of the tear film of the subject before the application of the wipe. In one instance, the proportion of cholesterol in the total lipid composition of the tear film of the subject is decreased by about 37% in about 90 days after beginning the application of the wipe in comparison to the proportion of cholesterol in the total lipid composition of the tear film of the subject before the application of the wipe.

In some instances, a proportion of cholesterol in a total lipid composition of a tear film of the subject has a decrease of about: 0.1-3%, 0.01-3%, 0.05-3%, 0.1-4%, 0.01-4%, 0.05-4%, 0.1-5%, 0.01-5%, or 0.05-5% after the application of the wipe in comparison to a proportion of cholesterol in a total lipid composition of a tear film of the subject before the application of the wipe. In some instances, the proportion of cholesterol in the total lipid composition of the tear film of the subject has a decrease of about 0.1-3% after the application of the wipe in comparison to the proportion of cholesterol in the total lipid composition of the tear film of the subject before the application of the wipe. In some instances, the proportion of cholesterol in the total lipid composition of the tear film of the subject has a decrease of about: 0.1%-0.2%, 0.2%-0.4%, 0.4%-0.6%, 0.6%-0.8%, 0.8%-1.0%, 1%-1.2%, 1.2%-1.4%, 1.4%-1.6%, 1.6%-1.8%, 1.8%-2%, 2%-2.2%, 2.2%-2.4%, 2.4%-2.6%, 2.6%-2.8%, or 2.8%-3% after the application of the wipe in comparison to the proportion of cholesterol in the total lipid composition of the tear film of the subject before the application of the wipe. In some instances, the proportion of cholesterol in the total lipid composition of the tear film of the subject has a decrease of about: 0.4%-0.6%, 0.5%-0.6%, 0.4%-0.7%, 0.5%-0.7%, 0.5%-0.8%, 0.3%-0.8%, or 0.3%-0.7% after the application of the wipe in comparison to the proportion of cholesterol in the total lipid composition of the tear film of the subject before the application of the wipe. In some instances, the proportion of cholesterol in the total lipid composition of the tear film of the subject has a decrease of about: 0.1%, 0.15%, 0.2%, 0.25%, 0.3%, 0.35%, 0.4%, 0.45%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.5%, or 3% after the application of the wipe in comparison to the proportion of cholesterol in the total lipid composition of the tear film of the subject before the application of the wipe. In some instances, the proportion of cholesterol in the total lipid composition of the tear film of the subject has a decrease of about 0.4-0.8% in about 1-42 days after beginning the application of the wipe in comparison to the proportion of cholesterol in the total lipid composition of the tear film of the subject before the application of the wipe. In one instance, the proportion of cholesterol in the total lipid composition of the tear film of the subject has a decrease of about 0.63% in about 21 days after beginning the application of the wipe in comparison to the proportion of cholesterol in the total lipid composition of the tear film of the subject before the application of the wipe. In some instances, the proportion of cholesterol in the total lipid composition of the tear film of the subject has a decrease of about 0.4-0.7% in about 60-120 days after beginning the application of the wipe in comparison to the proportion of cholesterol in the total lipid composition of the tear film of the subject before the application of the wipe. In one instance, the proportion of cholesterol in the total lipid composition of the tear film of the subject has a decrease of about 0.53% in about 90 days after beginning the application of the wipe in comparison to the proportion of cholesterol in the total lipid composition of the tear film of the subject before the application of the wipe.

In one aspect, the ratio of wax ester or cholesterol ester; fatty acids; triglycerides; and cholesterol in the tear film of the subject is about (86 to 91):(13 to 7):(0.6 to 1.3):(1.5 to 0.8) after the application of the wipe. In some instances, the ratio of wax ester or cholesterol ester; fatty acids; triglycerides; and cholesterol in the tear film of the subject is about (87.8):(10.6):(0.8):(0.8) in about 7-35 days after beginning the application of the wipe. In one instance, the ratio of wax ester or cholesterol ester; fatty acids; triglycerides; and cholesterol in the tear film of the subject is about (87.8):(10.6):(0.8):(0.8) in about 21 days after beginning the application of the wipe. In some instances, the ratio of wax ester or cholesterol ester; fatty acids; triglycerides; and cholesterol in the tear film of the subject is about (90.6):(7.2):(1.3):(0.9) in about 35-120 days after beginning the application of the wipe. In one instance, the ratio of wax ester or cholesterol ester; fatty acids; triglycerides; and cholesterol in the tear film of the subject is about (90.6):(7.2):(1.3):(0.9) in about 90 days after beginning the application of the wipe.

In some instances, the massage is mechanical.

In some instances, the massage is operated by a hand, e.g., a finger, palm, or back of the hand.

In some instances, the massage is conducted to the eye region of the subject with the wipe.

In some instances, the massage is conducted after the heat is generated.

In one aspect, the method further comprises holding the wipe on the eye region of the subject for a period of time. In some instances, the period of time is from about 1 minute to about 15 minutes. In some instances, the period of time is about: 1-2, 2-3, 3-4, 4-5, 5-6, 6-7, 7-8, 8-9, 9-10, 10-11, 11-12, 12-13, 13-14, or 14-15 minutes. In some instances, the period of time is about: 1-5, 2-5, 3-5, 1-10, 2-8, or 4-6 minutes. In some instances, the period of time is about: 1, 2, 3, 4, 5, 6, 7, 8, 9, 10, 11, 12, 13, 14, or 15 minute(s).

In one aspect, the method is repeated one or more times every day or every 2 days. In some instances, the method is repeated at least 2 times per day. In some instances, the method is repeated 1, 2, 3, 4, or 5 times per day. In some instances, the method is repeated once or twice a day. In some instances, method is repeated for about 1-120 days. In some instances, the method is repeated for about: 1-3, 3-7, 7-14, 14-21, 21-28, 28-35, 35-42, 42-49, 49-56, 56-63, 63-70, 70-80, 80-90, 90-100, 100-110, or 110-120 days. In some instances, the method is repeated for about: 1-7, 1-14, 1-21, 1-28, 1-35, 1-42, 1-49, 1-56, 1-63, 1-70, 1-80, 1-90, 1-100, or 1-110 days. In some instances, the method is repeated for about: 3, 7, 14, 21, 28, 35, 42, 49, 56, 63, 70, 80, 90, 100, 110, or 120 days. In some instances, the method is repeated 1-4 times per day for 1-5 weeks and then 1-3 times per day for subsequent 1-10 weeks. In some instances, the method is repeated twice a day for 1-5 weeks and once a day for subsequent 1-10 weeks. In one instance, the method is repeated twice a day for 3 weeks and once a day for the remainder of the three months.

In one aspect, the wipe comprises: a) at least two components adapted to increase a temperature of the wipe from an ambient temperature to a desired temperature upon a reaction of the at least two components, b) a water-impermeable container for preventing the at least two components from coming into contact with an eye when in use, c) a seal for keeping the at least two components separate until a temperature change is initiated, and d) at least one agent selected from the group consisting of soothing agents, cleansing agents, and surfactant agents.

In some instances, the method comprises massaging the eye region of the subject with the wipe after the heat is generated.

In some instances, the at least two components comprise an oxidising agent and a reducing agent.

In some instances, one of the at least two components is a hydratable inorganic salt, e.g., a hydratable sulfate. In some instances, the hydratable sulfate salt is K₂SO₄, Na₂SO₄, Li₂SO₄, MgSO₄, FeSO₄, MnSO₄, CuSO₄, Al₂(SO₄)₃, or ZnSO₄. In one instance, one of the at least two components is MgSO₄. In one instance, the at least two components comprise MgSO₄ and water.

In some instances, the desired temperature is in the range of about 25° C.-56° C. In some instances, the desired temperature is in the range of about 25° C. to about 35° C., about 35° C. to about 40° C., about 40° C. to about 46° C., or about 46° C. to about 53° C. In some instances, the desired temperature is in the range of about 35° C. to about 46° C., about 35° C. to about 40° C. or about 35° C. to about 53° C. In one instance, the desired temperature is in the range of about 40° C. to about 46° C. In one instance, the desired temperature is in the range of about 42° C. to about 44° C. In one instance, the desired temperature is about 43° C. In some instances, the desired temperature is about: 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., or 53° C.

In some instances, the desired temperature is reached within 5 seconds to 3 minutes after the temperature change is initiated. In some instances, the desired temperature is reached within about 5 seconds to about 30 seconds, about 5 seconds to about 60 seconds, about 10 seconds to about 30 seconds, about 10 seconds to about 60 seconds, about 15 seconds to about 1 minute, about 15 seconds to about 2 minutes, or about 15 seconds to about 3 minutes after the temperature change is initiated. In some instances, the desired temperature is reached within about 5 seconds to about 15 seconds, about 15 seconds to about 30 seconds, about 30 seconds to about 60 seconds, about 1 minute to about 2 minutes, or about 2 minutes to about 3 minutes after the temperature change is initiated. In some instances, the desired temperature is reached in about: 15 seconds, 20 seconds, 25 seconds, 30 seconds, 35 seconds, 40 seconds, 45 seconds, about 1 minute, about 1.5 minutes, about 2 minutes, about 2.5 minutes, or about 3 minutes after the temperature change is initiated.

In some instances, the desired temperature is maintained for from about 5-10, 5-15, 5-20, 5-25, or 5-30 minutes. In some instances, the desired temperature is maintained for at least about: 5, 8, 10, 15, 20, 25, or 30 minutes.

In some instances, the wipe is sterile.

In some instances, the wipe is square, rectangular, circular, ovoid, eye-shaped, curved to correspond to a contour of an eye, sized to be used on one eye region, or sized to be used on two eyes simultaneously.

In some instances, the wipe further comprises a band, a pocket, or a layer for holding the wipe for a period of time. In one instance, the band is elastic.

In some instances, the wipe further comprises an adhesive portion on a part or the whole of one side of the wipe. In one instance, the adhesive portion comprises one or more adhesive materials selected from the group consisting of acrylic adhesives, hydrocolloidal or hydrogel adhesives, natural rubber, synthetic rubber, and any combination thereof.

In some instances, the wipe further comprises a removal strip to protect an adhesive portion. In one instance, the removal strip is a polymer or paper strip treated with silicone.

In some instances, the wipe is coated with a polymeric material capable of moulding to a surface of an eye. In some instances, the wipe is coated with a polymeric material that softens at a temperature above the ambient temperature.

In some instances, the seal is a frangible seal.

In some instances, the at least one agent is at least one cleansing agent or at least one surfactant agent. In some instances, the at least one agent is PEG-80 sorbitan laurate, sodium trideceth sulfate, PEG-150 distearate, cocamidopropyl hydroxysultaine, sodium laureth-13 carboxylate, disodium lauroamphodiacetate, polysorbate 80, polysorbate 20, poloxamer 184, ammonium laureth sulfate, ceteareth 20, ceteareth 25, cocamidopropyl betaine, disodium laureth sulfosuccinate, disodium lauriminodipropionate, disodium lauroamphodipropionate, glycol stearate, hydrogenated castor oil, laureth-23, magnesium laureth, oleth sulfate, PEG-20 stearate, PEG-35 castor oil, PEG-40 hydrogenated castor oil, PEG-60 hydrogenated castor oil, PEG-7 hydrogenated castor oil, PEG-75 lanolin, poloxamer, sodium laureth sulfate, sodium trideceth sulfate, sodium C12-15 pareth 15 sulfonate, sodium C14-16 olefin sulfonate, or any combination thereof.

In some instances, the wipe further comprises a drug delivery system.

In some instances, the wipe further comprises at least one additional agent selected from the group consisting of anti-inflammatory agents, anti-bacterial agents, decongestants, antistatic agents, preservatives, antioxidants, antimicrobial agents, chelating agents, emollients, emulsifying agents, buffering/neutralising agents, humectants, thickeners, viscosity controlling agents, antistatic agents, conditioning agents, and any combination thereof. In one instance, the wipe comprises a viscosity controlling agent that is polyethylene glycol (PEG).

In some instances, the at least one additional agent is incorporated in a thermo-responsive polymer layer coated on a surface of the wipe.

In some instances, the wipe further comprises a reflective layer or a conductive layer to direct heating towards a surface of the wipe.

In some instances, the wipe further comprises an indicator which confirms to a user that the desired temperature is reached. In some instances, the indicator is a temperature sensitive color indicator configured to change from a first color to a second color when the desired temperature is reached. In some instances, the indicator is configured to undergo a reversible color change, such that when the wipe is no longer at the desired temperature or falls outside of a desired temperature range, the indicator reverts to the first color. In some instances, at least a part of the wipe is coated with a temperature reactive ink or a temperature reactive dye.

In one aspect, disclosed herein is a wipe for use in increasing stability of a tear film of a subject, wherein the wipe generates heat and is adapted for massage, and wherein the subject suffers from an eyelid disorder.

In another aspect, disclosed herein is a wipe for use in treating an eyelid disorder in a subject in need thereof, wherein the wipe generates heat and is adapted for massage, and wherein the treating increases stability of a tear film of the subject.

In one aspect, disclosed herein is a wipe for use in modulating a lipid concentration in a tear film of a subject, wherein the wipe generates heat and is adapted for massage, and wherein the subject suffers from an eyelid disorder.

In another aspect, disclosed herein is a wipe for use in treating an eyelid disorder in a subject in need thereof, wherein the wipe generates heat and is adapted for massage, and wherein the treating modulates a lipid concentration in a tear film of the subject.

In one aspect, disclosed herein is a wipe for use in modulating a lipid proportion in a total lipid composition of a tear film of a subject, wherein the wipe generates heat and is adapted for massage, and wherein the subject suffers from an eyelid disorder.

In another aspect, disclosed herein is a wipe for use in treating an eyelid disorder in a subject in need thereof, wherein the wipe generates heat and is adapted for massage, and wherein the treating modulates a lipid proportion in a total lipid composition of the tear film of the subject.

In some instances, the subject herein is a mammal. In some instances, the subject is a dog, cat, monkey, or ape. In some instances, the subject is a human. In some instances, the subject is a child, adult, or elderly.

In some instances, a NIBUT of a tear film is measured by a Tearscope (e.g., a Keeler Tearscope), a slit-lamp (e.g., a Topcon slit-lamp), and/or a digital video capture (e.g., DV3 digital video capture).

In some instances, a lipid concentration is measured by liquid chromatography, e.g., High Performance Liquid Chromatograph (HPLC).

In one aspect, provided herein is a wipe that comprises chemical means for adjusting a temperature of the wipe relative to the ambient temperature.

In one aspect, a function of the wipe is to clean away one or more of: dead cells; debris; meibomian secretions; and the like. In some instances, secretions may be adsorbed onto the surface of the wipe. In some instances, a wipe herein would not be regarded as an absorbent article.

The term “adjusting a temperature” or “adjusting the temperature” means that the temperature of the wipe can change relative to ambient temperature to a pre-determined temperature. The change in temperature may be caused by a heating of the wipe.

The temperature to which the wipe can be adjusted can be dependent on the end-use to which it is to be put. However, it can be understood that by selection of the appropriate chemical means, the required temperature to achieve the desired results can be reproducibly achieved with each wipe thereby obviating the problems associated with the prior art where the cloth may not be adjusted to the correct temperature.

For the treatment of the symptoms of meibomian gland dysfunction, the required temperature is that required to melt the set lipid. As meibomian secretion is usually a mixture of lipids there is normally no sharp melting point and the various lipids present may have melting points over a wide temperature range. For example, a range of from 32° C. to 40° C. with a significant difference in melting points can be noted between normal and abnormal lipid samples. Another example of a temperature range is a range of from 19.5° C. to 32.9° C.

These differences are attributable to the make-up of the secretion. The secretion is generally fluid enough to flow from the glands and spread to form a superficial tear film layer.

In some instances, the temperature adjustment is to be a rise in temperature for example for the treatment of meibomian gland dysfunction, temperatures in the region of from about 40° C. to about 55° C. can generally be desirable with temperatures in the region of from about 45° C. to about 52° C. being desired.

In some instances, the adjusted temperature is maintainable for at least about 5 minutes, e.g., about 8 minutes, about 10 minutes, or more.

In some instances, the temperature adjustment is to be a lowering in temperature for example to treat swelling/edema following trauma temperatures in the range of from about 0° C. to about 25° C. are desirable with temperatures in the range of about 5° C. to about 10° C. being desired. It is desirable that cooling can be maintained from about 5 minutes to about 30 minutes.

To assist the user, the wipe may incorporate an indicator which confirms to the user that the required temperature has been reached. The indicator may be a temperature sensitive colour indicator which can change colour from a first to a second colour when the required temperature is achieved. In one arrangement the colour change can be reversible such that when the wipe is no longer at the desired temperature or has fallen outside the desired temperature range the indicator can revert to the first colour. The colour indicator may be provided by any suitable means. In one arrangement at least a part of the wipe may be coated with a temperature reactive ink or treated with a temperature reactive dye. Thus the user can be advised to wait until a particular colour is achieved before using the wipe and to cease use once the particular colour disappears.

The wipe herein may be of any suitable configuration. In one arrangement it may be a sheet-like material. In one alternative arrangement, the wipe may have increased thickness. The material may be impregnated with or coated with the chemical temperature adjusting means.

The wipe may be a cloth-type material. Where the wipe is formed from a cloth-type material, the cloth may be of any suitable material and may be formed by any technique including weaving, air-laying and the like. Thus the material may be woven or non-woven. The cloth may be made from natural or synthetic fibres or a mixture of both. The material may be selected for its compatibility with the chemical means used for adjusting the temperature of the wipe. Additionally or alternatively, the material may be selected for more aesthetic considerations such as softness and eye-appeal. The cloth may be of any suitable thickness. Suitable thicknesses include those from about 0.2 mm to about 5 mm, for example from about 1 to about 4 mm. However, thicker arrangements may be desirable in some circumstances.

In a second alternative material, the wipe, rather than being formed of a cloth-type material, may be formed from a sponge-type material. The sponge may be a natural or synthetic sponge. The sponge-type material wipe may be of any suitable size and may have a thickness greater than that noted for the cloth-type material wipe.

In a third alternative arrangement, the wipe may be provided as a multi-layered material. The material in each layer may be the same or different. For example, the wipe may comprise a sponge material having attached to one or both surfaces thereof a cloth-type material. Where the cloth-type material is applied to two opposing faces of a sponge-type material, the material used in each face may be the same or different. The multi-layered wipe may be of any suitable thickness.

The wipe may comprise several layers of cloth-type material, each of which may be different. Where a layered structure is used, the temperature adjusting means may be located in or on one or more of the layers.

Where the wipe is to be coated with one or more temperature adjusting materials, it may be coated on one or both sides of the wipe. In some instances, the chemical means is applied to one side to allow an uncoated free side of the wipe. In some arrangements the uncoated side may be applied to the eye. In alternative arrangements, the uncoated side may be used for application of materials such as therapeutic materials, cleaning fluids or the like.

Where the wipe is impregnated with temperature adjusting material, the material may be located throughout the wipe or, for example, may be provided in such a manner that particles of the temperature adjusting material do not come into contact with the eye when in use. The temperature adjusting material may be immobilised in a non-woven pad which is provided, for example, as the cloth-type material or as one layer in a multi-layered arrangement.

In a further alternative arrangement, or where required by the temperature adjusting means selected, the wipe may be an arrangement comprising a pocket or the like into which the temperature adjusting components may be placed, either directly or in a separate container. In an alternative arrangement the material from which the wipe is formed surrounds the temperature adjusting means, for example is wrapped around the temperature adjusting means which may be placed in a suitable container.

The temperature adjusting component may be a self-heating or cooling device or a device relying on external sources of heat or cold. Where external devices are used to cause the temperature adjusting means to change temperature, these may be conventional sources such as ovens, microwave ovens, refrigerators and the like. In one alternative arrangement, the external device may be a purpose-built device. In one arrangement, the chemical temperature adjusting means may be activated by a conducting foil-strip located in the wipe which may be connected to an energy source.

The wipe is single or re-usable. Where multi-use is desirable, the wipe can generally be configured such that it comprises a pocket for containing the temperature adjusting means located in a separate container. On re-use, the temperature adjusting means container can generally be removed and replaced with a fresh container. Where the wipe is to be re-used in this manner, it is produced from washable material.

The wipe may be of any suitable shape and/or configuration. Suitable shapes include squares, rectangles, circles and ovals. In one arrangement, the wipe may be finger-shaped. The wipe may be of any suitable size. It may be sized to be approximately the size of one eye region or may be sized such that it could be used on two eyes simultaneously. In this latter arrangement, a band, such of elastic, may be provided so that the user can wear the wipe for a period of time.

The wipe may be shaped to have a profile which assists its operation. For example, the wipe whether of a square, rectangular, circular or ovoid shape may be curved such that when laid over the closed eye, it can follow the curvature of the eye and ensure that the temperature is provided across the entire eyelid and eyelid margin.

In one arrangement the wipe may be coated, at least on one surface, with a material, such as a polymeric material which is soft such that it moulds to the shape of the users eye and thereby ensures that the temperature is provided across the entire eyelid and eyelid margin. In one arrangement the polymeric material may not be soft at room temperature but softens as the temperature of the wipe increases.

In one arrangement the wipe may include a pocket into which the user may insert one or more fingers. This arrangement can facilitate the user when performing any rubbing of the eyelid margin which may be desirable, for example as part of the treatment of meibomian gland dysfunction. In an arrangement, the wipe may be shaped to correspond to the shape of a finger. One benefit of this arrangement is that the user can be able to readily control the wipe during any rubbing motion.

In one alternative arrangement, the configuration of the wipe may correspond to two or more finger shapes, conjoined, for example by a web of material. In one arrangement, each fingered shaped portion would be arranged to receive at least part of one finger. In one instance, one or more finger portions would include the heating means and one or more other finger portions would be of non-heated fabric which might be used for massaging or for the application of, for example, cleansing agent.

In one arrangement, the wipe, however configured, may include an adhesive portion so that it can be left in place on the eye to warm or cool the eye prior to massaging taking place. In one arrangement, the adhesive portion may comprise at least a part of one side of the wipe. In one alternative arrangement the whole of one side may be adhesive. In this arrangement, the user can normally turn the wipe over prior to massaging/wiping such that massaging/wiping is carried out with a non-adhesive side of the wipe. Where an adhesive portion is used, the wipe can generally be provided with a removal strip, such as a polymer strip or a paper strip surface treated with silicone, in place to protect the adhesive material. Any suitable adhesive material may be used which enable the wipe to be held in place without causing irritation to the delicate eye area and which can enable the wipe to be readily removed without causing discomfort to the user. Examples of adhesive materials include acrylic adhesives, liquid absorbing adhesives, such as a hydrocolloidal or hydrogel adhesives, natural rubber or synthetic rubber.

The adjustment in the temperature of the eyelid wipe may be achieved by any suitable means. However, the selection of the appropriate means can generally be dependent on the required temperature for the wipe and the suitability of any chemical components for use in the delicate eyelid region. The ability of the material to maintain the temperature for the required period of time may also be a factor which can be taken into consideration when selecting the heat adjusting means. Where chemicals are to be used which are not suitable for direct application to the skin or eyelid margin, the arrangement in which the heat adjusting means are placed in a separate container within the wipe can generally be utilised, although any means for separating them from the skin may be used.

In some instances, the method chosen to accomplish the adjustment in temperature enables the wipe to reach the desired temperature in a short period of time, usually less than about 60 seconds. Times in the order of from about 30 to about 60 seconds are desired.

In one arrangement, the wipe, however configured, may alter in temperature on exposure to oxygen, generally the oxygen in the air. Examples include the use of wipes impregnated with, or coated with, material which on exposure to air oxidises and in doing so generates heat. In one arrangement, the material which on exposure to air oxidises to generate heat may be placed in an oxygen-permeable bag which may be enclosed within the wipe or placed in a pocket in the wipe.

Suitable materials include those which form an oxide when reacted with oxygen at room temperature including: iron, aluminum, magnesium, titanium, manganese, zinc, molybdenum and tin oxide (II) with iron powder being desired. The material can generally be provided in powder form to provide a large surface area on which oxidation may occur. The material used may be a mixture of two or more of the foregoing. Further examples of suitable materials include: metal sulphides, polysulphides or hydrosulphides mixed with a catalyst carried on a carbonaceous material; powdered solids such as elemental iron, mixed with salts and water; mixtures of iron powder, water, cellulose, salt and vermiculite activated carbon; iron powder, water, salt and activated charcoal; iron or other metals mixed with alkali metal salts and a catalyst; redox systems such as metal powder (usually ferrous), a metal chloride, water and a water absorber; and alkaline earth metal oxides, such as magnesium oxide, with chlorides or sulfides of alkali metals or alkaline earth metals. It can be understood that some of the components of the mixtures listed do not directly contribute to the heat generating reaction but are present to modify or control the reaction. For example, catalysts, assistants, fillers and moisteners may be present.

Where the temperature adjustment means operates on exposure to air, the wipes can generally be provided to the user in an airtight package. Examples of airtight packages include plastic envelopes, foil, aluminum foil, and pouches. As the package is opened, the wipe can be exposed to air and the adjustment in temperature can commence.

In one alternative arrangement, the wipe, however configured, may alter in temperature on exposure to water. In this arrangement, material impregnated into, coated on, or enclosed within the wipe may be based on chemical substances which generate heat when in the presence of water. The heat generated may be, for example, heat of hydration, dissolution or oxidation. The material which generates heat on contact with water may be placed in a water-permeable bag which may be enclosed within the wipe or placed in a pocket in the wipe.

In use, these wipes would need to be treated with water, or an aqueous solution in order for the heat to be generated. Suitable aqueous solutions include saline, potassium salts, calcium salts, aluminum chloride, calcium chloride, magnesium chloride, potassium sulphate, magnesium sulphate, sodium sulphate and the like. Other solutions may also be used to “wet” the wipe, including those solutions commonly used in contact lens care regimes. The water or other solution may be applied by immersing the wipes in the water or solution or the water/solution may be poured or sprayed onto the wipe.

Suitable materials for altering the temperature of the wipe when contacted with water or other suitable solution include sodium hydroxide, cobalt, chromium, iron, iron hydroxide, magnesium, manganese, molybdenum, tin oxide (II), titanium and calcium hydroxide. These powdered solids may be used alone or in combination. Also suitable are powdered solids such as iron. These may be used alone or with other components such as salt and activated charcoal, or with alkali metals salts and a catalyst. In addition, hydratable organic or inorganic salts such as calcium chloride, calcium sulfate, cerous chloride, sodium carbonate, aluminum chloride, magnesium chloride, magnesium sulfate, zinc citrate, zinc sulfate, zinc nitrates, alkali metal carbonates, alkali metal borates, alkali metal acetates, alkali metal citrates or alkali metal phosphonates may be used. Similarly mixtures such as: anhydrous calcium chloride, cerous chloride, cesium hydroxide, sodium carbonate and organic oxide or salts such as calcium oxide, aluminum chloride or calcium nitrate; sodium chloride with an organic oxide or salt; hypochlorite salts with cellulosic or cellulosic-containing materials; anhydrous calcium chloride and calcium oxide; anhydrous calcium chloride, anhydrous sodium acetate and calcium oxide; boron compounds having a boron-oxygen-boron bond; anhydrous glycol; silica gel; activated alumina; and synthetic zeolites may be used. Anhydrous zeolites, hydratable organic or inorganic salts, magnesium sulphate, magnesium chloride, calcium chloride and calcined gypsum are desired. Each of these would produce an exothermic reaction when mixed with water.

Also suitable are ammonium nitrate, sodium nitrate, ammonium sulphurate, potassium nitrate, sodium thiosulphate, ammonium chloride, ammonium bromide, ammonium iodide, potassium chloride and tin chloride dihydrate which each produce an endothermic reaction when mixed with water.

Whichever method is used to alter the temperature, additional materials may be used to control or extend the reaction.

In a further alternative arrangement, the wipe may contain two components which are kept separate until temperature adjustment is required at which time they are allowed to mix.

This technology may be provided by placing one of the components in a frangible container which may be enclosed within the wipe or, where appropriate, the wipe may comprise a pocket for holding a container comprising one of the components or a container comprising both components separated by any suitable means. In one alternative arrangement, a single container may be used which comprises two or more chambers, each containing one component. When required, the components may be mixed, for example, by the breakage of a frangible seal separating the two chambers such that a reaction between the two components can occur. Where the reaction is an exothermic reaction, heating can occur. Similarly where the reaction is an endothermic reaction, cooling can be achieved.

The container may be made of any suitable material. Where the component to be included in the container is water or an aqueous solution, the material from which the component is manufactured can be water-impermeable. Any suitable water-impermeable material may be used provided that it has the sufficient level of brittleness to rupture when pressure is applied. Suitable materials include polymers such as polyethylene, polypropylene, polyvinyl acetate, polyurethane, silicone rubber and polyvinyl chloride.

The container may be constructed such that the contents can be delivered to the other component in a pre-determined and controlled manner, such that the temperature change can be maintained for the desired period of time. For example, the container may be a flexible container having sealed orifices which open when pressure is applied to the container. The size of the orifices can determine the time period over which the component can be released from the container.

In an alternative arrangement one of the components may be located on the wipe or may be impregnated in the material of the wipe and the other component may be located within a container which is frangible or which has a frangible seal, and which is located within the wipe or within a pocket in the wipe.

In one example of a two component system, the first component may be a compound which generates heat on contact with water, such as calcium oxide, anhydrous magnesium sulphate, colloidal clay or any other of the compounds identified as generating heat on contact with water above and the second component, separated from the first, may be water, saline or other aqueous solution, including surfactant solutions. The second component may additionally include active agents such as anti-inflammatory or anti-bacterial agents.

Other two component systems include: magnesium/iron alloys and electrolytes (suitable electrolytes include saline); magnesium chloride with ethylene glycol; sodium thiosulfate with ethylene glycol; boron-compounds having a boron-oxygen-boron bond with a protic material such as water, methanol, ethanol, propanol, isopropanol, butanol, lower amines, lower alkanol amine, aliphatic oxides and polyols.

In another example of a two component system, the first component may be a compound which cools down on contact with water such as sodium nitrate and the second component separated from the first until activation of the device may be saline, water or other aqueous solutions such as surfactants. The second component may additionally contain active agents such as anti-inflammatory or anti-bacterial agents.

A further example of a two component system is an oxido-reduction system in which an oxidising agent and a reducing agent are used which undergo reaction when combined to generate heat. Examples of oxidising agents include hydrogen peroxide, urea hydrogen peroxide, sodium peroxide, sodium perborate, sodium persulfate, ammonium persulfate and potassium persulfate. Examples of reducing agents include thiourea compounds such as 1-phenyl-2-thio-barbituric acid. Reactions can include: a hydride such as an alkali metal or alkaline earth metal borohydride such as sodium, potassium or calcium borohydrides with an aldehyde such as glyceraldelyde, a ketone such as acetone, a peroxide or a sulfoxide; thiooxypyrimidine or 2-thio-4 oxypyrimidine with hydrogen peroxide or sodium perborate; thiourea with hydrogen peroxide; or alkali metal salts or manganese and chromium oxides such as potassium permanganate or potassium chromate and alcohols or polyols such as glycerin.

A still further example of a two component system includes the reaction between an aqueous salt solution, such as aqueous sodium solution, and seed crystals or metallic triggers that, on contact with the aqueous salt solution, can activate crystallisation and thereby generate heat. Examples include aqueous sodium acetate solution and sodium acetate seed crystals. One benefit of using a system in which crystallisation occurs is that the presence of the resultant crystals within the wipe may be beneficial during massaging of the eye. Some reactions in a two component such as magnesium sulphate and water, a first exothermic reaction can occur followed by a crystallisation which can also generate heat thereby prolonging the heating of the wipe.

Whichever systems are used to adjust the temperature of the wipe, materials may be present to regulate the reaction that causes the adjustment of temperature. Suitable materials include gelling agents, polymers and the like.

It can be understood that some mixtures identified include materials which do not contribute to the heat adjusting reaction but are adjuncts present to control the reaction. Similarly, materials may be present to extend the temperature adjusting reaction. For example, where the heat adjusting reaction is an exothermic reaction, water-containing material that can release water above a particular temperature may be present.

In addition, physical means may be included in the wipe to control the temperature delivered to the user. For example, one or more insulating layers, may be included. Similarly one or more diffusing layers may be incorporated. In one alternative arrangement a covering may be applied to the wipe which assists in the temperature control. The thickness of the one or more layers in the wipe may be selected to assist with temperature control.

Thus, the wipe may include a reflective layer and/or a conductive layer to direct the heating or cooling towards one surface of the wipe. The reflective layer and/or the conductive layer may be made of any suitable material. Examples of suitable materials include metal foils such as aluminium foil. Also suitable are amorphous metallic oxide layers which are very thin and which may be translucent.

The layered wipe including for example diffusion layers, insulating layers and the like and the heat adjusting means located in a container, may have any suitable thickness but can generally be in excess of about 5 mm.

It can be understood that whatever method of heat control is selected, by selection of particular chemical reactions, a required temperature can be achieved and controlled without any decisions having to be taken by the user. In addition the structure of the wipe can be selected to optimise the temperature and control its application to the user.

In some instances, the chemical means may be selected such that the required temperature can be maintained for sufficient time to allow effective treatment to be carried out.

Further, since the reaction can be reproducible in each wipe containing the same temperature adjusting means, the user can be assured of the appropriate temperature to achieve efficacy at each treatment without the risk of burning of the delicate skin in the eye region.

Whilst the present disclosure is described with particular reference to the treatment of meibomian gland dysfunction, it can be understood, that dependent on the temperature of the wipe, there may be a variety of uses. For example, a cooled wipe may be useful in the treatment of the symptoms of, for example, hay fever. The wipes may also be useful in the lid care of contact lens wearers and the sufferers of dry eye and also in the removal of eye cosmetics. In this latter arrangement, in addition to the heat management means, the wipe may include solutions suitable for the removal of eye make-up and/or having lipid solubilising properties.

The eyelid wipe may be provided in a sealed pack formed from any suitable material. Examples of suitable materials include plastics and metal foils. In some instances, the wipe is sterile.

Where the temperature adjusting means is immobilised in the wipe rather than being placed in a container within the wipe or a pocket in the wipe a binder may be used. Suitable binders include cellulose polymers, polyacrylic polymers, polyurethanes polymers, gelatins and gums. Specific examples include hydroxymethylcellulose, hydroxyethylcellulose, hydroxypropylcellulose, cellulose acetates, polyvinylidene and copolymers of polyacrylic acid and polyacrylates.

Whilst the foregoing discussion relates to the temperature adjusting means being located within the wipe, the temperature adjusting means may be located within the packaging in which the wipe is provided. Any of the arrangements detailed herein may be incorporated within the packaging. In use, the user can activate the chemical adjusting means while the wipe is within the packaging, allow the wipe to reach the required temperature and then remove the wipe from the packaging for use. In this arrangement, the wipe can generally be formed from a cloth-type material.

In one arrangement, the eyelid wipe can additionally be impregnated with cleansing agents, surfactant agents or cleansing and surfactant agents. The surfactants may be effective as cleaning agents and/or solubilising agents. Any suitable cleansing or surfactant agents may be used and examples include PEG-80 sorbitan laurate, sodium trideceth sulfate, PEG-150 distearate, cocamidopropyl hydroxysultaine, sodium laureth-13 carboxylate, disodium lauroamphodiacetate, polysorbate 80, polysorbate 20, poloxamer 184, ammonium laureth sulfate, ceteareth 20,25, cocamidopropyl betaine, disodium laureth sulfosuccinate, disodium lauriminodipropionate, disodium lauroamphodipropionate, glycol stearate, hydrogenated castor oil, laureth-23, magnesium laureth, oleth sulfate, PEG-20 stearate, PEG-35 castor oil, PEG-40 hydrogenated castor oil, PEG-60 hydrogenated castor oil, PEG-7 hydrogenated castor oil, PEG-75 lanolin, poloxamers, sodium laureth sulfate, sodium trideceth sulfate, sodium C12-15 pareth 15 sulfonate, and sodium C14-16 olefin sulfonate.

In one aspect, provided herein is a controlled delivery of an appropriate temperature with controlled delivery of agents such as cleaning agents, surfactant agents or soothing agents.

The wipe may also include one or more of antistatic agents, preservatives, antioxidants, antimicrobial agents, chelating agents, emollients, emulsifying agents, buffering/neutralising agents, humectants, thickeners/viscosity controlling agents and antistatic/conditioning agents.

One example of a suitable preservative is imidazolidinyl urea. Suitable antioxidants include tocopherol and tocopheryl acetate. Suitable antimicrobial agents include quaternium-15. EDTA is one example of a suitable chelating agent. Sodium methylparaben, sodium propylparabem and quaternium 8,14 may also be present.

Examples of emollients include natural or mineral oils or esters. Specific examples include potassium C12-13 monoalkyl phosphate polysorbate 60, potassium C12-13 monoalkyl phosphate, calendula officinalis, almond oil PEG-6 esters, capric/caprylic triglyceride, cetearyl alcohol, cocoa butter, decyl oleate, dimethicone, dimethicone copolyol, glyceryl stearate, glyceryl caprylate, glyceryl oleate, glycol stearate, glycol oleate, hydrogenated castor oil, hydrogenated soybean oil, laneth-10 acetate, lanolin, lanolin alcohol, acetylated lanolin alcohol, lecithin, PEG-11 castor oil, PEG-75 lanolin, petrolatum, PPG-26 oleate, PEG-10 butanediol or stearyl alcohol.

Examples of emulsifying agents include PEG-6 caprylic/capric glyceride, ceteareth 20,25, cetearyl alcohol, glycereth-20 stearate, glyceryl stearate, glyceryl caprylate, glyceryl oleate, glycol stearate, glycol oleate, hydrogenated castor oil, laneth-10, laneth 10 acetate, lanolin, lanolin alcohol, laureth-23, lecithin, PEG-20 stearate, PEG-150 distearate, PEG-40 hydrogenated castor oil, PEG 60 hydrogenated castor oil, PEG 7 hydrogenated castor oil, PEG-11 castor oil, PEG-35 castor oil, PEG-15 tallow polyamine, PEG-75 lanolin, poloxamer, polysorbate 20,80, sodium laureth-13 carboxylate, sodium trideceth sulfate and stearic acid.

Examples of buffering/neutralising agents include dipotassium phosphate, sodium hydroxide, potassium phosphate, disodium phosphate, citric acid, aminomethyl propanediol, sodium hydroxide, diethanolamine bisulfate, ethanolamine, hydrochloric acid, lactic acid, sodium phosphate and triethanolamine.

Examples of humectants include propylene glycol, glycereth-20, glycerin, hyaluronic acid, inositol, lactic acid, methyl gluceth-20, PEG-8, PEG-20 stearate, sodium PCA and sorbitol.

Examples of thickeners/viscosity controlling agents such as carbomers, caprylic alcohol, cetearyl alcohol, dextran, disodium lauroamphodiacetate, guar gum hydrogenated castor oil, laneth-10, magnesium sulfate, PEG-150 distearate, stearyl alcohol and xanthan gum.

Examples of antistatic/conditioning agents include dimethicone copolyol, disodium lauriminodipropionate, disodium lauroamphodiacetate, disodium lauroamphodipropionate, glycol oleate, hyaluronic acid, inositol, lanolin, lanolin alcohol, lecithin, panthenol, PEG 15 tallow polyamine, petrolatum, polyquaternium 7,11,16,44 and sodium PCA.

The wipes may additionally or alternatively be impregnated with one or more of the commercially available cleansing agents including those sold under the trade names “Lid-Care” available from CibaVision, “Eye-Scrub” available from Novartis Opthalmics, “Lid Scrub”, “Igiene Daily Eyelid Cleanser” available from Igenics, “Blephasol” available from Laboratoire Thea and “Supranettes” available from Alcon.

Additionally or alternatively, the eyelid wipe may be impregnated with active agents such as anti-inflammatory and anti-bacterial agents and/or decongestants. The use of the combination of heating, optionally also with cleaning, at the time that the active agents are applied to the eyelid, is believed to improve the efficacy of the active agents. It can therefore be understood that in this embodiment, provided herein is a controlled delivery of an appropriate temperature with controlled delivery of the appropriate amount of active agent, optionally with controlled delivery of the required amounts of cleansing solution.

In one arrangement, one or more of cleansing agents, pharmaceutical compositions, active agents and the like may be located in a layer on the outersurface of the wipe which is formed from a thermo-responsive polymer which on heating would soften so that the polymer matrix can reduce the active agent.

In one arrangement, the temperature adjusting means provide cooling and one or more active agents such as anti-inflammatory agents, anti-bacterial agents and/or decongestants are present. Such wipes are useful in the treatment of the symptoms of, for example, hayfever.

The wipe may include, or may be combined with, a drug delivery system such that there is achieved a system which enables the controlled delivery of an appropriate temperature in combination with controlled delivery of a pharmaceutical.

It can be understood that care can be taken in the selection of heat adjusting means and any other components present to ensure that components and products of reaction are not liable to cause damage to the delicate eye region. Where a selection is made where direct contact with the eye region is not advisable, the materials can generally be enclosed within a container in the wipe.

Where the wipe is multi-layered, or where the wipe includes a container for one or more of the components of the chemical heat adjusting means, the edges of the wipe may be sealed either directly or via an intermediate sealing means. Any suitable means of sealing may be used. Suitable means include the use of adhesive, including hot melt and pressure sensitive adhesive, double sided adhesive tape, heat sealing or ultrasonic bonding. In general, the sealing means can be selected such that there is no hard residue which would be uncomfortable when the wipe is in use.

A wipe herein can be used in the treatment of disorders of the eyelid or eyelid margin. In some instances, the wipe is used in the treatment of meibomian gland disorder. In some instances, the wipe is used in the treatment of inflammation of the eyelid.

In a further aspect, provided herein is a method of treating meibomian gland dysfunction and/or blepharitis, the method comprising allowing the wipe of the above first aspect to reach temperature and then massaging the eyelid margin with the wipe. Cleansing agents or the like may be applied to the wipe before massaging is commenced.

In use, it may be desirable for the user to leave the wipe against the eye for a short period prior to commencing massaging of the eyelid margin with the wipe. The period of time required may be of the order of about 5 minutes to about 10 minutes.

In some instances, the wipe herein comprises an excipient. In one instance, the excipient is at least one agent selected from the group consisting of cleansing agents and surfactant agents, such as a surfactant.

In some embodiments, provided herein is a method of treating or ameliorating a disorder of an eyelid or eyelid margin in a subject in need thereof, the method comprising applying a wipe (e.g., eyelid wipe) to an eye region of the subject, whereby the disorder is treated or ameliorated.

In some embodiments, the wipe (e.g., eyelid wipe) delivers heat to the eyelid margin and massages the eyelid.

In some embodiments, the disorder is a dry eye disorder, such as those associated with meibomian gland dysfunction.

In some embodiments, the treating increases the subject's comfort levels.

In some embodiments, the treating or ameliorating increases the subject's comfort levels for all times of a day.

In some embodiments, the treating or ameliorating increases the amount of the subject's tear film lipid secretion.

In some embodiments, the treating or ameliorating increases the total tear film lipid concentration in the subject's tear film lipid secretion.

In some embodiments, the treating or ameliorating increases the concentration of wax esters in the subject's tear film lipid secretion.

In some embodiments, the treating or ameliorating increases the concentration of cholesterol esters in the subject's tear film lipid secretion.

In some embodiments, the treating or ameliorating increases the concentration of triglycerides in the subject's tear film lipid secretion.

In some embodiments, the treating or ameliorating decreases the concentration of fatty acids in the subject's tear film lipid secretion.

In some embodiments, the treating or ameliorating decreases the concentration of cholesterol in the subject's tear film lipid secretion.

In some embodiments, the wipe (e.g., eyelid wipe) further comprises at least one active agent.

In some embodiments, the wipe (e.g., eyelid wipe) further comprises a drug delivery system.

In some embodiments, the wipe (e.g., eyelid wipe) further comprises at least one agent selected from the group consisting of cleansing agents and surfactant agents, such as a surfactant.

In some embodiments, the wipe (e.g., eyelid wipe) further comprises at least one agent selected from the group consisting of antistatic agents, preservatives, antioxidants, antimicrobial agents, chelating agents, emollients, emulsifying agents, buffering/neutralising agents, humectants, thickeners, viscosity controlling agents, antistatic agents, and conditioning agents. In some embodiments, the agent is incorporated in a thermo-responsive polymer layer coated on a surface of the wipe.

In some embodiments, the wipe (e.g., eyelid wipe) further comprises a sealed pack in which the wipe is contained.

In some embodiments, the wipe (e.g., eyelid wipe) is sterile.

In some embodiments, provided herein is a wipe (e.g., eyelid wipe), the wipe comprising a chemical adapted to adjust a temperature of the wipe relative to an ambient temperature.

In some embodiments, the wipe is heated relative to the ambient temperature.

In some embodiments, the temperature is adjusted to a temperature of from about 40° C. to about 55° C.

In some embodiments, the temperature is adjusted to a temperature of from about 45° C. to about 52° C.

In some embodiments, the wipe is cooled relative to the ambient temperature.

In some embodiments, the temperature is adjusted to a temperature of from about 5° C. to about 10° C.

In some embodiments, the temperature is maintained at an adjusted temperature for more than 5 minutes.

In some embodiments, the temperature is maintained at the adjusted temperature for 10 minutes or more.

In some embodiments, the wipe comprises a cloth and wherein the chemical adapted to adjust the temperature of the wipe is coated on the cloth or is impregnated into the cloth.

In some embodiments, the wipe comprises a sponge and wherein the chemical adapted to adjust the temperature of the wipe is coated on or impregnated into the sponge.

In some embodiments, the wipe comprises a plurality of layers and wherein the chemical adapted to adjust the temperature of the wipe is incorporated into at least one of the layers.

In some embodiments, the wipe comprises a pocket into which the chemical adapted to adjust the temperature of the wipe is placed.

In some embodiments, the chemical adapted to adjust the temperature of the wipe is in a container, wherein the container is placed in the pocket.

In some embodiments, the chemical adapted to adjust the temperature of the wipe is activated by an external source.

In some embodiments, the wipe comprises a pocket, wherein the pocket is configured such that, in use, at least one finger can be inserted into the pocket.

In some embodiments, the wipe is finger-shaped.

In some embodiments, the wipe is eye-shaped.

In some embodiments, the wipe comprises to two or more conjoined finger shapes.

In some embodiments, the wipe is curved to correspond to a contour of an eye.

In some embodiments, the wipe is coated with a polymeric material capable of moulding to a surface of the eye.

In some embodiments, the wipe is coated with a polymeric material which softens at a temperature above ambient temperature, such that, in use, the polymeric material is capable of moulding to the surface of the eye.

In some embodiments, the chemical adapted to adjust the temperature of the wipe is activated upon exposure to air.

In some embodiments, the chemical adapted to adjust the temperature of the wipe is activated upon exposure to water or upon exposure to an aqueous solution.

In some embodiments, the chemical adapted to adjust the temperature of the wipe comprises two components capable of reacting with each other to generate a temperature change.

In some embodiments, the reaction of two components is exothermic.

In some embodiments, the two components are MgSO₄ and water.

In some embodiments, the two components are NH₄NO₃ and water.

In some embodiments, the wipe (e.g., eyelid wipe) further comprises at least one active agent.

In some embodiments, the wipe (e.g., eyelid wipe) further comprises a drug delivery system.

In some embodiments, the wipe (e.g., eyelid wipe) further comprises at least one agent selected from the group consisting of cleansing agents and surfactant agents.

In some embodiments, the wipe (e.g., eyelid wipe) further comprises at least one agent selected from the group consisting of antistatic agents, preservatives, antioxidants, antimicrobial agents, chelating agents, emollients, emulsifying agents, buffering/neutralising agents, humectants, thickeners, viscosity controlling agents, antistatic agents, and conditioning agents. In some embodiments, the agent is incorporated in a thermo-responsive polymer layer coated on a surface of the wipe.

In some embodiments, the wipe (e.g., eyelid wipe) further comprises a sealed pack in which the wipe is contained.

In some embodiments, the wipe is sterile.

In some embodiments, provided herein is a method for treating or ameliorating a disorder of an eyelid or a disorder of an eyelid margin, comprising the steps of:

providing a wipe (e.g., eyelid wipe), the wipe comprising a chemical adapted to adjust a temperature of the wipe relative to an ambient temperature;

activating the chemical such that a temperature change is initiated;

allowing the wipe to reach a preselected temperature; and

applying the wipe to an eye region, whereby the disorder of the eyelid or eyelid margin is treated or ameliorated.

In some embodiments, the disorder of the eyelid margin is meibomian gland dysfunction.

In some embodiments, the disorder of the eyelid is inflammation of the eyelid.

In some embodiments, the method herein further comprises the step of: massaging an eyelid margin with the wipe, wherein the step of massaging is conducted after the step of allowing the wipe to reach a preselected temperature.

In some embodiments, the disorder of the eyelid is meibomian gland dysfunction.

In some embodiments, the method herein further comprises the step of holding the wipe on a position on the eye region for a period of time, wherein the step of holding is conducted prior to the step of massaging.

In some embodiments, the period of time is from about 5 minutes to about 10 minutes.

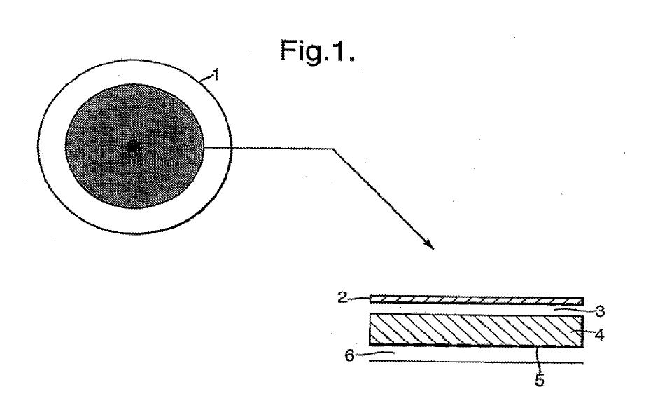

In one embodiment as described in FIG. 1, the wipe 1 is of circular configuration and is formed from a layered structure. The layers present comprise a porous outer layer 2, a layer 3 impregnated with cleaning agent, a heat generating layer 4 comprising material which on exposure to air or water generates heat. This layer is optionally backed by a heat reflective layer 5 which serves to direct the heat released by the heat generating layer 4 towards the outer layer 2. These layers are supported on a holding layer 6 which is generally cloth-like and can form the outer layer which the user can hold in their hand. The wipe 1 can generally be sealed around the edge. The edge margin may comprise solely the porous outer layer 2 and the holding layer 6 bonded together.

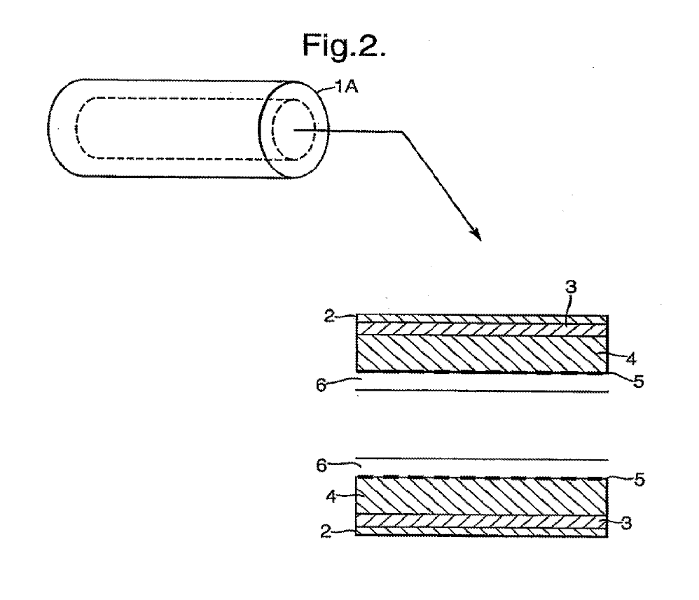

As illustrated in FIG. 2, the wipe 1A may be provided as a tube into which the user may insert a finger to facilitate the massaging step required in use. Here the wipe 1A is formed in a similar manner to the circular wipe of FIG. 1. It can generally be formed as a rectangular material comprising a porous outer layer 2, a layer impregnated with cleaning agent 3, a heat generating layer 4, a heat reflective layer 5 and a holding layer 6. When this rectangle is formed into a tube, the holding layer 6 becomes the inner wall of the tube. In this arrangement, the inner wall may be cloth-like or sponge-like.

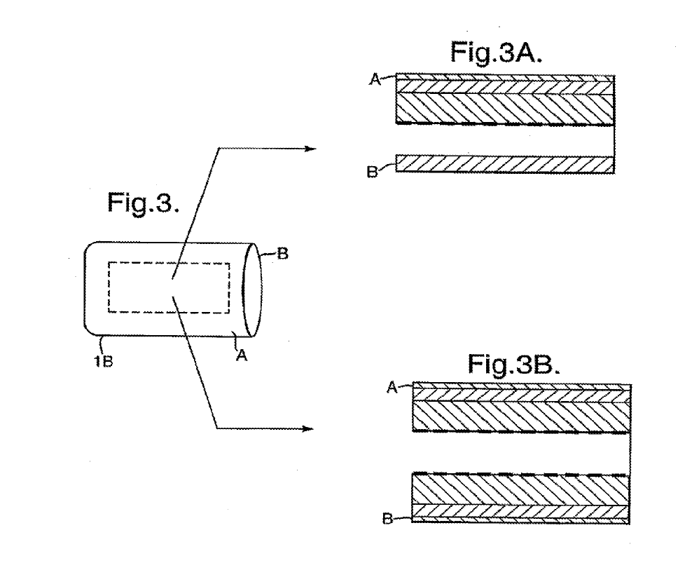

A modification of the tubular wipe is a wipe having a closed end so that the user's finger does not protrude through the wipe. The wipe 1B may have the same configuration throughout the sides of the wipe such as in the tubular arrangement of FIG. 2 as illustrated in FIG. 3B or in the alternative arrangement illustrated in FIG. 3A the wipe may have a side A′ which can, in use, be placed against the eye. The user would normally have this side located on the inside of their finger. The wipe of FIG. 3b can also have a plain holding layer B′ for the exterior of the finger. In this arrangement the side A′ of the finger wipe can have a similar structure to that of the circular wipe of FIG. 1.

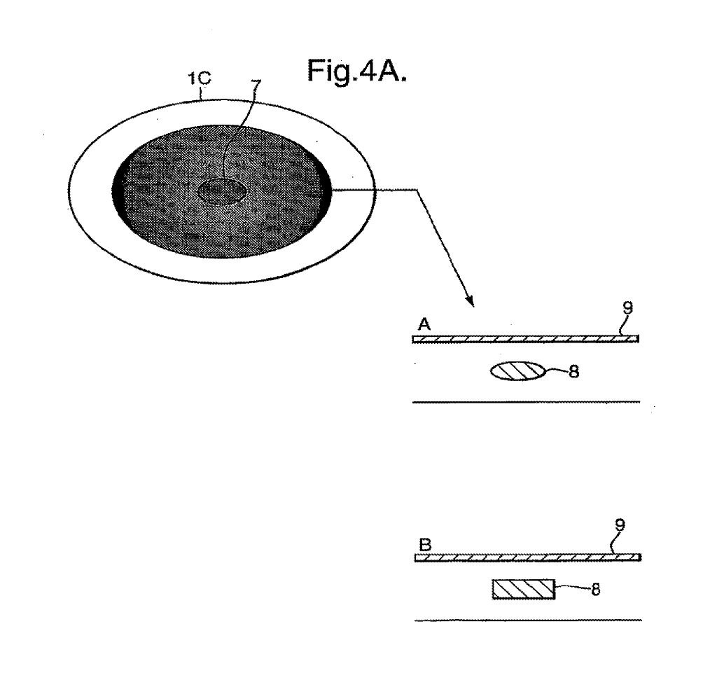

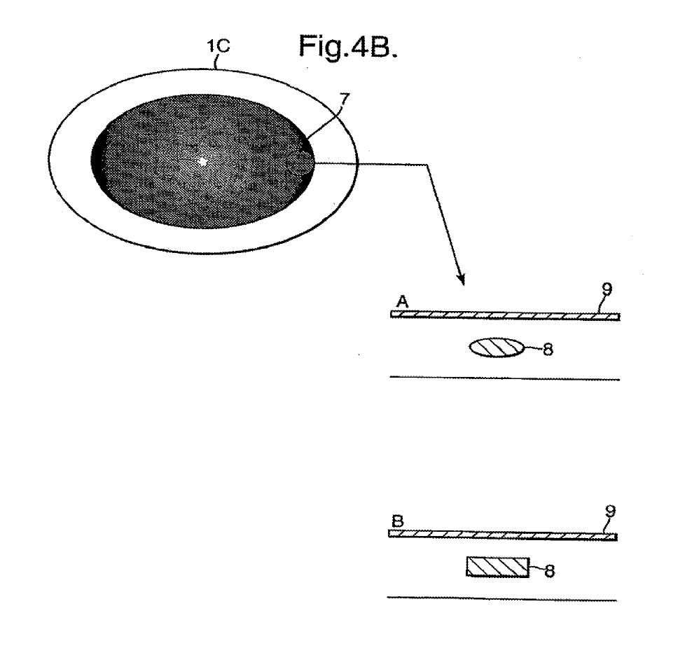

An alternative arrangement is illustrated in FIG. 4a. In this arrangement, the wipe 1C has an ovoid configuration to mirror the overall eye shape. In the centre of the ovoid is a pressure point 7 which when pressed activates the chemical reaction. In this wipe, a two-component means is used to cause the adjustment of temperature. The wipe contains an inner breakable pouch 8 which in A contains seed crystals and in B a metallic trigger. When the user depresses the wipe at the pressure point 7, the pouch breaks such that the seed crystals/metallic trigger come into contact with an aqueous salt solution in which the wipe is first soaked. The wipe of FIG. 4 additionally comprises an optional self-adhesive layer 9. As illustrated in FIG. 4b the pressure point 7 may be located to one side of the wipe.

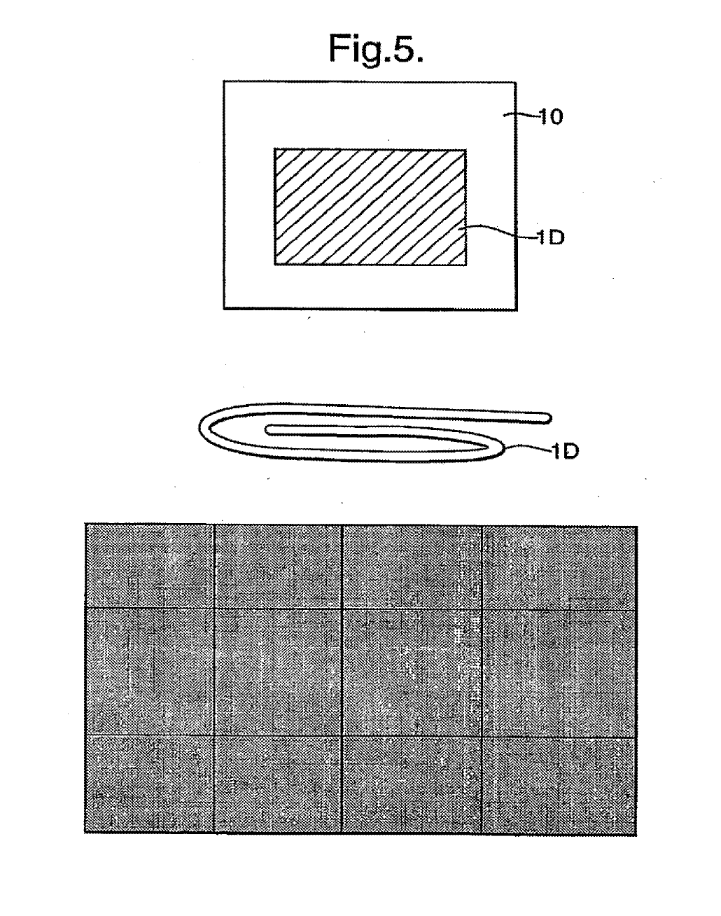

In FIG. 5, the wipe 1D, which on this occasion is a folded cloth-like wipe impregnated with substances which can generate heat when treated with an aqueous solution. Cleaning agents are also impregnated into the wipe. The folded wipe is provided in a moisture impermeable pouch 10.

Some of the above aspects, arrangements, instances, or embodiments can be illustrated by the following examples.

### COMPARATIVE EXAMPLE 1

### Determination of Target Temperature for a Hot Wipe for Use in the Treatment of Meibomian Gland Dysfunction

A cloth was immersed in boiling water and then removed. The temperature of the cloth was measured and the skin tolerance to the cloth was evaluated. The skin tolerance was evaluated by placing the cloth on the inside of the wrist. It was found that the temperature reached was much higher than was acceptable in terms of skin tolerance and that therefore if such a cloth were to be used as a hot compress in the treatment of, for example, meibomian gland dysfunction, there was a risk that injury could occur to the patient. The tolerance level was found to be in the region of 53° C. Temperatures of 43° C. and below were said by the subjects to be not hot enough for any beneficial effect to be felt. The results are summarized in Table 1.

TABLE 1

| WARM CLOTH | Temperature |
|---|---|
| Out of boiling water | approx 63° C. |
| Max tolerable to skin | approx 52-53° C. |
| “Not hot enough” (skin) | approx 43° C. |

### COMPARATIVE EXAMPLE 2

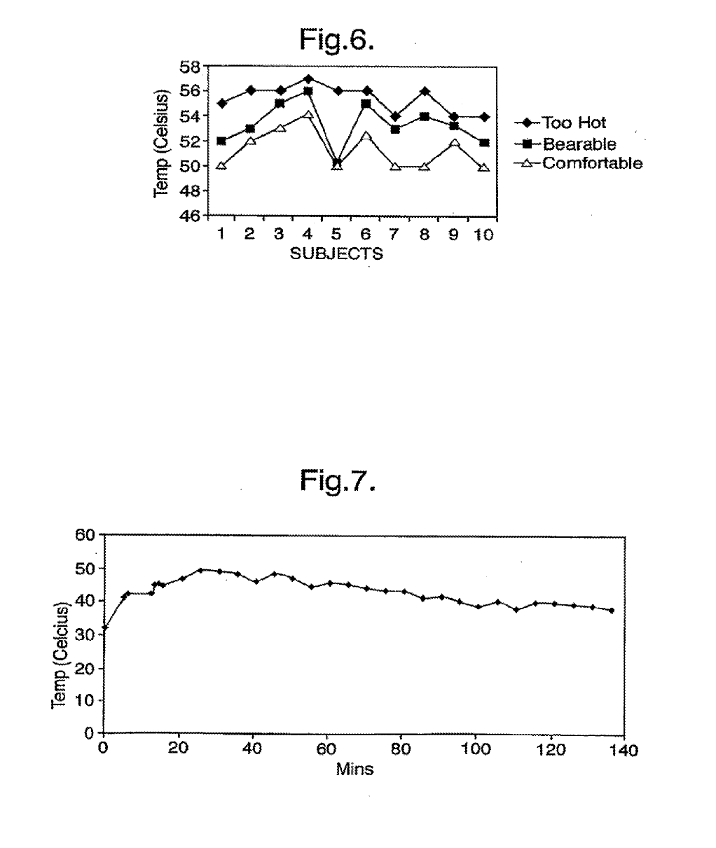

Comparative Example 1 was repeated using a cloth impregnated with heated saline solution. The compress was applied to a closed eye to mimic the routine used by a patient following the conventional treatment for meibomian gland dysfunction. The average results for 10 subjects are summarized in Table 2 below and in the graph of FIG. 6.

TABLE 2

| WARM CLOTH | Temperature |
|---|---|
| “Too hot” | approx >54° C. |
| “Bearable” (eye area) | approx 53° C. |
| “Comfortable” (eye area) | approx 51° C. |

Thus it was noted that a temperature of above 54° C. was uncomfortable. It was often expressed as being too hot for the subjects. A desired temperature was found to be in the region of 51° C. A temperature range of from about 40° -56° C. or 40-46° C. is desired. For maximum efficacy the temperature should be sustained for 10 minutes.

Various formulations for the chemical temperature adjusting means were investigated.

### EXAMPLE 1

### Air Triggered System

A sealed pouch was prepared into which a powder comprising 59% iron powder, 21% water, 10.5% vermiculite, 4% activated charcoal and 5% salt had been placed. The pouch was opened and the temperature changes were noted. These are represented graphically in FIG. 7.

### EXAMPLE 2

### Water Triggered System A

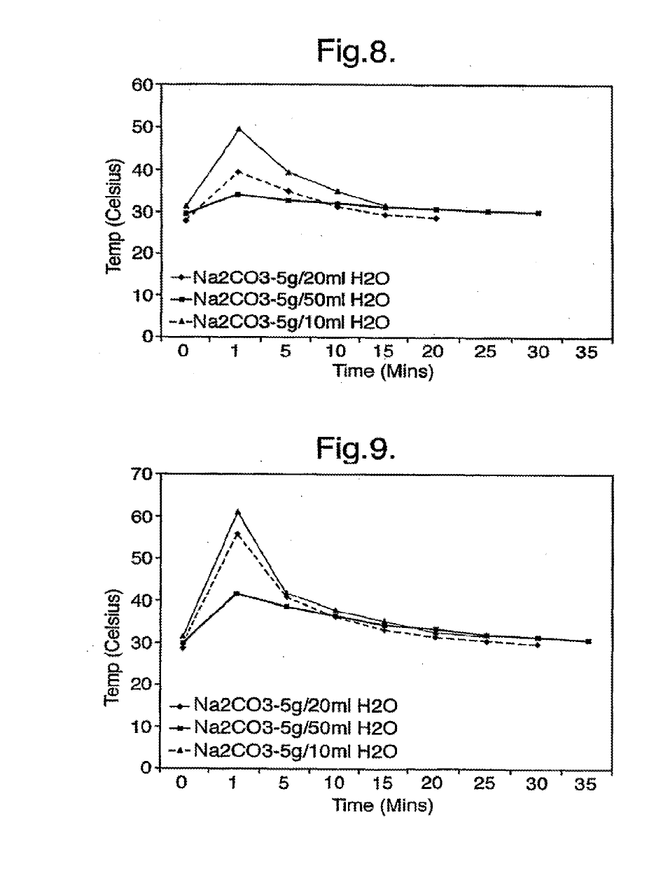

A sealed pouch was prepared into which anhydrous sodium carbonate had been placed. Water was added to the pouch and the temperature changes were noted. These are represented graphically in FIG. 8.

### EXAMPLE 3

### Water Triggered System B

A sealed pouch was prepared into which anhydrous magnesium sulphate had been placed. Water was added to the pouch and the temperature changes were noted. These are represented graphically in FIG. 9.

### EXAMPLE 4

### Water Triggered System C

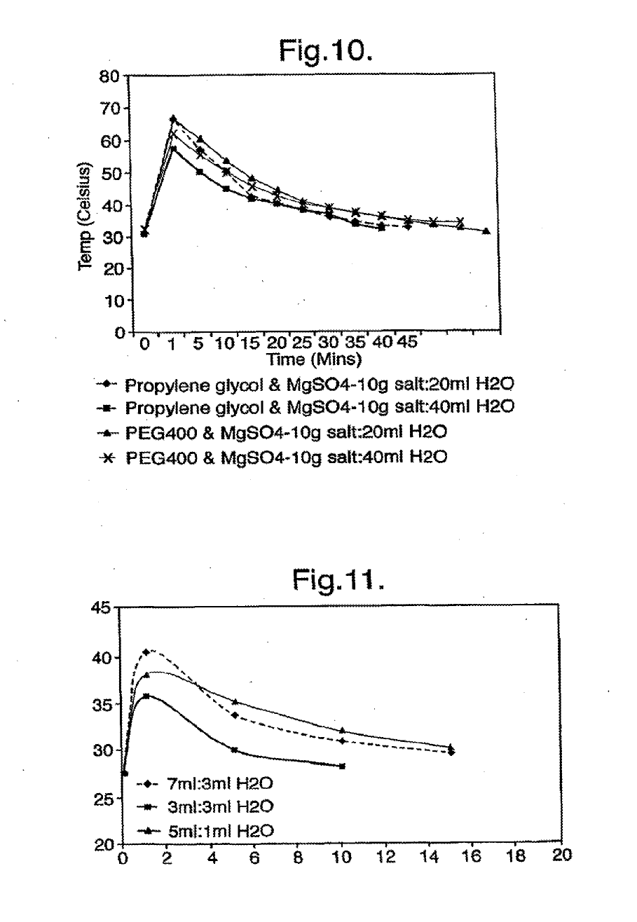

A sealed pouch was prepared into which anhydrous magnesium sulphate (25% w/w) and propylene glycol (75% w/w) or PEG400 (75% w/w) had been placed. Water was added to the pouch and the temperature changes were noted. These are represented graphically in FIG. 10.

### EXAMPLE 5

### Water Triggered System D

A sealed pouch was prepared into which anhydrous sodium potassium aluminosilicate 3 Å (35% w/w), PEG200 (55% w/w) and glycerin (10% w/w) had been placed. Water was added to the pouch and the temperature changes were noted. These are represented graphically in FIG. 11.

### EXAMPLE 6

### Two Component System A

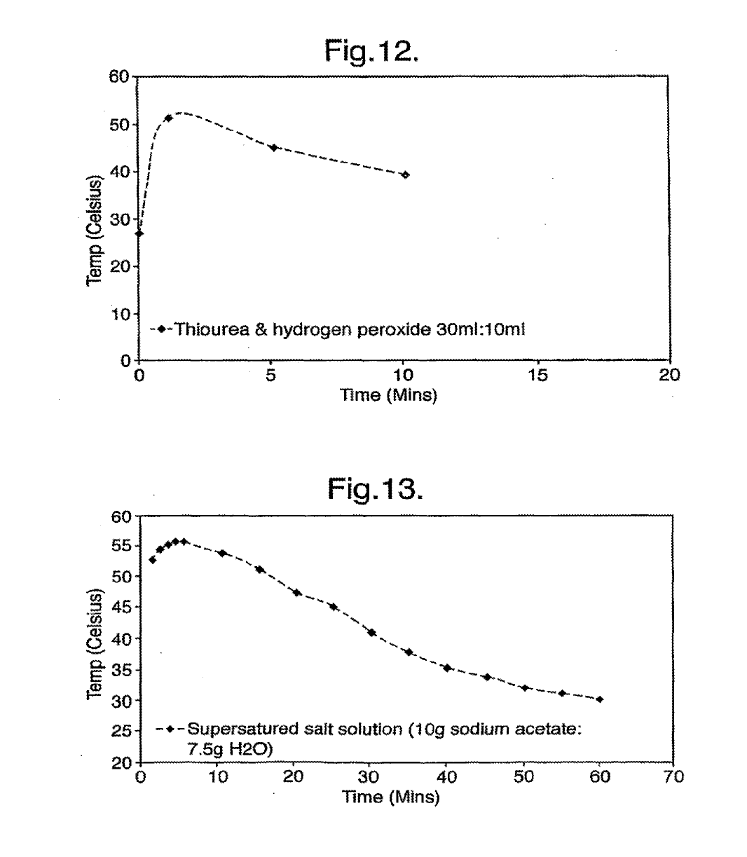

A two component system was prepared. The first component comprised the reducing agent thiourea and the second comprised the oxidation agent hydrogen peroxide solution (approx 8%). The two components were mixed and the temperature changes were measured. These are represented graphically in FIG. 12.

### EXAMPLE 7

### Two Component System B Utilizing a Supersaturated Salt Solution

10 g anhydrous sodium acetate was mixed with 7.5 g water. After the addition of a few salt seed crystals, the temperature changes were measured and are represented in FIG. 13.

### EXAMPLES 8 TO 30

### Optimisation of Temperature Profile

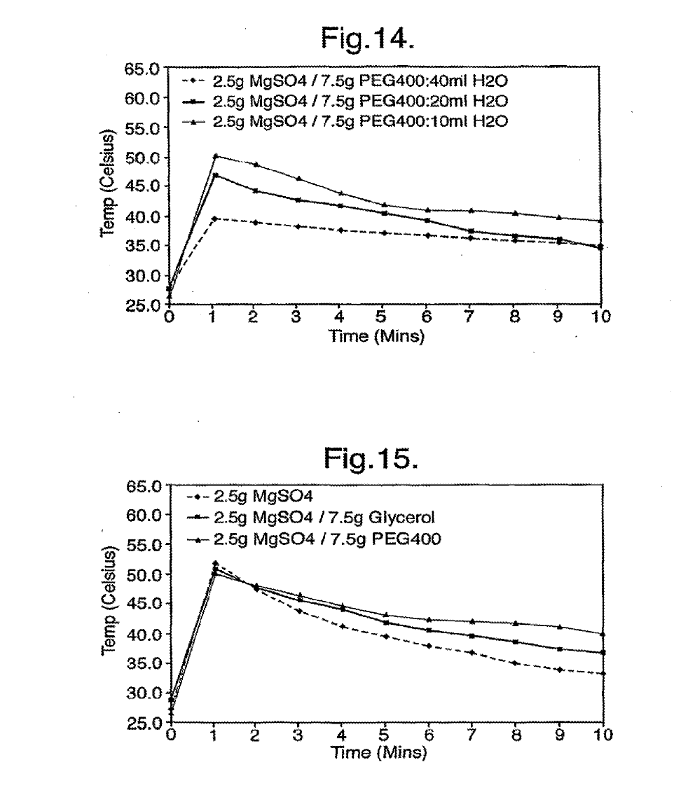

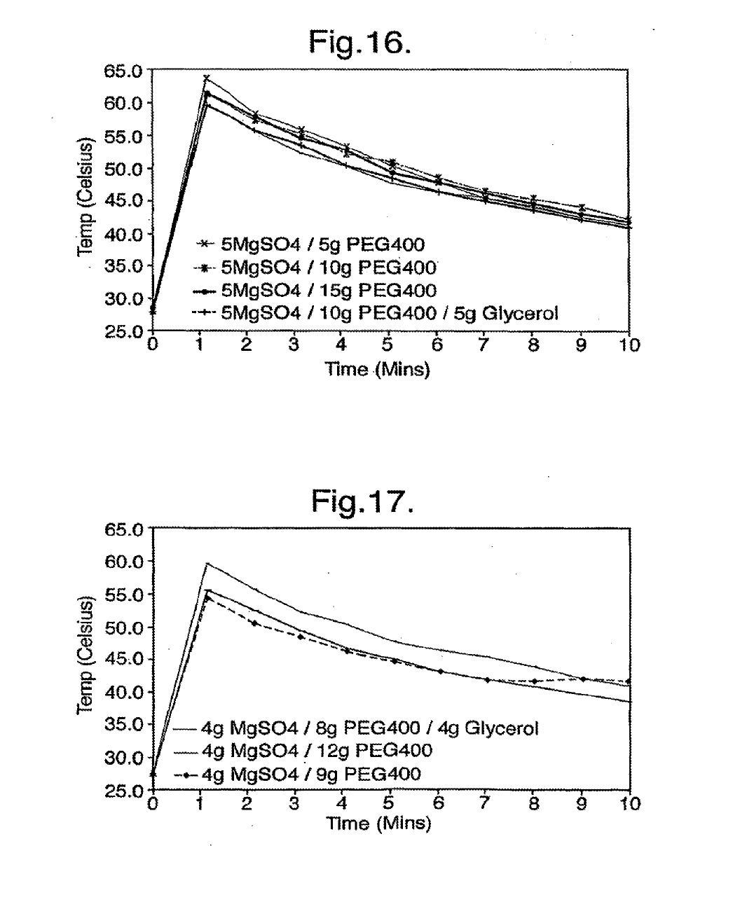

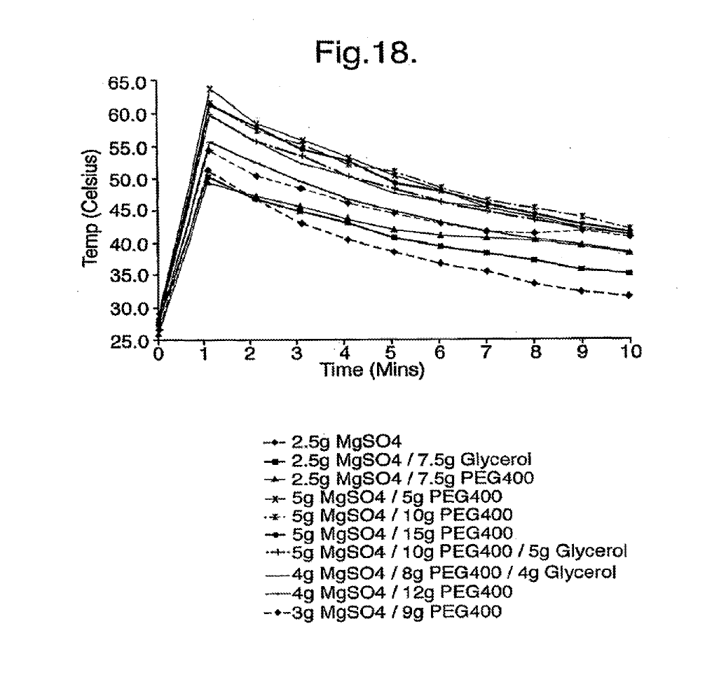

In order to optimize the temperature profile for an inorganic salt/water system various compositions as detailed in Table 3 were investigated. The temperature changes measured are represented graphically in FIGS. 14 to 19.

TABLE 3

| Example | Formulation | FIG. |
|---|---|---|
| 8 | 2.5 g MgSO₄/7.5 g PEG 400:40 ml H₂O | 14 |
| 9 | 2.5 g MgSO₄/7.5 g PEG 400:20 ml H₂O | 14 |
| 10 | 2.5 g MgSO₄/7.5 g PEG 400:10 ml H₂O | 14 |
| 11 | 2.5 g MgSO₄:10 ml H₂O | 15 |
| 12 | 2.5 g MgSO₄/7.5 g glyerol:10 ml H₂O | 15 |
| 13 | 2.5 g MgSO₄/7.5 g PEG 400:10 ml H₂O | 15 |
| 14 | 5 g MgSO₄/5 g PEG 400:10 ml H₂O | 16 |
| 15 | 5 g MgSO₄/10 g PEG 400:10 ml H₂O | 16 |
| 16 | 5 g MgSO₄/15 g PEG 400:10 ml H₂O | 16 |
| 17 | 5 g MgSO₄/10 g PEG 400/5 g glycerol:10 ml H₂O | 16 |
| 18 | 4 g MgSO₄/8 g PEG 400/4 g glycerol:10 ml H₂O | 17 |
| 19 | 4 g MgSO₄/12 g PEG 400:10 ml H₂O | 17 |
| 20 | 3 g MgSO₄/9 g PEG 400:10 ml H₂O | 17 |
| 21 | 2.5 g MgSO₄:10 ml H₂O | 18 |
| 22 | 2.5 g MgSO₄/7.5 g glycerol:10 ml H₂O | 18 |
| 23 | 2.5 g MgSO₄/7.5 g PEG 400:10 ml H₂O | 18 |
| 24 | 5 g MgSO₄/5 g PEG 400:10 ml H₂O | 18 |
| 25 | 5 g MgSO₄/10 g PEG 400:10 ml H₂O | 18 |
| 26 | 5 g MgSO₄/15 g PEG 400:10 ml H₂O | 18 |
| 27 | 5 g MgSO₄/10 g PEG 400/5 g glycerol:10 ml H₂O | 18 |
| 28 | 4 g MgSO₄/8 g PEG 400/4 g glycerol:10 ml H₂O | 18 |
| 29 | 4 g MgSO₄/12 g PEG 400:10 ml H₂O | 18 |
| 30 | 3 g MgSO₄/9 g PEG 400:10 ml H₂O | 18 |

For the formulations of Examples 23 and 30 the heat generated was better sustained between 7 and 10 minutes post activation and therefore offered advantages when compared with the other formulations.

### EXAMPLES 31 TO 34

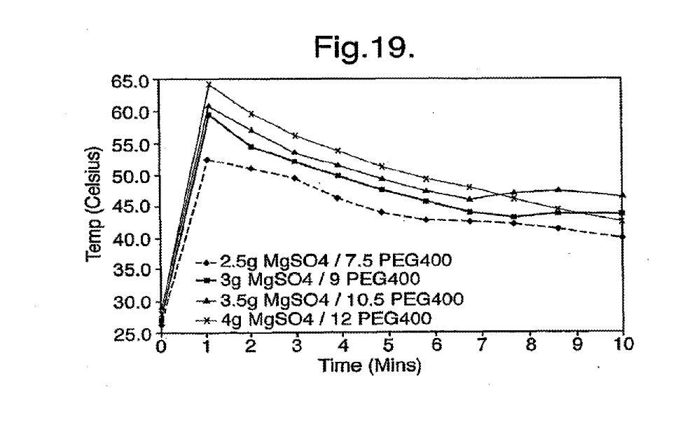

Further tests were carried out to optimize the salt mixture system with MgSO₄ from 2.5 g to 4 g. The details are set out in Table 4 and the temperature profile is represented graphically in FIG. 19.

TABLE 4

| Example | Formulation | FIG. |
|---|---|---|
| 31 | 2.5 g MgSO₄/7.5 g PEG 400:10 ml H₂O | 19 |
| 32 | 3 g MgSO₄/9 g PEG 400:10 ml H₂O | 19 |
| 33 | 3.5 g MgSO₄/10.5 g PEG 400:10 ml H₂O | 19 |
| 34 | 4 g MgSO₄/12 g PEG 400:10 ml H₂O | 19 |

The formulation of Example 33 sustained a temperature of just below 45° C. after 7 minutes and was therefore chosen for use in Example 35 in the production of a prototype wipe.

### EXAMPLE 35

### Prototype Heating Wipe

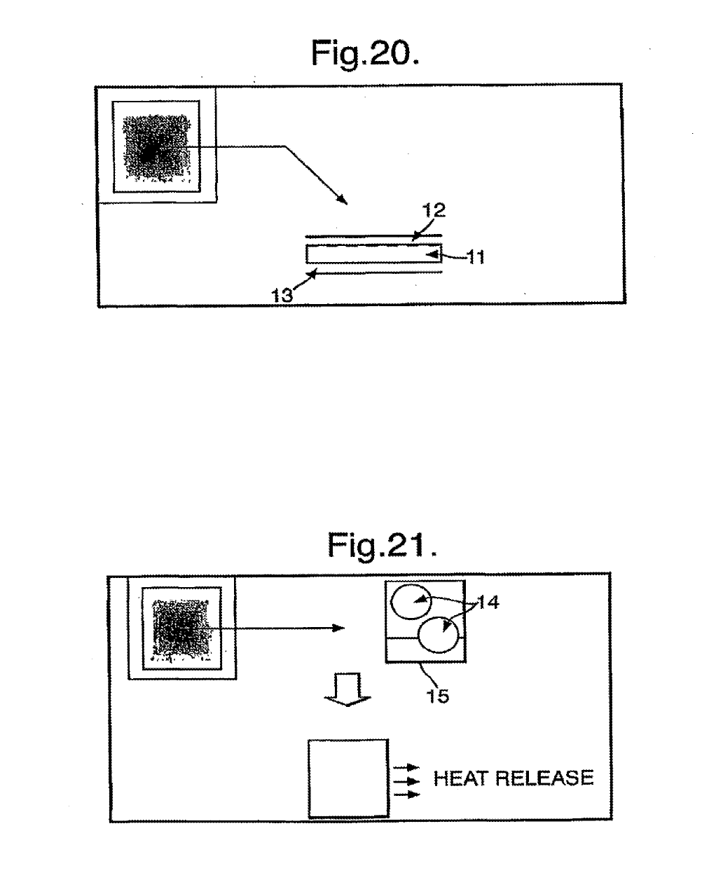

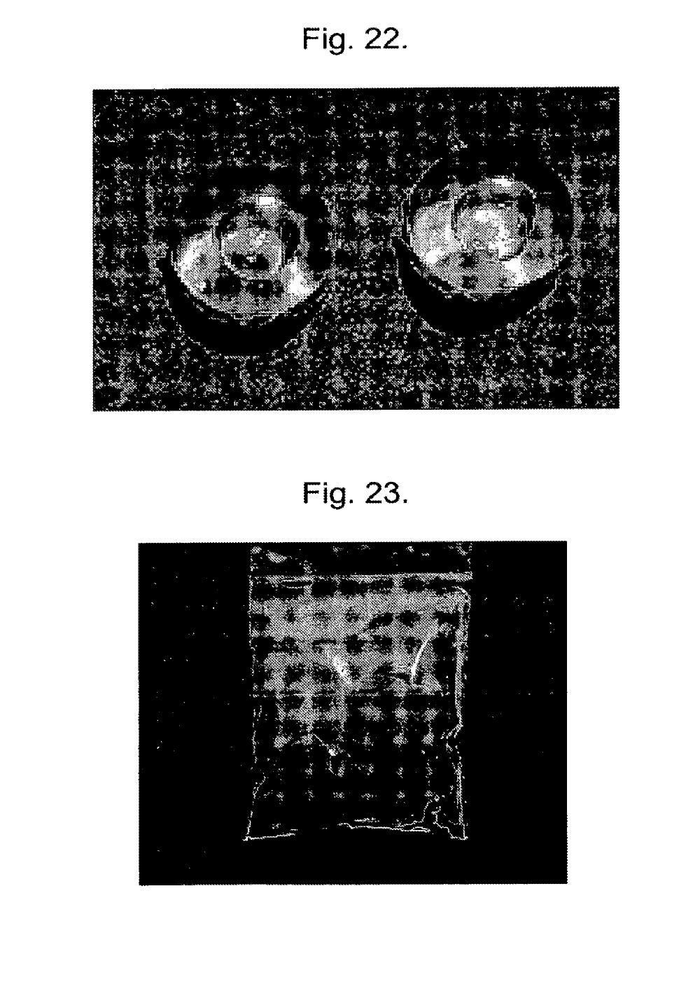

A prototype was produced based on a water triggered system which was activated by pressure. The heat produced was the result of the exothermic reaction of the inorganic salt mixture with water. The water was provided as a water bubble within a frangible plastic envelope of polyethylene as illustrated in FIG. 22. Two water bubbles were used each containing 5 ml water. The water bubbles and the salt mixture of 3.5 g MgSO₄/10.5 g PEG 400 were inserted in a sealable water-tight pouch to form the heat-generating compartment 11 of FIG. 20. The heat generating compartment 11 is coated on one side 12 with a sheet of aluminium foil covered with gauze and on the other side 13 with a cotton pad. In use it is the side 12 which is applied to the closed eyelids for the heat treatment. After heat treatment side 13 can be impregnated with cleaning solution and used to wipe the lid margin. The pad on side 13 may, in an alternative arrangement, be pre-impregnated with the cleaning solution. A picture of the prototype wipe is in FIG. 23. In production the wipe can generally be of an optimum size and shape for use by the user.

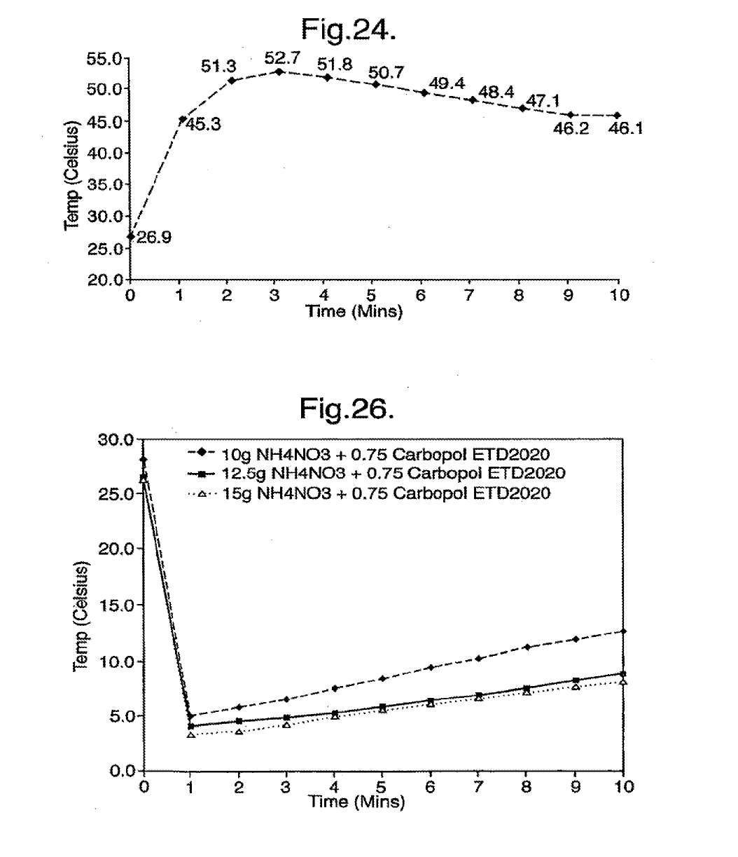

The operation of wipe is illustrated schematically in FIG. 21. Gentle pressure on the external surfaces of the wipe causes the water bubbles 14 to burst so that the water comes into contact with the inorganic salt mixture 15 so that heat is released. The temperature profile is illustrated graphically in FIG. 24. As indicated above, a temperature of from 45 to 52° C. is desirable. The wipe reached the required temperature within 1 minute of activation and the temperature was maintained for 10 minutes. Crystallization occurred.

### EXAMPLE 36 TO 46

### Water Triggered System for Cooling

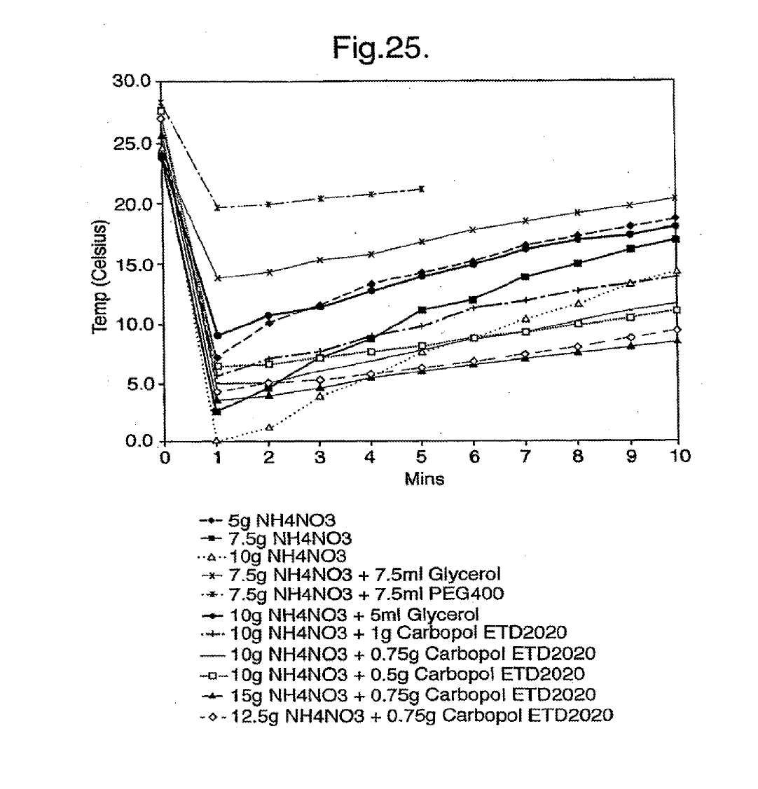

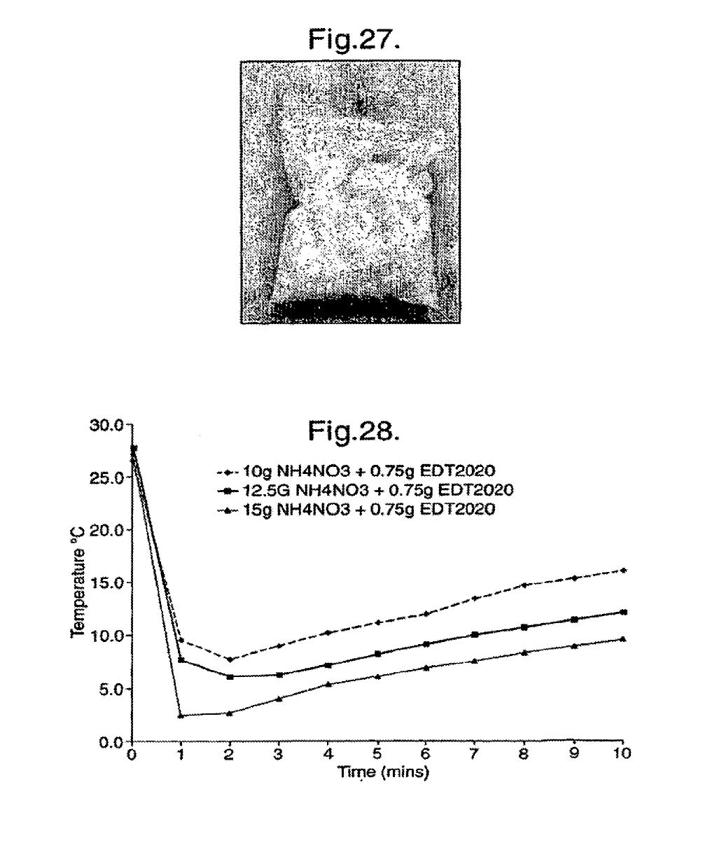

Various compositions were prepared as detailed in Table 5 and the temperature profiles on the addition of water were measured. The results are illustrated graphically in FIG. 25.

TABLE 5

| Example | Formulation | FIG. |
|---|---|---|
| 36 | 5 g NH₄NO₃:10 ml H₂O | 25 |
| 37 | 7.5 g NH₄NO₃:10 ml H₂O | 25 |
| 38 | 10 g NH₄NO₃:10 ml H₂O | 25 |
| 39 | 7.5 g NH₄NO₃/7.5 ml glycerol:10 ml H₂O | 25 |
| 40 | 7.5 g NH₄NO₃/7.5 ml PEG400:10 ml H₂O | 25 |
| 41 | 10 g NH₄NO₃/5 ml glycerol:10 ml H₂O | 25 |
| 42 | 10 g NH₄NO₃/1 g | 25 |
| | Carbopol ETD2020:10 ml H₂O | |
| 43 | 10 g NH₄NO₃/0.75 g | 25 |
| | Carbopol ETD2020:10 ml H₂O | |
| 44 | 10 g NH₄NO₃/0.5 g | 25 |
| | Carbopol ETD2020:10 ml H₂O | |
| 45 | 15 g NH₄NO₃/0.75 g | 25 |
| | Carbopol ETD2020:10 ml H₂O | |
| 46 | 12.5 g NH₄NO₃/0.75 g | 25 |
| | Carbopol ETD2020:10 ml H₂O | |
| 47 | 10 g NH₄NO₃/0.75 g | 26 |
| | Carbopol ETD2020:10 ml H₂O | |
| 48 | 12.5 g NH₄NO₃/0.75 g | 26 |
| | Carbopol ETD2020:10 ml H₂O | |
| 49 | 15 g NH₄NO₃/0.75 g | 26 |
| | Carbopol ETD2020:10 ml H₂O | |

### EXAMPLE 50

### Prototype Cooling Wipe

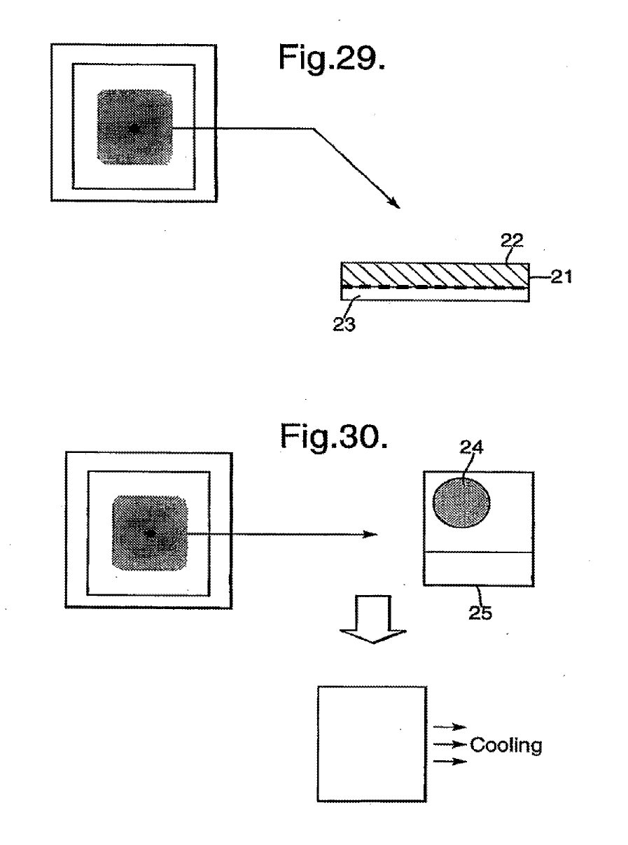

A prototype was produced based on a water triggered system which was activated by pressure. The cold produced was the result of the endothermic reaction of the ammonium nitrate with water. The water was provided as a water bubble within a frangible plastic envelope of polyethylene as illustrated in FIG. 22. One water bubble was used containing 10 ml water. The water bubble and the salt mixture of 10 g to 15 g NH₄NO3/0.75 g ETD2020 (as detailed in Table 6) were inserted in a sealable water-tight pouch to form the cold-generating compartment 21 of FIG. 29. In this example, the cold generating compartment 21 was left uncoated. Pressure was applied to the water bubble such that the water mixed with the ammonium nitrate and cold was generated. The temperature profiles are illustrated graphically in FIG. 28.

TABLE 6

| Run | Formulation | FIG. |
|---|---|---|
| A | 10 g NH₄NO₃/0.75 g ETD2020:10 ml H₂O | 28 |
| B | 12.5 g NH₄NO₃/0.75 g ETD2020:10 ml H₂O | 28 |
| C | 15 g NH₄NO₃/0.75 g ETD2020:10 ml H₂O | 28 |

### EXAMPLE 51

### Prototype Cooling Wipe 2

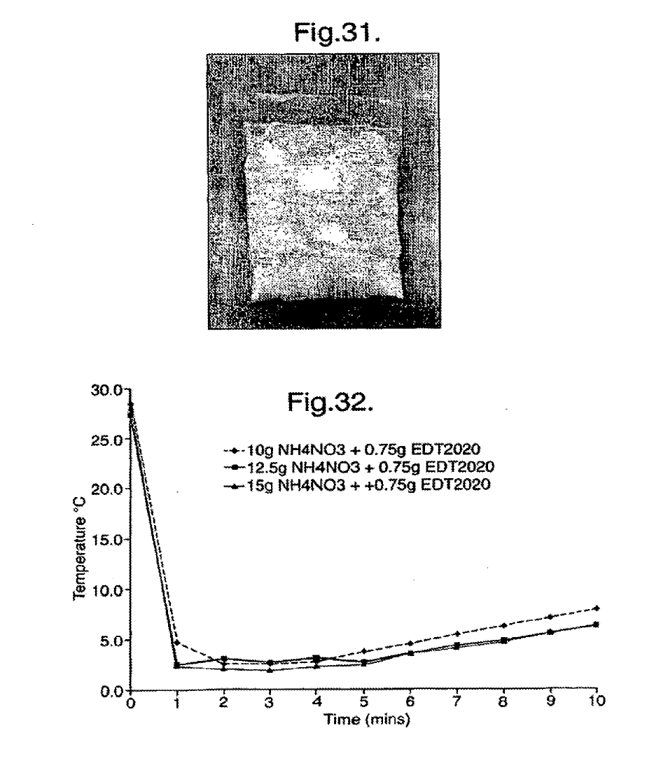

Example 51 was repeated except that the wipe was left uncoated on one side 22 and was coated on the other side 23 with a cotton pad. In use it is the side 22 which is to be applied to the closed eyelids for the cooling treatment. After cooling treatment side 23 can be impregnated with any applicable treatment solution and used to wipe the lid margin. The pad on side 23 may be pre-impregnated with the cleaning solution. A picture of the prototype wipe is in FIG. 31. In production the wipe can generally be of an optimum size and shape for use by the user.

The operation of the wipe is illustrated schematically in FIG. 30. Gentle pressure on the external surfaces of the wipe causes the water bubble 24 to burst so that the water comes into contact with the inorganic salt mixture 25 so that cooling is released. The temperature profile is illustrated graphically in FIG. 32.

### EXAMPLE 52

### Prototype Finger Shaped Heating Wipe

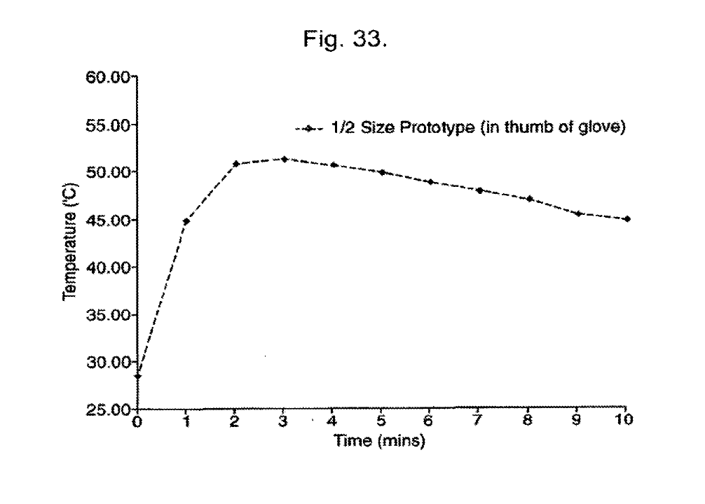

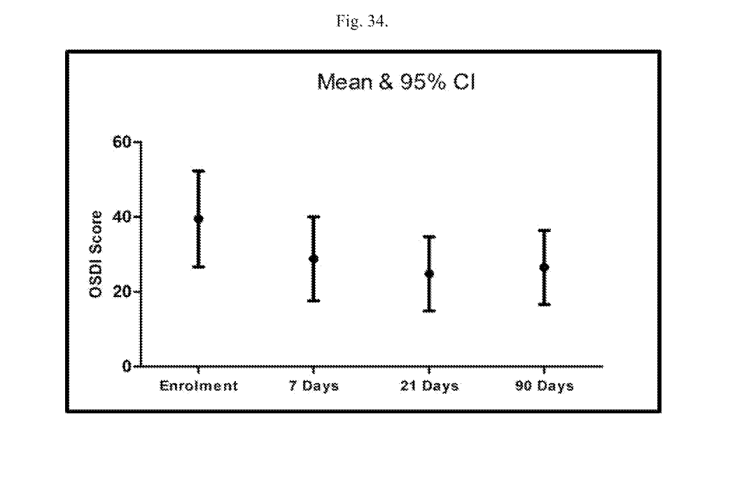

Example 35 was repeated except that the water bubbles and the salt mixture in the sealable water-tight pouch were placed in the thumb of a glove. The temperature profile achieved is illustrated graphically in FIG. 33.

### EXAMPLE 53

### Alternative Prototype

Two plastics sheets comprising three layers, an outer layer of polypropylene, an amorphous intermediate metallic oxide layer and an inner layer of polyethylene, are thermo-sealed around three sides to form a pouch. The pouch is then further sealed with a rupturable seal in the middle to create two chambers. A salt/PEG mixture is placed in one chamber and water into the other. The mouth of the pouch is then thermo-sealed. One external face of the pouch is coated with a polystyrene insulating layer and gauze is applied to the other face which when the wipe is in use can be in contact with the eyelid margin.

### EXAMPLE 54

### Effects of Controlled Heat Delivery to the Eyelid Margin & Eyelid Hygiene on Symptomatology and the Tear Film in Meibomian Gland Dysfunction (MGD)

This study assessed the efficacy of the combined use of controlled heat and eyelid massage. Two sequential devices were used: EyeGiene® (to produce controlled heat to the eyelid margins) and Supranettes™ eye cleaning wipes (to deliver eyelid massage): twice a day for three weeks and once a day for the reminder of the three month management. The population was eleven dry eye sufferers with MGD defined by a positive OSDI score (Total score≧13) and at least Grade 2 eyelid margin anomalies. The primary endpoints were subjective response with the OSDI, tear film stability measured with the Tearscope and tear film lipid composition measured by HPLC.

The study demonstrated the efficacy of the combined use of controlled heat and mechanical massage in reducing symptoms in MGD. Further, the results suggest that this controlled management affect the meibomian glands as the tear film lipid concentration increased, and its composition was modified leading to improved tear film stability.

Population
18 years or more
No Vision Correction OR Spectacle Wearer OR Soft (hydrogel or silicone hydrogel) Contact Lens Wearer
Dry Eye Symptomatology
   OSDI Score≧13
Mild or Moderate MGD
   Score≧2 on 0 to 4 Point Scales (On At Least One Parameter)
N=11 (9 non-wearers & 2 CL wearers)

Study Sesign

This study is a bilateral, prospective, interventional, open label investigation of three month duration including the following steps:

1. Enrolment

2. Dispensing visit

3. EyeGiene & Supranettes Twice daily

4. Follow-up visit after 7 & 21 days

5. EyeGiene & Supranettes Once daily

6. Follow-up visit after 90 days

The primary endpoints were measured changes in the tear film characteristics. The data was obtained by post hoc masked analysis: the technicians were masked as to the subject identity or time of collection. The secondary endpoints were subjective responses.

Results

The results showed that:

i. The average OSDI score decreased from “severe” pre-treatment (Total=39.5) to “moderate” at the three week (Total=24.8; p=0.031) and three month (Total=26.6; p=0.038) follow-up. At three months the incidence of severe cases was halved (73% vs. 36%) and the incidence of “asymptomatic” cases nearly one in five (0% vs. 18%).

ii. Tear film stability increased significantly between pre-treatment (Med 7.7 sec) and the three week (Med 12.7 sec; p=0.045) and three month (Med 11.3; p=0.004) follow-up.

iii. Total tear film lipid concentration increased significantly from pre-treatment (3.3 μl/μl) to the three week (5.8 μg/μl; p=0.079) and three month (7.4 μg/μl; p=0.032) follow-up.

iv. Tear film lipid composition was altered during the study period. After three months concentrations of fatty acids (12.6% vs. 7.2%; p=0.033) and cholesterol (1.4% vs. 0.9%; p=0.038) decreased and concentrations of wax ester/cholesterol ester (85.4% vs. 90.6%; p=0.039) and triglycerides (0.5% vs. 1.3%; p=0.013) increased.

Subjective Acceptance

1) OSDI Questionnaire

Twelve question validated questionnaire was used: Q1 to 5—Ocular and Vision related symptoms; Q6 to 9—Vision Questions; and Q10 to 12—Environmental Questions.

Scoring and classification system was based upon overall score:

0-12=Normal

13-22=Mild Dry Eye

23-32=Moderate Dry Eye

33-100=Severe Dry Eye

FIG. 34 shows mean score decreases in severity of dry eye symptoms in patients during the course of the study:

| | | |
|---|---|---|
| Day 7 - 10 pts | Day 21 - 15 pts | Day 90 - 13 pts |

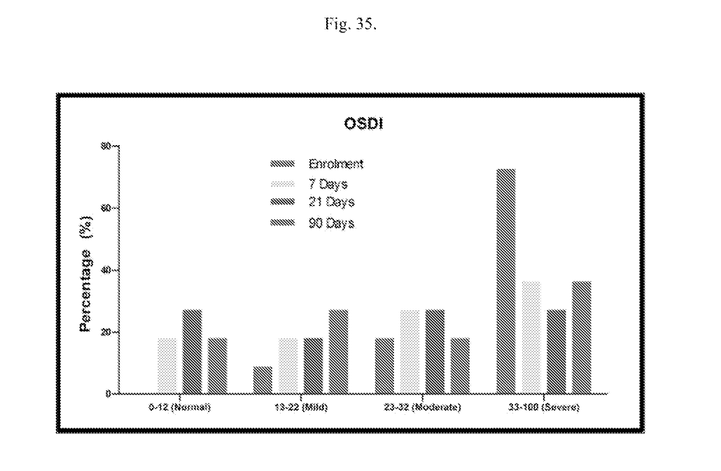

FIG. 35 shows percentage changes in normal, mild, moderate and severe dry eye incidences during the course of the study. Among the results, the percentages for severe cases are:

| | | | |
|---|---|---|---|
| Day 0 - 73% | Day 7 - 36% | Day 21 - 27% | Day 90 - 36% |

The percentages for asymptomatic (normal) cases are

| | | | |
|---|---|---|---|
| Day 0 - 0% | Day 7 - 18% | Day 21 - 27% | Day 90 - 18% |

2) Subjective Comfort

The subjective comfort was graded on a 100 point VAS scale with 0 Very poor to 100 Excellent in four rating areas:

Overall Comfort

Comfort Upon Waking=First half hour

Comfort Daytime=Up to 6:00 pm)

Comfort in the Evening=From 6:00 pm to sleep

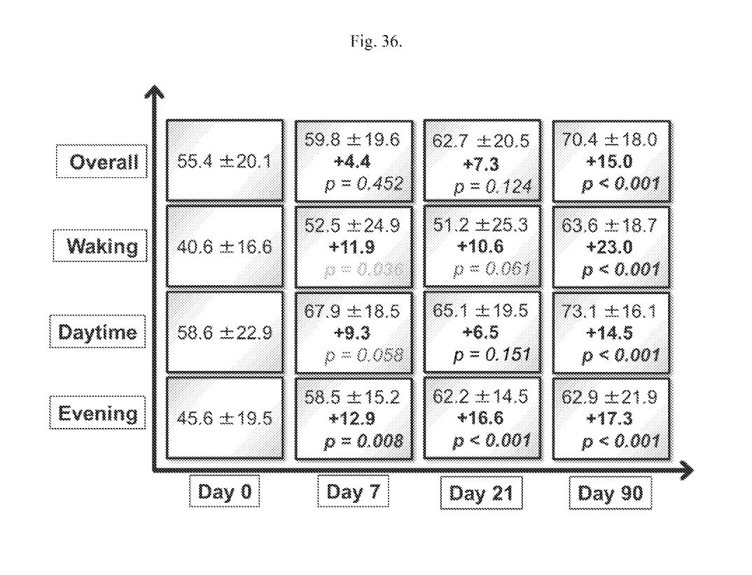

FIG. 36 shows comfort scores from patients during the course of the study. The results demonstrate a highly clinically significant increase in comfort for all times of day.

Tear Film Characteristics

Non Invasive Break Up Time (NIBUT)

Data capture was done by Keeler Tearscope: Topcon SL7 Slit-lamp & DV3 Digital Video Capture; data analysis was done with Tearscope Digital Video Post Hoc Masked Measurement.

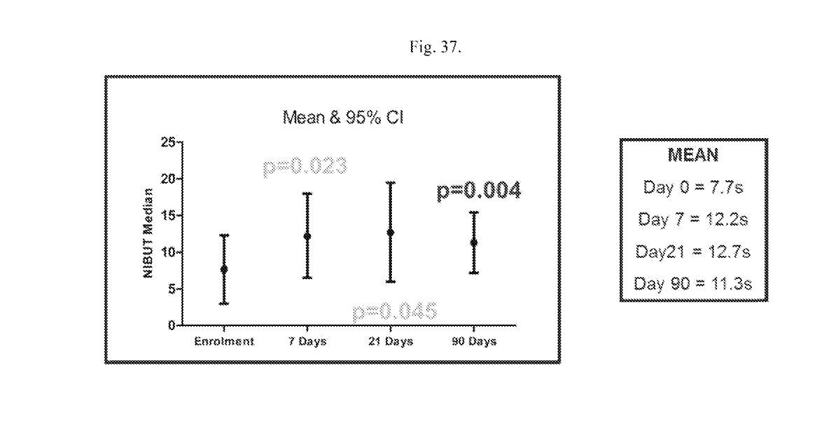

FIG. 37 shows mean increases in tear film stability in patients during the course of the study: Day 7—58%; Day 21—67%; Day 90—47%.

Tear Lipid Characteristics

HPLC analysis: TEAR SAMPLING with 1-5 μl Disposable Micropipettes; five main lipid classes were separated from the tear fluid:
1) Wax esters/Cholesterol esters (WE/CE)
2) Phospholipids
3) Triglycerides (TG)/Diglycerides/Monoglycerides
4) Fatty acids (FA)
5) Cholesterol (“CH” or “CHO”)

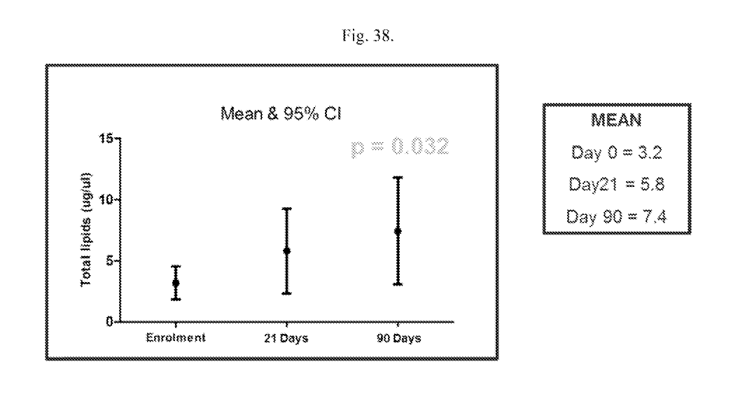

Total tear lipid concentration (μg/μl) is illustrated in FIG. 38. The results show a large increase in tear lipid concentration during the study period: Day 21=+81%; Day 90=+131%.

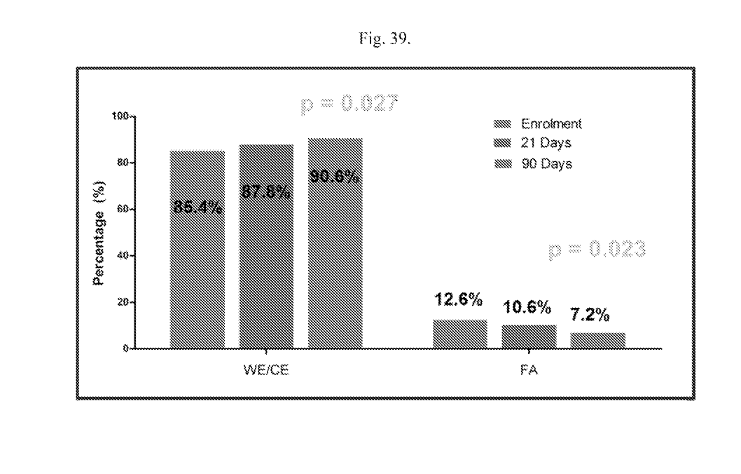

Tear concentration of WE/CE & FA (%) is illustrated in FIG. 39. The results show an increase in tear lipid WE/CE concentration and a decrease in tear lipid FA concentration.

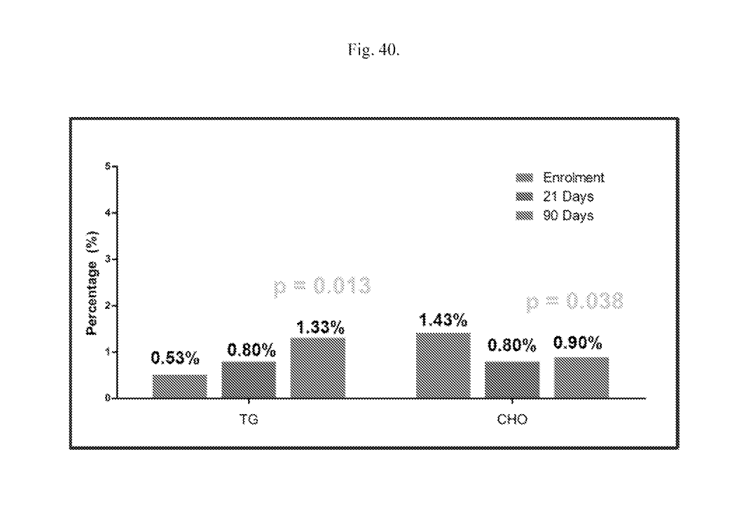

Tear concentration of TG & CH (%) is illustrated in FIG. 40. The results show an increase in tear lipid TG concentration and a decrease in tear lipid CH concentration.

What is claimed is:

1. A method of treating an eyelid disorder in a subject, comprising applying a wipe to an eye region of the subject, wherein the wipe generates heat and is adapted for massage, and wherein the application of the wipe increases stability of a tear film of the subject, modulates a lipid concentration in a tear film of the subject, and/or modulates a lipid proportion in a total lipid composition of a tear film of the subject.

2. A method of increasing stability of a tear film of a subject, modulating a lipid concentration in a tear film of a subject, and/or modulating a lipid proportion in a total lipid composition of the tear film of a subject, comprising applying a wipe to an eye region of the subject, wherein the wipe generates heat and is adapted for massage, and wherein the subject suffers from an eyelid disorder.

3. The method of claim 1 or 2, wherein the eyelid disorder is blepharitis, dryness, irritation, redness, inflammation, meibomian gland dysfunction, hay fever, a condition associated with use of eye cosmetics, or a condition associated with contact-lens wear.

4. The method of any preceding claim, wherein the eyelid disorder is meibomian gland dysfunction.

5. The method of any preceding claim, wherein a non-invasive break-up time (NIBUT) of a tear film of the subject is increased after the application of the wipe in comparison to a NIBUT of a tear film of the subject before the application of the wipe.

6. The method of claim 5, wherein the NIBUT of the tear film of the subject is increased after the application of the wipe by about 30%-100% in comparison to the NIBUT of the tear film of the subject before the application of the wipe.

7. The method of claim 6, wherein the NIBUT of the tear film of the subject is increased after the application of the wipe by about: 30%-40%, 40%-50%, 50%-60%, 60%-70%, 70%-80%, 80%-90%, or 90%-100% in comparison to the NIBUT of the tear film of the subject before the application of the wipe.

8. The method of claim 6, wherein the NIBUT of the tear film of the subject is increased after the application of the wipe by about: 30%-90%, 40%-80%, 50%-70%, or 55%-65% in comparison to the NIBUT of the tear film of the subject before the application of the wipe.

9. The method of claim 6, wherein the NIBUT of the tear film of the subject is increased after the application of the wipe by about: 30%, 35%, 40%, 45%, 50%, 55%, 60%, 65%, 70%, 75%, 80%, 85%, 90%, 95%, or 100% in comparison to the NIBUT of the tear film of the subject before the application of the wipe.

10. The method of claim 6, wherein the NIBUT of the tear film of the subject is increased by about 55%-65% in about 1-14 days after beginning the application of the wipe in comparison to the NIBUT of the tear film of the subject before the application of the wipe.

11. The method of claim 6, wherein the NIBUT of the tear film of the subject is increased by about 58% in about 7 days after beginning the application of the wipe in comparison to the NIBUT of the tear film of the subject before the application of the wipe.

12. The method of any one of claims 5-11, wherein the NIBUT of the tear film of the subject is increased by about 60%-70% in about 1-42 days after beginning the application of the wipe in comparison to the NIBUT of the tear film of the subject before the application of the wipe.

13. The method of claim 12, wherein the NIBUT of the tear film of the subject is increased by about 65% in about 21 days after beginning the application of the wipe in comparison to the NIBUT of the tear film of the subject before the application of the wipe.

14. The method of any one of claims 5-13, wherein the NIBUT of the tear film of the subject has an increase of about 1-10 seconds after the application of the wipe in comparison to the NIBUT of the tear film of the subject before the application of the wipe.

15. The method of claim 14, wherein the NIBUT of the tear film of the subject has an increase of about: 1-2, 2-3, 3-4, 4-5, 5-6, 6-7, 7-8, 8-9, or 9-10 seconds after the application of the wipe in comparison to the NIBUT of the tear film of the subject before the application of the wipe.

16. The method of claim 14, wherein the NIBUT of the tear film of the subject has an increase of about: 1-9, 2-8, 3-7, 4-6, or 4.5-5.5 seconds after the application of the wipe in comparison to the NIBUT of the tear film of the subject before the application of the wipe.

17. The method of claim 14, wherein the NIBUT of the tear film of the subject has an increase of about: 1, 1.5, 2, 2.5, 3, 3.5, 4, 4.5, 5, 5.5, 6, 6.5, 7, 7.5, 8, 8.5, 9, 9.5, or 10 seconds after the application of the wipe in comparison to the NIBUT of the tear film of the subject before the application of the wipe.

18. The method of claim 14, wherein the NIBUT of the tear film of the subject has an increase of about 4-5 seconds in about 1-14 days after beginning the application of the wipe in comparison to the NIBUT of the tear film of the subject before the application of the wipe.

19. The method of claim 14, wherein the NIBUT of the tear film of the subject has an increase of about 4.5 seconds in about 7 days after beginning the application of the wipe in comparison to the NIBUT of the tear film of the subject before the application of the wipe.

20. The method of any one of claims 14-19, wherein the NIBUT of the tear film of the subject has an increase of about 4-6 seconds in about 1-42 days after beginning the application of the wipe in comparison to the NIBUT of the tear film of the subject before the application of the wipe.

21. The method of claim 20, wherein the NIBUT of the tear film of the subject has an increase of about 5 seconds in about 21 days after beginning the application of the wipe in comparison to the NIBUT of the tear film of the subject before the application of the wipe.

22. The method of any preceding claim, wherein a total lipid concentration in a tear film of the subject is increased after the application of the wipe in comparison to a total lipid concentration of a tear film of the subject before the application of the wipe.

23. The method of claim 22, wherein the total lipid concentration in the tear film of the subject is increased after the application of the wipe by about 50%-200% in comparison to the total lipid concentration of the tear film of the subject before the application of the wipe.

24. The method of claim 23, wherein the total lipid concentration in the tear film of the subject is increased after the application of the wipe by about: 50%-60%, 60%-70%, 70%-80%, 80%-90%, 90%-100%, 100%-110%, 110%-120%, 120%-130%, 130%-140%, 140%-150%, 150%-160%, 160-170%, 170%-180%, 180%-190%, or 190%-200% in comparison to the total lipid concentration of the tear film of the subject before the application of the wipe.

25. The method of claim 23, wherein the total lipid concentration in the tear film of the subject is increased after the application of the wipe by about: 50%-100%, 60%-110%, 70%-90%, 100%-160%, 110%-150%, or 120%-140% in comparison to the total lipid concentration of the tear film of the subject before the application of the wipe.

26. The method of claim 23, wherein the total lipid concentration in the tear film of the subject is increased after the application of the wipe by about: 50%, 60%, 70%, 80%, 90%, 100%, 110%, 120%, 130%, 140%, 150%, 160%, 170%, 180%, 190%, or 200% in comparison to the total lipid concentration of the tear film of the subject before the application of the wipe.

27. The method of claim 23, wherein the total lipid concentration in the tear film of the subject is increased by about 75-85% in about 1-42 days after beginning the application of the wipe in comparison to the total lipid concentration of the tear film of the subject before the application of the wipe.

28. The method of claim 23, wherein the total lipid concentration in the tear film of the subject is increased by about 81% in about 21 days after beginning the application of the wipe in comparison to the total lipid concentration of the tear film of the subject before the application of the wipe.

29. The method of any one of claims 22-28, wherein the total lipid concentration in the tear film of the subject is increased by about 120-140% in about 60-120 days after beginning the application of the wipe in comparison to the total lipid concentration of the tear film of the subject before the application of the wipe.

30. The method of claim 29, wherein the total lipid concentration in the tear film of the subject is increased by about 131% in about 90 days after beginning the application of the wipe in comparison to the total lipid concentration of the tear film of the subject before the application of the wipe.

31. The method of any one of claims 22-30, wherein the total lipid concentration in the tear film of the subject has an increase of about 1-10 μg/μl after the application of the wipe in comparison to the total lipid concentration of the tear film of the subject before the application of the wipe.

32. The method of claim 31, wherein the total lipid concentration in the tear film of the subject has an increase of about: 1-2 μg/μl, 2-3 μg/μl, 3-4 μg/μl, 4-5 μg/μl, 5-6 μg/μl, 6-7 μg/μl, 7-8 μg/μl, 8-9 μg/μl, or 9-10 μg/μl after the application of the wipe in comparison to the total lipid concentration of the tear film of the subject before the application of the wipe.

33. The method of claim 31, wherein the total lipid concentration in the tear film of the subject has an increase of about: 1-6 μg/μl, 2-5 μg/μl, 2-3.5 μg/μl, or 3.5-5 μg/μl after the application of the wipe in comparison to the total lipid concentration of the tear film of the subject before the application of the wipe.

34. The method of claim 31, wherein the total lipid concentration in the tear film of the subject has an increase of about: 1, 1.5, 2, 2.5, 3, 3.5, 4, 4.5, 5, 5.5, 6, 6.5, 7, 7.5, 8, 8.5, 9, 9.5, or 10 μg/μl after the application of the wipe in comparison to the total lipid concentration of the tear film of the subject before the application of the wipe.

35. The method of claim 31, wherein the total lipid concentration in the tear film of the subject has an increase of about 2-3 μg/μl in about 1-42 days after beginning the application of the wipe in comparison to the total lipid concentration of the tear film of the subject before the application of the wipe.

36. The method of claim 31, wherein the total lipid concentration in the tear film of the subject has an increase of about 2.6 μg/μl in about 21 days after beginning the application of the wipe in comparison to the total lipid concentration of the tear film of the subject before the application of the wipe.

37. The method of any one of claims 31-36, wherein the total lipid concentration in the tear film of the subject has an increase of about 4-5 μg/μl in about 60-120 days after beginning the application of the wipe in comparison to the total lipid concentration of the tear film of the subject before the application of the wipe.

38. The method of claim 37, wherein the total lipid concentration in the tear film of the subject has an increase of about 4.2 μg/μl in about 90 days after beginning the application of the wipe in comparison to the total lipid concentration of the tear film of the subject before the application of the wipe.

39. The method of any preceding claim, wherein a proportion of wax ester or cholesterol ester in a total lipid composition of a tear film of the subject is increased after the application of the wipe in comparison to a proportion of wax ester or cholesterol ester in a total lipid composition of a tear film of the subject before the application of the wipe.

40. The method of claim 39, wherein the proportion of wax ester or cholesterol ester in the total lipid composition of the tear film of the subject is increased after the application of the wipe by about 1%-20% in comparison to the proportion of wax ester or cholesterol ester in the total lipid composition of the tear film of the subject before the application of the wipe.

41. The method of claim 40, wherein the proportion of wax ester or cholesterol ester in the total lipid composition of the tear film of the subject is increased after the application of the wipe by about: 1%-2%, 2%-4%, 4%-6%, 6%-8%, 8%-10%, 10%-12%, 12%-14%, 14%-16%, 16%-18%, or 18%-20% in comparison to the proportion of wax ester or cholesterol ester in the total lipid composition of the tear film of the subject before the application of the wipe.

42. The method of claim 40, wherein the proportion of wax ester or cholesterol ester in the total lipid composition of the tear film of the subject is increased after the application of the wipe by about: 1%-8%, 2%-7%, 3%-6%, 2%-4%, 5%-7%, 4%-8%, or 1%-5% in comparison to the proportion of wax ester or cholesterol ester in the total lipid composition of the tear film of the subject before the application of the wipe.

43. The method of claim 40, wherein the proportion of wax ester or cholesterol ester in the total lipid composition of the tear film of the subject is increased after the application of the wipe by about: 1%, 2%, 3%, 4%, 5%, 6%, 7%, 8%, 9%, 10%, 11%, 12%, 13%, 14%, 15%, 16%, 17%, 18%, 19%, or 20% in comparison to the proportion of wax ester or cholesterol ester in the total lipid composition of the tear film of the subject before the application of the wipe.

44. The method of claim 40, wherein the proportion of wax ester or cholesterol ester in the total lipid composition of the tear film of the subject is increased by about 2-4% in about 1-42 days after beginning the application of the wipe in comparison to the proportion of wax ester or cholesterol ester in the total lipid composition of the tear film of the subject before the application of the wipe.

45. The method of claim 40, wherein the proportion of wax ester or cholesterol ester in the total lipid composition of the tear film of the subject is increased by about 3% in about 21 days after beginning the application of the wipe in comparison to the proportion of wax ester or cholesterol ester in the total lipid composition of the tear film of the subject before the application of the wipe.

46. The method of any one of claims 40-45, wherein the proportion of wax ester or cholesterol ester in the total lipid composition of the tear film of the subject is increased by about 5-7% in about 60-120 days after beginning the application of the wipe in comparison to the proportion of wax ester or cholesterol ester in the total lipid composition of the tear film of the subject before the application of the wipe.

47. The method of claim 46, wherein the proportion of wax ester or cholesterol ester in the total lipid composition of the tear film of the subject is increased by about 6% in about 90 days after beginning the application of the wipe in comparison to the proportion of wax ester or cholesterol ester in the total lipid composition of the tear film of the subject before the application of the wipe.

48. The method of any one of claims 39-47, wherein a proportion of wax ester and cholesterol ester in the total lipid composition of the tear film of the subject has an increase of about 1%-10% after the application of the wipe in comparison to a proportion of wax ester and cholesterol ester in the total lipid composition of the tear film of the subject before the application of the wipe.

49. The method of claim 48, wherein the proportion of wax ester and cholesterol ester in the total lipid composition of the tear film of the subject has an increase of about: 1%-2%, 2%-3%, 3%-4%, 4%-5%, 5%-6%, 6%-7%, 7%-8%, 8%-9%, or 9%-10% after the application of the wipe in comparison to the proportion of wax ester and cholesterol ester in the total lipid composition of the tear film of the subject before the application of the wipe.

50. The method of claim 48, wherein the proportion of wax ester and cholesterol ester in the total lipid composition of the tear film of the subject has an increase of about: 1%-7%, 2-6%, 1.5%-3.5%, or 4.5%-6.5% after the application of the wipe in comparison to the proportion of wax ester and cholesterol ester in the total lipid composition of the tear film of the subject before the application of the wipe.

51. The method of claim 48, wherein the proportion of wax ester and cholesterol ester in the total lipid composition of the tear film of the subject has an increase of about: 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10% after the application of the wipe in comparison to the proportion of wax ester and cholesterol ester in the total lipid composition of the tear film of the subject before the application of the wipe.

52. The method of claim 48, wherein the proportion of wax ester and cholesterol ester in the total lipid composition of the tear film of the subject has an increase of about 2-3% in about 1-42 days after beginning the application of the wipe in comparison to the proportion of wax ester and cholesterol ester in the total lipid composition of the tear film of the subject before the application of the wipe.

53. The method of claim 48, wherein the proportion of wax ester and cholesterol ester in the total lipid composition of the tear film of the subject has an increase of about 2.4% in about 21 days after beginning the application of the wipe in comparison to the proportion of wax ester and cholesterol ester in the total lipid composition of the tear film of the subject before the application of the wipe.

54. The method of any one of claims 48-53, wherein the proportion of wax ester and cholesterol ester in the total lipid composition of the tear film of the subject has an increase of about 4-6% in about 60-120 days after beginning the application of the wipe in comparison to the proportion of wax ester and cholesterol ester in the total lipid composition of the tear film of the subject before the application of the wipe.

55. The method of claim 54, wherein the proportion of wax ester and cholesterol ester in the total lipid composition of the tear film of the subject has an increase of about 5.2% in about 90 days after beginning the application of the wipe in comparison to the proportion of wax ester and cholesterol ester in the total lipid composition of the tear film of the subject before the application of the wipe.

56. The method of any preceding claim, wherein a proportion of fatty acids in a total lipid composition of a tear film of the subject is decreased after the application of the wipe in comparison to a proportion of fatty acids in the total lipid composition of a tear film of the subject before the application of the wipe.

57. The method of claim 56, wherein the proportion of fatty acids in the total lipid composition of the tear film of the subject is decreased after the application of the wipe by about 5%-80% in comparison to the proportion of fatty acids in the total lipid composition of the tear film of the subject before the application of the wipe.

58. The method of claim 57, wherein the proportion of fatty acids in the total lipid composition of the tear film of the subject is decreased after the application of the wipe by about 5%-10%, 10%-15%, 15%-20%, 20%-25%, 25%-30%, 30%-35%, 35%-40%, 40%-45%, 45%-50%, 50%-55%, 55%-60%, 65%-70%, 70%-75%, or 75%-80% in comparison to the proportion of fatty acids in the total lipid composition of the tear film of the subject before the application of the wipe.

59. The method of claim 57, wherein the proportion of fatty acids in the total lipid composition of the tear film of the subject is decreased after the application of the wipe by about 5%-60%, 10%-50%, 5%-25%, 5%-30%, 10%-20%, 40%-45%, 30%-50%, 30%-60%, 20%-60%, or 10%-70% in comparison to the proportion of fatty acids in the total lipid composition of the tear film of the subject before the application of the wipe.

60. The method of claim 57, wherein the proportion of fatty acids in the total lipid composition of the tear film of the subject is decreased after the application of the wipe by about: 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, or 80% in comparison to the proportion of fatty acids in the total lipid composition of the tear film of the subject before the application of the wipe.

61. The method of claim 57, wherein the proportion of fatty acids in the total lipid composition of the tear film of the subject is decreased by about 5-30% in about 1-42 days after beginning the application of the wipe in comparison to the proportion of fatty acids in the total lipid composition of the tear film of the subject before the application of the wipe.

62. The method of claim 57, wherein the proportion of fatty acids in the total lipid composition of the tear film of the subject is decreased by about 16% in about 21 days after beginning the application of the wipe in comparison to the proportion of fatty acids in the total lipid composition of the tear film of the subject before the application of the wipe.

63. The method of any one of claims 57-62, wherein the proportion of fatty acids in the total lipid composition of the tear film of the subject is decreased by about 30-60% in about 60-120 days after beginning the application of the wipe in comparison to the proportion of fatty acids in the total lipid composition of the tear film of the subject before the application of the wipe.

64. The method of claim 63, wherein the proportion of fatty acids in the total lipid composition of the tear film of the subject is decreased by about 43% in about 90 days after beginning the application of the wipe in comparison to the proportion of fatty acids in the total lipid composition of the tear film of the subject before the application of the wipe.

65. The method of any one of claims 56-64, wherein the proportion of fatty acids in the total lipid composition of the tear film of the subject has a decrease of about 1%-10% after the application of the wipe in comparison to the proportion of fatty acids in the total lipid composition of the tear film of the subject before the application of the wipe.

66. The method of claim 65, wherein the proportion of fatty acids in the total lipid composition of the tear film of the subject has a decrease of about: 1%-2%, 2%-3%, 3%-4%, 4%-5%, 5%-6%, 6%-7%, 7%-8%, 8%-9%, or 9%-10% after the application of the wipe in comparison to the proportion of fatty acids in the total lipid composition of the tear film of the subject before the application of the wipe.

67. The method of claim 65, wherein the proportion of fatty acids in the total lipid composition of the tear film of the subject has a decrease of about: 1%-7%, 1%-6%, 1%-3%, 4%-6%, or 4%-7% after the application of the wipe in comparison to the proportion of fatty acids in the total lipid composition of the tear film of the subject before the application of the wipe.

68. The method of claim 65, wherein the proportion of fatty acids in the total lipid composition of the tear film of the subject has a decrease of about: 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10% after the application of the wipe in comparison to the proportion of fatty acids in the total lipid composition of the tear film of the subject before the application of the wipe.

69. The method of claim 65, wherein the proportion of fatty acids in the total lipid composition of the tear film of the subject has a decrease of about 1-3% in about 1-42 days after beginning the application of the wipe in comparison to the proportion of fatty acids in the total lipid composition of the tear film of the subject before the application of the wipe.

70. The method of claim 65, wherein the proportion of fatty acids in the total lipid composition of the tear film of the subject has a decrease of about 2% in about 21 days after beginning the application of the wipe in comparison to the proportion of fatty acids in the total lipid composition of the tear film of the subject before the application of the wipe.

71. The method of any one of claims 65-70, wherein the proportion of fatty acids in the total lipid composition of the tear film of the subject has a decrease of about 4-7% in about 60-120 days after beginning the application of the wipe in comparison to the proportion of fatty acids in the total lipid composition of the tear film of the subject before the application of the wipe.

72. The method of claim 71, wherein the proportion of fatty acids in the total lipid composition of the tear film of the subject has a decrease of about 5.4% in about 90 days after beginning the application of the wipe in comparison to the proportion of fatty acids in the total lipid composition of the tear film of the subject before the application of the wipe.

73. The method of any preceding claim, wherein a proportion of triglycerides in a total lipid composition of a tear film of the subject is increased after the application of the wipe in comparison to a proportion of triglycerides in a total lipid composition of a tear film of the subject before the application of the wipe.

74. The method of claim 73, wherein the proportion of triglycerides in the total lipid composition of the tear film of the subject is increased after the application of the wipe by about 30%-200% in comparison to the proportion of triglycerides in the total lipid composition of the tear film of the subject before the application of the wipe.

75. The method of claim 74, wherein the proportion of triglycerides in the total lipid composition of the tear film of the subject is increased after the application of the wipe by about: 30%-40%, 40%-50%, 50%-60%, 60%-70%, 70%-80%, 80%-90%, 90%-100%, 100%-110%, 110%-120%, 120%-130%, 130%-140%, 140%-150%, 150%-160%, 160-170%, 170%-180%, 180%-190%, or 190%-200% in comparison to the proportion of triglycerides in the total lipid composition of the tear film of the subject before the application of the wipe.

76. The method of claim 74, wherein the proportion of triglycerides in the total lipid composition of the tear film of the subject is increased after the application of the wipe by about: 30%-70%, 40%-60%, 130%-190%, 140%-180%, 30%-180%, 40%-170%, or 50%-160% in comparison to the proportion of triglycerides in the total lipid composition of the tear film of the subject before the application of the wipe.

77. The method of claim 74, wherein the proportion of triglycerides in the total lipid composition of the tear film of the subject is increased after the application of the wipe by about: 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, or 200% in comparison to the proportion of triglycerides in the total lipid composition of the tear film of the subject before the application of the wipe.

78. The method of claim 74, wherein the proportion of triglycerides in the total lipid composition of the tear film of the subject is increased by about 40-60% in about 1-42 days after beginning the application of the wipe in comparison to the proportion of triglycerides in the total lipid composition of the tear film of the subject before the application of the wipe.

79. The method of claim 74, wherein the proportion of triglycerides in the total lipid composition of the tear film of the subject is increased by about 51% in about 21 days after beginning the application of the wipe in comparison to the proportion of triglycerides in the total lipid composition of the tear film of the subject before the application of the wipe.

80. The method of any one of claims 74-79, wherein the proportion of triglycerides in the total lipid composition of the tear film of the subject is increased by about 120%-180% in about 60-120 days after beginning the application of the wipe in comparison to the proportion of triglycerides in the total lipid composition of the tear film of the subject before the application of the wipe.

81. The method of claim 80, wherein the proportion of triglycerides in the total lipid composition of the tear film of the subject is increased by about 151% in about 90 days after beginning the application of the wipe in comparison to the proportion of triglycerides in the total lipid composition of the tear film of the subject before the application of the wipe.

82. The method of any one of claims 73-81, wherein the proportion of triglycerides in the total lipid composition of the tear film of the subject has an increase of about 0.1%-3% after the application of the wipe in comparison to the proportion of triglycerides in the total lipid composition of the tear film of the subject before the application of the wipe.

83. The method of claim 82, wherein the proportion of triglycerides in the total lipid composition of the tear film of the subject has an increase of about: 0.1%-1%, 0.2%-0.9%, 0.2%-0.4%, 0.25%-0.35%, 0.75%-0.85%, 0.7%-0.9%, 0.6%-1.0%, or 0.1%-1.5% after beginning the application of the wipe in comparison to the proportion of triglycerides in the total lipid composition of the tear film of the subject before the application of the wipe.

84. The method of claim 82, wherein the proportion of triglycerides in the total lipid composition of the tear film of the subject has an increase of about: 0.1%-0.2%, 0.2%-0.4%, 0.4%-0.6%, 0.6%-0.8%, 0.8%-1.0%, 1%-1.2%, 1.2%-1.4%, 1.4%-1.6%, 1.6%-1.8%, 1.8%-2%, 2%-2.2%, 2.2%-2.4%, 2.4%-2.6%, 2.6%-2.8%, or 2.8%-3% after beginning the application of the wipe in comparison to the proportion of triglycerides in the total lipid composition of the tear film of the subject before the application of the wipe.

85. The method of claim 82, wherein the proportion of triglycerides in the total lipid composition of the tear film of the subject has an increase of about: 0.1%, 0.15%, 0.2%, 0.25%, 0.3%, 0.35%, 0.4%, 0.45%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.5%, or 3% after the application of the wipe in comparison to the proportion of triglycerides in the total lipid composition of the tear film of the subject before the application of the wipe.

86. The method of claim 82, wherein the proportion of triglycerides in the total lipid composition of the tear film of the subject has an increase of about 0.2-0.4% in about 1-42 days after beginning the application of the wipe in comparison to the proportion of triglycerides in the total lipid composition of the tear film of the subject before the application of the wipe.

87. The method of claim 82, wherein the proportion of triglycerides in the total lipid composition of the tear film of the subject has an increase of about 0.3% in about 21 days after beginning the application of the wipe in comparison to the proportion of triglycerides in the total lipid composition of the tear film of the subject before the application of the wipe.

88. The method of any one of claims 82-87, wherein the proportion of triglycerides in the total lipid composition of the tear film of the subject has an increase of about 0.6-1.0% in about 60-120 days after beginning the application of the wipe in comparison to the proportion of triglycerides in the total lipid composition of the tear film of the subject before the application of the wipe.

89. The method of claim 88, wherein the proportion of triglycerides in the total lipid composition of the tear film of the subject has an increase of about 0.8% in about 90 days after beginning the application of the wipe in comparison to the proportion of triglycerides in the total lipid composition of the tear film of the subject before the application of the wipe.

90. The method of any preceding claim, wherein a proportion of cholesterol in a total lipid composition of a tear film of the subject is decreased after the application of the wipe in comparison to a proportion of cholesterol in a total lipid composition of a tear film of the subject before the application of the wipe.

91. The method of claim 90, wherein the proportion of cholesterol in the total lipid composition of the tear film of the subject is decreased after the application of the wipe by about 10%-80% in comparison to the proportion of cholesterol in the total lipid composition of the tear film of the subject before the application of the wipe.

92. The method of claim 91, wherein the proportion of cholesterol in the total lipid composition of the tear film of the subject is decreased after the application of the wipe by about: 10%-15%, 15%-20%, 20%-25%, 25%-30%, 30%-35%, 35%-40%, 40%-45%, 45%-50%, 50%-55%, 55%-60%, 65%-70%, 70%-75%, or 75%-80% in comparison to the proportion of cholesterol in the total lipid composition of the tear film of the subject before the application of the wipe.

93. The method of claim 91, wherein the proportion of cholesterol in the total lipid composition of the tear film of the subject is decreased after the application of the wipe by about: 10%-70%, 20%-60%, or 30%-50% in comparison to the proportion of cholesterol in the total lipid composition of the tear film of the subject before the application of the wipe.

94. The method of claim 91, wherein the proportion of cholesterol in the total lipid composition of the tear film of the subject is decreased after the application of the wipe by about: 10%, 15%, 20%, 25%, 30%, 35%, 40%, 45%, 50%, 55%, 60%, 65%, 70%, 75%, or 80% in comparison to the proportion of cholesterol in the total lipid composition of the tear film of the subject before the application of the wipe.

95. The method of claim 91, wherein the proportion of cholesterol in the total lipid composition of the tear film of the subject is decreased by about 35-55% in about 1-42 days after beginning the application of the wipe in comparison to the proportion of cholesterol in the total lipid composition of the tear film of the subject before the application of the wipe.

96. The method of claim 91, wherein the proportion of cholesterol in the total lipid composition of the tear film of the subject is decreased by about 44% in about 21 days after beginning the application of the wipe in comparison to the proportion of cholesterol in the total lipid composition of the tear film of the subject before the application of the wipe.

97. The method of any one of claims 91-96, wherein the proportion of cholesterol in the total lipid composition of the tear film of the subject is decreased by about 30-50% in about 60-120 days after beginning the application of the wipe in comparison to the proportion of cholesterol in the total lipid composition of the tear film of the subject before the application of the wipe.

98. The method of claim 97, wherein the proportion of cholesterol in the total lipid composition of the tear film of the subject is decreased by about 37% in about 90 days after beginning the application of the wipe in comparison to the proportion of cholesterol in the total lipid composition of the tear film of the subject before the application of the wipe.

99. The method of any one of claims 90-98, wherein the proportion of cholesterol in the total lipid composition of the tear film of the subject has a decrease of about 0.1-3% after the application of the wipe in comparison to the proportion of cholesterol in the total lipid composition of the tear film of the subject before the application of the wipe.

100. The method of claim 99, wherein the proportion of cholesterol in the total lipid composition of the tear film of the subject has a decrease of about: 0.1%-0.2%, 0.2%-0.4%, 0.4%-0.6%, 0.6%-0.8%, 0.8%-1.0%, 1%-1.2%, 1.2%-1.4%, 1.4%-1.6%, 1.6%-1.8%, 1.8%-2%, 2%-2.2%, 2.2%-2.4%, 2.4%-2.6%, 2.6%-2.8%, or 2.8%-3% after the application of the wipe in comparison to the proportion of cholesterol in the total lipid composition of the tear film of the subject before the application of the wipe.

101. The method of claim 99, wherein the proportion of cholesterol in the total lipid composition of the tear film of the subject has a decrease of about: 0.4%-0.6%, 0.5%-0.6%, 0.4%-0.7%, 0.5%-0.7%, 0.5%-0.8%, 0.3%-0.8%, or 0.3%-0.7% after the application of the wipe in comparison to the proportion of cholesterol in the total lipid composition of the tear film of the subject before the application of the wipe.

102. The method of claim 99, wherein the proportion of cholesterol in the total lipid composition of the tear film of the subject has a decrease of about: 0.1%, 0.15%, 0.2%, 0.25%, 0.3%, 0.35%, 0.4%, 0.45%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.5%, or 3% after the application of the wipe in comparison to the proportion of cholesterol in the total lipid composition of the tear film of the subject before the application of the wipe.

103. The method of claim 99, wherein the proportion of cholesterol in the total lipid composition of the tear film of the subject has a decrease of about 0.4-0.8% in about 1-42 days after beginning the application of the wipe in comparison to the proportion of cholesterol in the total lipid composition of the tear film of the subject before the application of the wipe.

104. The method of claim 99, wherein the proportion of cholesterol in the total lipid composition of the tear film of the subject has a decrease of about 0.63% in about 21 days after beginning the application of the wipe in comparison to the proportion of cholesterol in the total lipid composition of the tear film of the subject before the application of the wipe.

105. The method of any one of claims 99-104, wherein the proportion of cholesterol in the total lipid composition of the tear film of the subject has a decrease of about 0.4-0.7% in about 60-120 days after beginning the application of the wipe in comparison to the proportion of cholesterol in the total lipid composition of the tear film of the subject before the application of the wipe.

106. The method of claim 105, wherein the proportion of cholesterol in the total lipid composition of the tear film of the subject has a decrease of about 0.53% in about 90 days after beginning the application of the wipe in comparison to the proportion of cholesterol in the total lipid composition of the tear film of the subject before the application of the wipe.

107. The method of any preceding claim, wherein the ratio of wax ester or cholesterol ester; fatty acids; triglycerides; and cholesterol in the tear film of the subject is about (86 to 91):(13 to 7):(0.6 to 1.3):(1.5 to 0.8) after the application of the wipe.

108. The method of claim 107, wherein the ratio of wax ester or cholesterol ester; fatty acids; triglycerides; and cholesterol in the tear film of the subject is about (87.8):(10.6):(0.8):(0.8) in about 7-35 days after beginning the application of the wipe.

109. The method of claim 108, wherein the ratio of wax ester or cholesterol ester; fatty acids; triglycerides; and cholesterol in the tear film of the subject is about (87.8):(10.6):(0.8):(0.8) in about 21 days after beginning the application of the wipe.

110. The method of any preceding claim, wherein the ratio of wax ester or cholesterol ester; fatty acids; triglycerides; and cholesterol in the tear film of the subject is about (90.6):(7.2):(1.3):(0.9) in about 35-120 days after beginning the application of the wipe.

111. The method of claim 110, wherein the ratio of wax ester or cholesterol ester; fatty acids; triglycerides; and cholesterol in the tear film of the subject is about (90.6):(7.2):(1.3):(0.9) in about 90 days after beginning the application of the wipe.

112. The method of any preceding claim, wherein the massage is mechanical.

113. The method of any preceding claim, wherein the massage is operated by a hand.

114. The method of claim 113, wherein the massage is operated by a finger, palm, or back of the hand.

115. The method of any preceding claim, wherein the massage is conducted to the eye region of the subject with the wipe.

116. The method of any preceding claim, wherein the massage is conducted after the heat is generated.

117. The method of any preceding claim, further comprising holding the wipe on the eye region of the subject for a period of time.

118. The method of claim 117, wherein the period of time is from about 1 minute to about 15 minutes.

119. The method of claim 118, wherein the period of time is about: 1-2, 2-3, 3-4, 4-5, 5-6, 6-7, 7-8, 8-9, 9-10, 10-11, 11-12, 12-13, 13-14, or 14-15 minutes.

120. The method of claim 118, wherein the period of time is about: 1-5, 2-5, 3-5, 1-10, 2-8, or 4-6 minutes.

121. The method of claim 118, wherein the period of time is about: 1, 2, 3, 4, 5, 6, 7, 8, 9, 10, 11, 12, 13, 14, or 15 minute(s).

122. The method of any preceding claim, wherein the method is repeated one or more times every day or every 2 days.

123. The method of claim 122, wherein the method is repeated at least 2 times per day.

124. The method of claim 122, wherein the method is repeated 1, 2, 3, 4, or 5 times per day.

125. The method of claim 122, wherein the method is repeated once or twice a day.

126. The method of any preceding claim, wherein the method is repeated for about 1-120 days.

127. The method of claim 126, wherein the method is repeated for about: 1-3, 3-7, 7-14, 14-21, 21-28, 28-35, 35-42, 42-49, 49-56, 56-63, 63-70, 70-80, 80-90, 90-100, 100-110, or 110-120 days.

128. The method of claim 126, wherein the method is repeated for about: 1-7, 1-14, 1-21, 1-28, 1-35, 1-42, 1-49, 1-56, 1-63, 1-70, 1-80, 1-90, 1-100, or 1-110 days.

129. The method of claim 126, wherein the method is repeated for about: 3, 7, 14, 21, 28, 35, 42, 49, 56, 63, 70, 80, 90, 100, 110, or 120 days.

130. The method of any preceding claim, wherein the method is repeated 1-4 times per day for 1-5 weeks and then 1-3 times per day for subsequent 1-10 weeks.

131. The method of any preceding claim, wherein the method is repeated twice a day for 1-5 weeks and once a day for subsequent 1-10 weeks.

132. The method of any preceding claim, wherein the method is repeated twice a day for 3 weeks and once a day for the remainder of the three months.

133. The method of any preceding claim, wherein the wipe comprises:
a) at least two components adapted to increase a temperature of the wipe from an ambient temperature to a desired temperature upon a reaction of the at least two components,
b) a water-impermeable container for preventing the at least two components from coming into contact with an eye when in use,
c) a seal for keeping the at least two components separate until a temperature change is initiated, and
d) at least one agent selected from the group consisting of soothing agents, cleansing agents, and surfactant agents.

134. The method of claim 133, wherein the method comprises massaging the eye region of the subject with the wipe after the heat is generated.

135. The method of claim 133 or 134, wherein the at least two components comprise an oxidising agent and a reducing agent.

136. The method of any one of claims 133-135, wherein one of the at least two components is a hydratable inorganic salt.

137. The method of claim 136, wherein one of the at least two components is a hydratable sulfate.

138. The method of claim 137, wherein the hydratable sulfate salt is K₂SO₄, Na₂SO₄, Li₂SO₄, MgSO₄, FeSO₄, MnSO₄, CuSO₄, Al₂(SO₄)₃, or ZnSO₄.

139. The method of claim 133 or 134, wherein one of the at least two components is MgSO₄.

140. The method of claim 133 or 134, wherein the at least two components comprise MgSO₄ and water.

141. The method of any one of claims 133-140, wherein the desired temperature is in the range of about 25° C.-56° C.

142. The method of claim 141, wherein the desired temperature is in the range of about 25° C. to about 35° C., about 35° C. to about 40° C., about 40° C. to about 46° C., or about 46° C. to about 53° C.

143. The method of any one of claims 133-142, wherein the desired temperature is reached within 15 seconds to 3 minutes after the temperature change is initiated.

144. The method of claim 143, wherein the desired temperature is reached within about 15 seconds to about 30 seconds, about 30 seconds to about 60 seconds, about 1 minute to about 2 minutes, or about 2 minutes to about 3 minutes after the temperature change is initiated.

145. The method of claim 143, wherein the desired temperature is reached in about: 15 seconds, 30 seconds, 45 seconds, about 1 minute, about 2 minutes, or about 3 minutes after the temperature change is initiated.

146. The method of any one of claims 133-145, wherein the desired temperature is maintained for from about 5 minutes to about 30 minutes.

147. The method of claim 146, wherein the desired temperature is maintained for at least about: 5, 8, 10, 15, 20, 25, or 30 minutes.

148. The method of any one of claims 133-147, wherein the wipe is sterile.

149. The method of any one of claims 133-148, wherein the wipe is square, rectangular, circular, ovoid, eye-shaped, curved to correspond to a contour of an eye, sized to be used on one eye region, or sized to be used on two eyes simultaneously.

150. The method of any one of claims 133-149, wherein the wipe further comprises a band, a pocket, or a layer for holding the wipe for a period of time.

151. The method of claim 150, wherein the band is elastic.

152. The method of any one of claims 133-151, wherein the wipe further comprises an adhesive portion on a part or the whole of one side of the wipe.

153. The method of claim 152, wherein the adhesive portion comprises one or more adhesive materials selected from the group consisting of acrylic adhesives, hydrocolloidal or hydrogel adhesives, natural rubber, synthetic rubber, and any combination thereof.

154. The method of any one of claims 133-153, wherein the wipe further comprises a removal strip to protect an adhesive portion.

155. The method of claim 154, wherein the removal strip is a polymer or paper strip treated with silicone.

156. The method of any one of claims 133-155, wherein the wipe is coated with a polymeric material capable of moulding to a surface of an eye.

157. The method of any one of claims 133-156, wherein the wipe is coated with a polymeric material that softens at a temperature above the ambient temperature.

158. The method of any one of claims 133-157, wherein the seal is a frangible seal.

159. The method of any one of claims 133-158, wherein the at least one agent is at least one cleansing agent or at least one surfactant agent.

160. The method of any one of claims 133-159, wherein the at least one agent is PEG-80 sorbitan laurate, sodium trideceth sulfate, PEG-150 distearate, cocamidopropyl hydroxysultaine, sodium laureth-13 carboxylate, disodium lauroamphodiacetate, polysorbate 80, polysorbate 20, poloxamer 184, ammonium laureth sulfate, ceteareth 20, ceteareth 25, cocamidopropyl betaine, disodium laureth sulfosuccinate, disodium lauriminodipropionate, disodium lauroamphodipropionate, glycol stearate, hydrogenated castor oil, laureth-23, magnesium laureth, oleth sulfate, PEG-20 stearate, PEG-35 castor oil, PEG-40 hydrogenated castor oil, PEG-60 hydrogenated castor oil, PEG-7 hydrogenated castor oil, PEG-75 lanolin, poloxamer, sodium laureth sulfate, sodium trideceth sulfate, sodium C12-15 pareth 15 sulfonate, sodium C14-16 olefin sulfonate, or any combination thereof.

161. The method of any one of claims 133-160, wherein the wipe further comprises a drug delivery system.

162. The method of any one of claims 133-161, wherein the wipe further comprises at least one additional agent selected from the group consisting of anti-inflammatory agents, anti-bacterial agents, decongestants, antistatic agents, preservatives, antioxidants, antimicrobial agents, chelating agents, emollients, emulsifying agents, buffering/neutralising agents, humectants, thickeners, viscosity controlling agents, antistatic agents, conditioning agents, and any combination thereof.

163. The method of claim 162, wherein the wipe comprises a viscosity controlling agent that is polyethylene glycol (PEG).

164. The method of any one of claims 133-163, wherein the at least one additional agent is incorporated in a thermo-responsive polymer layer coated on a surface of the wipe.

165. The method of any one of claims 133-164, wherein the wipe further comprises a reflective layer or a conductive layer to direct heating towards a surface of the wipe.

166. The method of any one of claims 133-165, wherein the wipe further comprises an indicator which confirms to a user that the desired temperature is reached.

167. The method of claim 166, wherein the indicator is a temperature sensitive color indicator configured to change from a first color to a second color when the desired temperature is reached.

168. The method of claim 167, wherein the indicator is configured to undergo a reversible color change, such that when the wipe is no longer at the desired temperature or falls outside of a desired temperature range, the indicator reverts to the first color.

169. The method of any one of claims 166-168, wherein at least a part of the wipe is coated with a temperature reactive ink or a temperature reactive dye.

170. The method of any preceding claim, wherein the subject is a human.
